(19) United States
(12) Patent Application Publication
(10) Pub. No.: US 2018/0250280 A1
(43) Pub. Date: Sep. 6, 2018

(54) SUBSTITUTED OXOPYRIDINE DERIVATIVES

(71) Applicant: BAYER PHARMA AKTIENGESELLSCHAFT, Berlin (DE)

(72) Inventors: Eloisa JIMENEZ NUNEZ, Freiburg im Breisgau (DE); Jens ACKERSTAFF, Düsseldorf (DE); Pascal ELLERBROCK, Düsseldorf (DE); Alexander HILLISCH, Solingen (DE); Katharina MEIER, Mettmann (DE); Stefan HEITMEIER, Wülfrath (DE); Adrian TERSTEEGEN, Wuppertal (DE); Jan STAMPFUß, Düsseldorf (DE)

(73) Assignee: BAYER PHARMA AKTIENGESELLSCHAFT, Berlin (DE)

(21) Appl. No.: 15/757,135

(22) PCT Filed: Aug. 30, 2016
(86) PCT No.: PCT/EP2016/070396

§ 371 Date: Mar. 2, 2018

(30) Foreign Application Priority Data
Sep. 4, 2015 (EP) 15183941.2

(51) Int. Cl. A61K 31/4412; A61K 31/4433; A61K 31/4436; A61K 31/4439; A61K 31/444; A61P 1/00; A61P 7/02; A61P 27/02

(52) U.S. Cl. CPC A61K 31/4412; A61K 31/4433; A61K 31/4436; A61P 27/02; A61K 31/444; A61P 1/00; A61P 7/02; A61K 31/4439

(57) ABSTRACT

The invention relates to substituted oxopyridine derivatives and to processes for preparation thereof, and also to the use thereof for production of medicaments for treatment and/or prophylaxis of diseases, especially of cardiovascular disorders, preferably thrombotic or thromboembolic disorders, and oedemas, and also ophthalmic disorders.

The invention relates to substituted oxopyridine derivatives and to processes for preparation thereof, and also to the use thereof for production of medicaments for treatment and/or prophylaxis of diseases, especially of cardiovascular disorders, preferably thrombotic or thromboembolic disorders, and oedemas, and also ophthalmic disorders.

Blood coagulation is a protective mechanism of the organism which helps to be able to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate in this first phase is low and as a result of the occurrence of TFPI as inhibitor of the TF-FVIIa-FX complex is limited in time.

A central component of the transition from initiation to amplification and propagation of coagulation is factor XIa: in positive feedback loops, thrombin activates, in addition to factor V and factor VIII, also factor XI to factor XIa, whereby factor IX is converted into factor IXa, and, via the factor IXa/factor VIIIa complex generated in this manner, the factor X is activated and thrombin formation is in turn therefore highly stimulated leading to strong thrombus growth and stabilizing the thrombus.

In addition, it has become the focus that, in addition to the stimulation via tissue factor, the coagulation system can be activated particularly on negatively charged surfaces, which include not only surface structures of foreign cells (e.g. bacteria) but also artificial surfaces such as vascular prostheses, stents and extracorporeal circulation. On the surface, initially factor XII (FXII) is activated to factor XIIa which subsequently activates factor XI, attached to cell surfaces, to factor XIa. This leads to further activation of the coagulation cascade as described above. In addition, factor XIIa also activates bound plasma prokallikrein to plasma kallikrein (PK) which, in a potentiation loop, firstly leads to further factor XII activation, overall resulting in amplification of the initiation of the coagulation cascade. In addition, PK is an important bradykinin-releasing protease which, inter alia, thus leads to increased endothelial permeability. Further substrates that have been described are prorenin and prourokinase, whose activation may influence the regulatory processes of the renin-angiotensin system and fibrinolysis. The activation of PK is therefore an important link between coagulative and inflammatory processes.

Uncontrolled activation of the coagulation system or defective inhibition of the activation processes may lead to the formation of local thromboses or embolisms in vessels (arteries, veins, lymph vessels) or cardiac cavities. In addition, systemic hypercoagulability may lead to system-wide formation of thrombi and finally to consumption coagulopathy in the context of a disseminated intravasal coagulation. Thromboembolic complications may also occur in extracorporeal circulatory systems such as during haemodialysis and also in vascular prostheses or prosthetic heart valves and stents.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive activation of coagulation may, by formation of fibrin- and platelet-rich thrombi, lead to thromboembolic disorders and thrombotic complications with life-threatening conditions. Inflammatory processes may also be involved here. Accordingly, thromboembolic disorders are still one of the most frequent causes of morbidity and mortality in most industrialized countries.

The anticoagulants known from the prior art, that is to say substances for inhibiting or preventing blood coagulation, have various disadvantages. Accordingly, in practice, an efficient treatment method or prophylaxis of thrombotic/thromboembolic disorders is found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicamentosa or osteoporosis. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required. In addition, other side-effects such as gastrointestinal problems, hair loss and skin necroses have been described.

More recent approaches for oral anticoagulants are in various phases of clinical evaluation or in clinical use, and have demonstrated their effectiveness in various studies. However, taking these medicaments can also lead to bleeding complications, particularly in predisposed patients. Thus, for antithrombotic medicaments, the therapeutic window is of central importance: The interval between the therapeutically active dose for coagulation inhibition and the dose where bleeding may occur should be as large as possible so that maximum therapeutic activity is achieved at a minimum risk profile.

In various in vitro and in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. In contrast, factor XI deficiency (haemophilia C) did not lead to spontaneous bleeding and was apparent only in the course of surgical operations and trauma, but did show protection with respect to certain thromboembolic events.

In addition, plasma kallikrein (PK) is associated with other disorders, which are associated with increased vascular permeability or chronic inflammatory disorders such as is the case in diabetic retinopathy, macular oedema and hereditary angiooedema or chronic inflammatory intestinal disorders. Diabetic retinopathy is primarily caused by microvascular deficiency, which leads to basal membrane thickening of the vessels and loss of vascularized pericytes followed by vascular occlusion with retinal ischaemia which, owing to the retinal hypoxia thus caused, may lead to enhanced vessel permeability with subsequent formation of a macular oedema and, due to all of the processes present, to the patient going blind. In hereditary angiooedema (HAE), reduced formation of the physiological kallikrein inhibitor C1-esterase inhibitor causes uncontrolled plasma kallikrein activation and hence inflammations with fulminant oedema formation and severe pain. From experimental animal models, there are indications that inhibition of plasma kallikrein inhibits increased vascular permeability and may therefore prevent formation of a macular oedema and/or diabetic retinopathy or may improve the acute symptoms of HAE. Oral plasma kallikrein inhibitors could also be used for prophylaxis of HAE.

The kinins generated by means of plasma kallikrein especially have a causative role in the progression of chronic inflammatory intestinal disorders (CID). Their pro-inflammatory effect via activation of bradykinin receptors induces and potentiates the disease progression. Studies on Crohn's disease patients show a correlation between the kallikrein concentration in the intestinal epithelium and the degree of intestinal inflammation. Activation of the kallikrein-kinin system was likewise observed in experimental animal studies. Inhibition of bradykinin synthesis by kallikrein inhibitors could accordingly be used also for prophylaxis and/or therapy of chronic inflammatory intestinal disorders.

Furthermore, for many disorders the combination of antithrombotic and antiinflammatory principles may also be particularly attractive to prevent the mutual enhancement of coagulation and inflammation.

It is therefore an object of the present invention to provide novel compounds for the treatment of cardiovascular disorders, in particular of thrombotic or thromboembolic disorders, and/or oedematous disorders, and/or ophthalmic disorders, in particular diabetic retinopathy and/or macular oedema, in humans and animals, which compounds have a wide therapeutic bandwidth.

WO 2006/030032 describes inter alia substituted pyridinones as allosteric modulators of the mGluR2 receptor, and WO 2008/079787 describes substituted pyridin-2-ones and their use as glucokinase activators. WO 2014/154794, WO 2014/160592, WO 2015/011087 and WO 2015/063093 describe substituted pyridin-2-ones and their use as factor XIa inhibitors.

The invention provides compounds of the formula

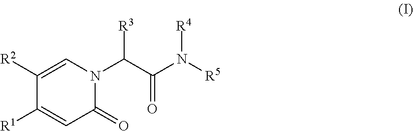

in which
R¹ is a group of the formula

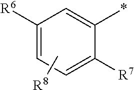

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl, 3,3,3-trifluoro-2-methoxyprop-1-yl or 3,3,3-trifluoro-2-ethoxyprop-1-yl, where alkyl may be substituted by a substituent selected from the group consisting of fluorine, hydroxyl, difluoromethyl, trifluoromethyl, methoxy, ethoxy, tert-butoxy, isopropoxy, difluoromethoxy, trifluoromethoxy, C₃-C₆-cycloalkyl, 4- to 6-membered oxoheterocyclyl, 1,4-dioxanyl, oxazolyl, pyrazolyl, 5,6-dihydro-4H-1,2-oxazin-3-yl, phenyl, pyridyl, C₃-C₆-cycloalkyloxy and 4- to 6-membered oxoheterocyclyloxy,
   in which tert-butoxy and isopropoxy may be substituted by 1 to 3 fluorine substituents,
and
   in which cycloalkyl may be substituted by 1 to 2 substituents selected independently from the group consisting of fluorine, hydroxyl, methyl, ethyl, methoxy, ethoxy, difluoromethyl, trifluoromethyl, difluoromethoxy and trifluoromethoxy,
   and
   in which oxoheterocyclyl may be substituted by 1 to 2 substituents selected independently from the group consisting of fluorine, methyl, ethyl, difluoromethyl and trifluoromethyl,
   and
   in which pyrazolyl is substituted by 1 or 2 substituents selected independently from the group consisting of fluorine, methyl and ethyl,
   and
   in which cycloalkyloxy and oxoheterocyclyloxy may be substituted by 1 to 2 substituents selected independently from the group consisting of fluorine and methyl,
R⁴ is hydrogen,
R⁵ is a group of the formula

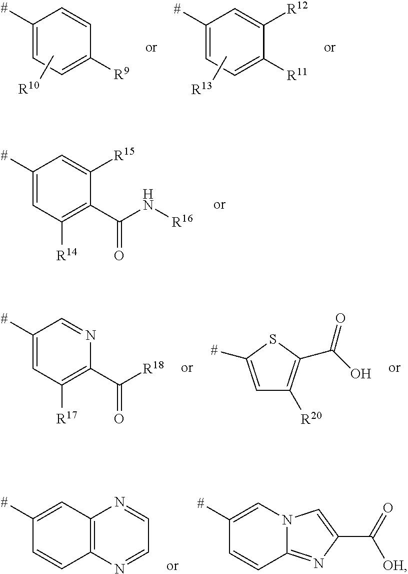

where # is the attachment point to the nitrogen atom,
R⁹ is hydroxycarbonyl or 5-membered heterocyclyl,
R¹⁰ is hydrogen, fluorine or methoxy,
R¹¹ and R¹² together with the carbon atoms to which they are bonded form a 5-membered heterocycle,
   where the heterocycle may be substituted by 1 to 2 substituents selected independently from the group consisting of oxo, hydroxyl, hydroxycarbonyl, methyl, difluoromethyl and trifluoromethyl,
R¹³ is hydrogen or fluorine,
R¹⁴ is hydrogen or fluorine,
R¹⁵ is hydrogen or fluorine,
R¹⁶ is hydrogen, C₁-C₄-alkyl or cyclopropyl,
R¹⁷ is hydrogen or fluorine,
R¹⁸ is hydroxyl or —NHR¹⁹,
   in which
   R¹⁹ is hydrogen, C₁-C₄-alkyl or cyclopropyl,
R²⁰ is hydrogen or fluorine,
and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds according to the invention can occur in tautomeric forms, the present invention encompasses all the tautomeric forms.

In the context of the present invention, the term “enantiomerically pure” is to be understood as meaning that the compound in question with respect to the absolute configuration of the chiral centre is present in an enantiomeric excess of more than 95%, preferably more than 97%. The enantiomeric excess, ee, is calculated here by evaluating the corresponding HPLC chromatogram on a chiral phase using the formula below:

ee=[E^{A}(area %)−E^{B}(area %)]×100%/[E^{A}(area %)+E^{B}(area %)]

(E^{A}: major enantiomer, E^{B}: minor enantiomer)

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to the comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by the processes known to those skilled in the art, for example by the methods described further down and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the compounds according to the invention which form a complex in the solid or liquid state by coordination with solvent molecules. Hydrates are a specific form of the solvates in which the coordination is with water.

The present invention additionally also encompasses prodrugs of the compounds according to the invention. The term “prodrugs” encompasses compounds which for their part may be biologically active or inactive but are converted during their residence time in the body to compounds according to the invention (for example by metabolism or hydrolysis).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl is a straight-chain or branched alkyl radical having 1 to 5 carbon atoms, preferably 1 to 4 carbon atoms, particularly preferably 1 to 3 carbon atoms, by way of example and with preference methyl, ethyl, n-propyl, isopropyl, 2-methylprop-1-yl, n-butyl, tert-butyl and 2,2-dimethylprop-1-yl.

Alkoxy is a straight-chain or branched alkoxy radical having 1 to 4 carbon atoms, preferably 1 to 3 carbon atoms, by way of example and with preference methoxy, ethoxy, n-propoxy, isopropoxy, 2-methylprop-1-oxy, n-butoxy and tert-butoxy.

Cycloalkyl is a monocyclic cycloalkyl group having 3 to 6 carbon atoms; illustrative and preferred examples of cycloalkyl include cyclopropyl, cyclobutyl, cyclopentyl and cyclohexyl.

4- to 6-membered oxoheterocyclyl in the definition of the R³ radical is a saturated monocyclic radical having 4 to 6 ring atoms in which one ring atom is an oxygen atom, by way of example and with preference oxetanyl, tetrahydrofuranyl and tetrahydro-2H-pyranyl.

4- to 6-membered thioheterocyclyl in the definition of the R³ radical is a saturated monocyclic radical having 4 to 6 ring atoms in which one ring atom is a sulfur atom, by way of example and with preference thientanyl, tetrahydrothienyl and tetrahydro-2H-thiopyranyl.

5-Membered heterocyclyl in the definition of the R⁹ radical is a saturated, partly unsaturated or aromatic monocyclic radical having 5 ring atoms and up to 4 heteroatoms from the group of S, O and N, where a nitrogen atom may also form an N-oxide, by way of example and with preference thienyl, furyl, pyrrolyl, thiazolyl, oxazolyl, isoxazolyl, oxadiazolyl, thiadiazolyl, pyrazolyl, imidazolyl, triazolyl, tetrazolyl, dihydrooxazolyl and dihydroimidazolyl.

5-Membered heterocycle in the definition of the R¹¹ and R¹² radicals is a saturated, partly unsaturated or aromatic monocyclic radical having 5 ring atoms and up to 3 heteroatoms, preferably up to 2 heteroatoms, from the group of S, O and N, where a nitrogen atom may also form an N-oxide. This 5-membered heterocycle together with the phenyl ring to which it is bonded, by way of example and with preference, is indolin-5-yl, isoindolin-5-yl, 2,3-dihydro-1H-indazol-5-yl, 2,3-dihydro-1H-benzimidazol-5-yl, 1,3-dihydro-2,1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 2H-indazol-5-yl, 1,2-benzoxazol-5-yl, benzotriazol-5-yl, benzofuran-5-yl, benzothiophen-5-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 2H-indazol-6-yl, 1,2-benzoxazol-6-yl, benzotriazol-6-yl, benzofuran-6-yl and benzothiophen-6-yl.

In the formulae of the group that R¹ may represent, the end point of the line marked by * in each case is not a carbon atom or a CH₂ group, but is part of the bond to the atom to which R¹ is bonded.

In the formulae of the group that R⁵ may represent, the end point of the line marked by # in each case is not a carbon atom or a CH₂ group, but is part of the bond to the atom to which R⁵ is bonded.

Preferred compounds of the formula (I) are those in which
R¹ is a group of the formula

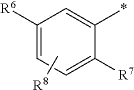

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl, 3,3,3-trifluoro-2-methoxyprop-1-yl or 3,3,3-trifluoro-2-ethoxyprop-1-yl,
   where alkyl may be substituted by a substituent selected from the group consisting of fluorine, hydroxyl, difluoromethyl, trifluoromethyl, methoxy, ethoxy, tert-butoxy, isopropoxy, difluoromethoxy, trifluoromethoxy, C₃-C₆-cycloalkyl, 4- to 6-membered oxoheterocyclyl, 1,4-dioxanyl, oxazolyl, pyrazolyl, 5,6-dihydro-4H-1,2-oxazin-3-yl, phenyl, pyridyl, C₃-C₆-cycloalkyloxy and 4- to 6-membered oxoheterocyclyloxy, in which tert-butoxy and isopropoxy may be substituted by 1 to 3 fluorine substituents,
   and
   in which cycloalkyl may be substituted by 1 to 2 substituents selected independently from the group consisting of fluorine, hydroxyl, methyl, ethyl, methoxy, ethoxy, difluoromethyl, trifluoromethyl, difluoromethoxy and trifluoromethoxy,
   and
   in which oxoheterocyclyl may be substituted by 1 to 2 substituents selected independently from the group consisting of fluorine, methyl, ethyl, difluoromethyl and trifluoromethyl,
   and
   in which pyrazolyl is substituted by 1 or 2 substituents selected independently from the group consisting of fluorine, methyl and ethyl,
   and
   in which cycloalkyloxy and oxoheterocyclyloxy may be substituted by 1 to 2 substituents selected independently from the group consisting of fluorine and methyl,
R⁴ is hydrogen,
R⁵ is a group of the formula

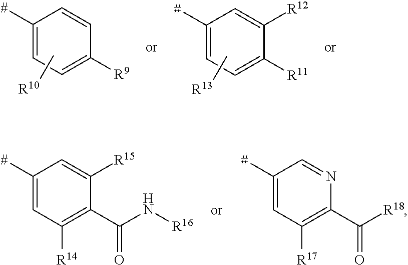

where # is the attachment point to the nitrogen atom,
R⁹ is hydroxycarbonyl or 5-membered heterocyclyl,
R¹⁰ is hydrogen or fluorine,
R¹¹ and R¹² together with the carbon atoms to which they are bonded form a 5-membered heterocycle,
   where the heterocycle may be substituted by 1 to 2 substituents selected independently from the group consisting of oxo, hydroxyl, hydroxycarbonyl, methyl, difluoromethyl and trifluoromethyl,
R¹³ is hydrogen or fluorine,
R¹⁴ is hydrogen or fluorine,
R¹⁵ is hydrogen or fluorine,
R¹⁶ is hydrogen, C₁-C₄-alkyl or cyclopropyl,
R¹⁷ is hydrogen or fluorine,
R¹⁸ is hydroxyl or —NHR¹⁹,
   in which
   R¹⁹ is hydrogen, C₁-C₄-alkyl or cyclopropyl,
and the salts thereof, the solvates thereof and the solvates of the salts thereof.

Preferred compounds of the formula (I) are also those in which
R¹ is a group of the formula

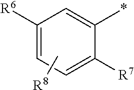

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl,
   where alkyl may be substituted by a substituent selected from the group consisting of methoxy, tert-butoxy, isopropoxy, trifluoromethoxy, 4- to 6-membered oxoheterocyclyl, 1,4-dioxanyl, pyrazolyl, 5,6-dihydro-4H-1,2-oxazin-3-yl and C₃-C₆-cycloalkyloxy,
      in which pyrazolyl is substituted by a methyl substituent,
      and
      in which cycloalkyloxy may be substituted by 1 to 2 methyl substituents,
   R⁴ is hydrogen,
   R⁵ is a group of the formula

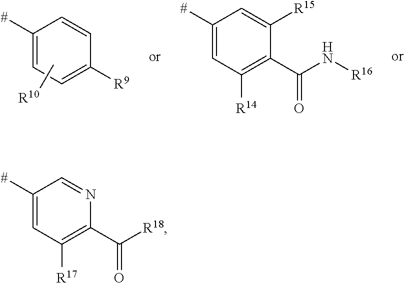

where # is the attachment point to the nitrogen atom,
R⁹ is hydroxycarbonyl,
R¹⁰ is hydrogen or fluorine,
R¹⁴ is hydrogen or fluorine,
R¹⁵ is hydrogen,
R¹⁶ is hydrogen, methyl or ethyl,
R¹⁷ is hydrogen or fluorine,
R¹⁸ is —NHR¹⁹,
   in which
   R¹⁹ is hydrogen, methyl or ethyl,
or
R⁵ is 2H-indazol-5-yl,
   where the 5-membered heterocycle in 2H-indazol-5-yl may be substituted by a substituent selected from the group consisting of methyl, difluoromethyl and trifluoromethyl,
   and
   where the benzyl ring in 2H-indazol-5-yl may be substituted by a fluorine substituent,
and the salts thereof, the solvates thereof and the solvates of the salts thereof.

Preferred compounds of the formula (I) are also those in which
R¹ is a group of the formula

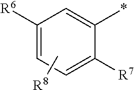

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is ethyl,
   where ethyl is substituted by a substituent selected from the group consisting of methoxy, tetrahydro-2H-pyranyl, 1,4-dioxanyl and pyrazolyl,
      in which pyrazolyl is substituted by a methyl substituent,
   or
   is propyl,
   where propyl is substituted by a methoxy substituent,
R⁴ is hydrogen,
R⁵ is a group of the formula

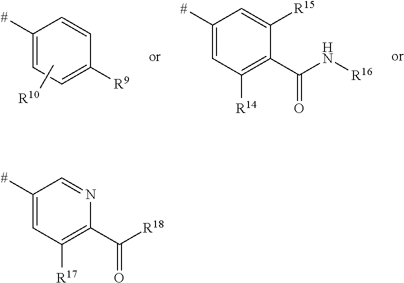

where # is the attachment point to the nitrogen atom,
R⁹ is hydroxycarbonyl,
R¹⁰ is hydrogen,
R¹⁴ is fluorine,
R¹⁵ is hydrogen,
R¹⁶ is hydrogen or methyl,
R¹⁷ is hydrogen,
R¹⁸ is —NHR¹⁹,
   in which
   R¹⁹ is hydrogen or methyl,
or
R⁵ is 2H-indazol-5-yl,
   where the 5-membered heterocycle in 2H-indazol-5-yl is substituted by a methyl substituent,
and the salts thereof, the solvates thereof and the solvates of the salts thereof.

Preferred compounds of the formula (I) are also those in which
R¹ is a group of the formula

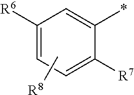

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl,
   where alkyl may be substituted by a substituent selected from the group consisting of methoxy, tert-butoxy, isopropoxy, trifluoromethoxy, 4- to 6-membered oxoheterocyclyl, 1,4-dioxanyl, pyrazolyl, 5,6-dihydro-4H-1,2-oxazin-3-yl and C₃-C₆-cycloalkyloxy,
      in which pyrazolyl is substituted by a methyl substituent,
      and
      in which cycloalkyloxy may be substituted by 1 to 2 methyl substituents,
R⁴ is hydrogen,
R⁵ is a group of the formula

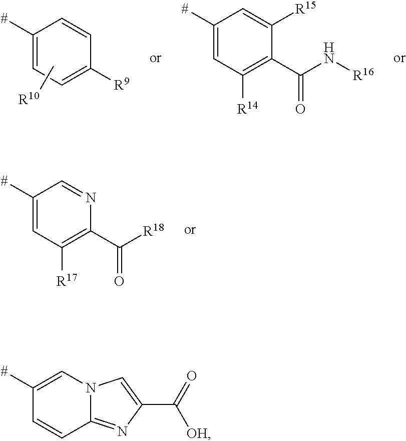

where # is the attachment point to the nitrogen atom,
R⁹ is hydroxycarbonyl,
R¹⁰ is hydrogen or fluorine,
R¹⁴ is hydrogen or fluorine,
R¹⁵ is hydrogen,
R¹⁶ is hydrogen, methyl or ethyl,
R¹⁷ is hydrogen or fluorine,
R¹⁸ is —NHR¹⁹,
   in which
   R¹⁹ is hydrogen, methyl or ethyl,
or
R⁵ is 2H-indazol-5-yl,
   where the 5-membered heterocycle in 2H-indazol-5-yl may be substituted by a substituent selected from the group consisting of methyl, difluoromethyl and trifluoromethyl,
   and
   where the benzyl ring in 2H-indazol-5-yl may be substituted by a fluorine substituent,
and the salts thereof, the solvates thereof and the solvates of the salts thereof.

Preferred compounds of the formula (I) are also those in which
R¹ is a group of the formula

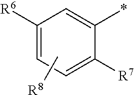

where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is methyl,
   where methyl is substituted by a substituent selected from the group consisting of tetrahydro-2H-pyranyl, 1,4-dioxanyl and pyrazolyl,
      in which pyrazolyl is substituted by a methyl substituent,
   or
   is ethyl,
   where ethyl is substituted by a substituent selected from the group consisting of methoxy,
   or
   is propyl,
   where propyl is substituted by a methoxy substituent,
R⁴ is hydrogen,
R⁵ is a group of the formula

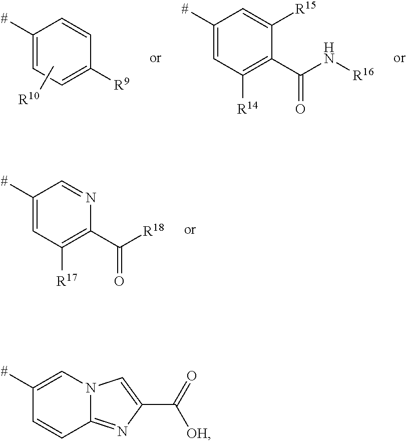

where # is the attachment point to the nitrogen atom,
R⁹ is hydroxycarbonyl,
R¹⁰ is hydrogen,
R¹⁴ is fluorine,
R¹⁵ is hydrogen,
R¹⁶ is hydrogen or methyl,
R¹⁷ is hydrogen,
R¹⁸ is —NHR¹⁹,
   in which
   R¹⁹ is hydrogen or methyl,
or
R⁵ is 2H-indazol-5-yl,
   where the 5-membered heterocycle in 2H-indazol-5-yl is substituted by a methyl substituent,
and the salts thereof, the solvates thereof and the solvates of the salts thereof.

Preferred compounds of the formula (I) are also those in which
R¹ is a group of the formula

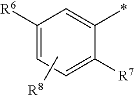

where * is the attachment point to the oxopyridine ring,
R⁶ is chlorine,
R⁷ is 2,2,2-trifluoroethoxy or 2,2-difluoroethoxy,
R⁸ is hydrogen.

Preferred compounds of the formula (I) are also those in which
R⁵ is a group of the formula

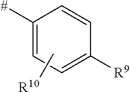

where # is the attachment point to the nitrogen atom,
R⁹ is hydroxycarbonyl,
R¹⁰ is hydrogen.

Preferred compounds of the formula (I) are also those in which
R⁵ is a group of the formula

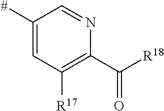

where # is the attachment point to the nitrogen atom,
R¹⁷ is hydrogen,
R¹⁸ is —NHR¹⁹,
   in which
   R¹⁹ is hydrogen or methyl.

Preferred compounds of the formula (I) are also those in which
R⁵ is a group of the formula

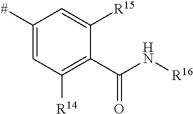

where # is the attachment point to the nitrogen atom,
R¹⁴ is fluorine,
R¹⁵ is hydrogen,
R¹⁶ is hydrogen or methyl.

Preference is also given to compounds of the formula (Ia)

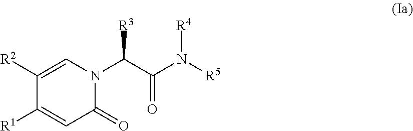

in which R¹, R², R³, R⁴ and R⁵ are as defined above.

The invention further provides a process for preparing the compounds of the formula (I), or the salts thereof, solvates thereof or the solvates of the salts thereof, wherein
[A] the compounds of the formula

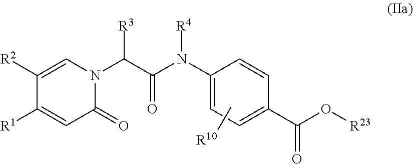

in which
R¹, R², R³, R⁴ and R¹⁰ have the definition given above, and
R²³ is tert-butyl
are reacted with an acid to give compounds of the formula

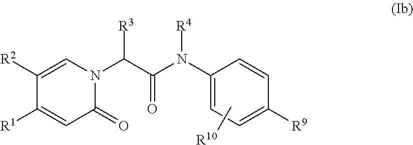

in which
R¹, R², R³, R⁴ and R¹⁰ have the definition given above, and
R⁹ is hydroxycarbonyl,
or
[B] the compounds of the formula

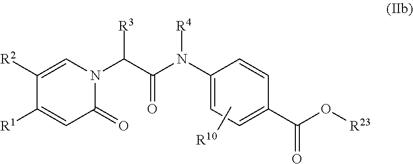

in which
R¹, R², R³, R⁴ and R¹⁰ have the definition given above, and
R²³ is methyl or ethyl,
are reacted with a base to give compounds of the formula

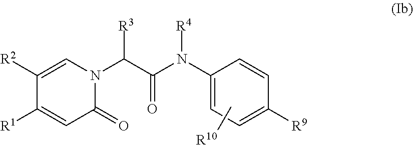

in which
R¹, R², R³, R⁴ and R¹⁰ have the definition given above, and
R⁹ is hydroxycarbonyl,
or
[C] the compounds of the formula

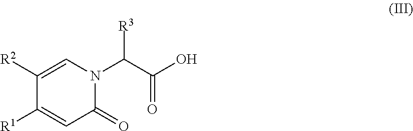

in which
R¹, R² and R³ have the definition given above
are reacted with compounds of the formula

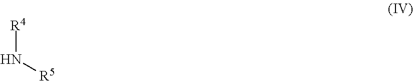

in which
R⁴ and R⁵ have the definition given above,
in the presence of a dehydrating reagent to give compounds of the formula (I),
or
[D] the compounds of the formula

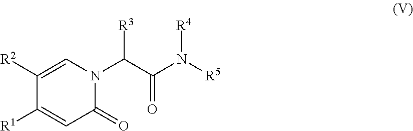

in which
R², R³, R⁴ and R⁵ have the definition given above and
X¹ is chlorine, bromine or iodine
are reacted with compounds of the formula

R¹-Q¹ (VI)

in which
R¹ is as defined above, and
Q¹ is —B(OH)₂, a boronic ester, preferably pinacol boronate, or —BF₃⁻K⁺,
under Suzuki coupling conditions to give compounds of the formula (I).

The compounds of the formula (Ib) are a subset of the compounds of the formula (I).

The compounds of the formulae (IIa) and (IIb) together form the group of the compounds of the formula (II).

The reaction in process [A] is generally effected in inert solvents, preferably within a temperature range from room temperature to 60° C. at standard pressure.

Inert solvents are, for example, halogenated hydrocarbons such as dichloromethane, trichloromethane, carbon tetrachloride or 1,2-dichloroethane, or ethers such as tetrahydrofuran or dioxane, preference being given to dichloromethane.

Acids are, for example, trifluoroacetic acid or hydrogen chloride in dioxane, preference being given to trifluoroacetic acid.

The reaction in process [B] is generally effected in inert solvents, preferably within a temperature range from room temperature up to reflux of the solvents at standard pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water; preference is given to a mixture of tetrahydrofuran and water or a mixture of methanol and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to lithium hydroxide or caesium carbonate.

The reaction in process [C] is generally conducted in inert solvents, if appropriate in the presence of a base, preferably in a temperature range from 0° C. to room temperature at atmospheric pressure. Suitable dehydrating reagents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or 2, 4, 6-tripropyl-1, 3, 5, 2, 4, 6-trioxatriphosphinane 2, 4, 6-trioxide (T3P), or mixtures of these with bases. The condensation is preferably conducted with HATU or T3P.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine. The condensation is preferably conducted with diisopropylethylamine.

Inert solvents are, for example, halogenated hydrocarbons such as dichloromethane or trichloromethane, hydrocarbons such as benzene, or other solvents such as nitromethane, dioxane, dimethylformamide, dimethyl sulfoxide or acetonitrile. It is also possible to use mixtures of the solvents. Particular preference is given to dimethylformamide.

The reaction in process [D] is generally effected in inert solvents, in the presence of a catalyst, optionally in the presence of an additional reagent, optionally in a microwave, preferably in a temperature range from room temperature to 150° C. at atmospheric pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropylbiphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0), [1,1-bis-(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)].

Additional reagents are, for example, potassium acetate, caesium carbonate, potassium carbonate or sodium carbonate, potassium tert-butoxide, caesium fluoride or potassium phosphate, where these may be present in aqueous solution; preferred are additional reagents such as potassium carbonate or aqueous potassium phosphate solution.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulfoxides such as dimethyl sulfoxide, or N-methylpyrrolidone or acetonitrile, or mixtures of the solvents with alcohols such as methanol or ethanol and/or water; preference is given to tetrahydrofuran, dioxane or acetonitrile.

The compounds of the formula (IV) are known, can be synthesized from the corresponding starting compounds by known processes or can be prepared analogously to the processes described in the Examples section.

The compounds of the formula (VI) are known or can be synthesized by known processes from the appropriate starting materials.

The compounds of the formula (II) are known or can be prepared by reacting compounds of the formula

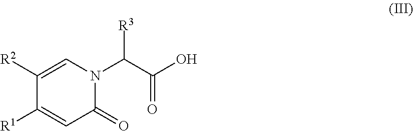

in which
R¹, R² and R³ have the definition given above
with compounds of the formula

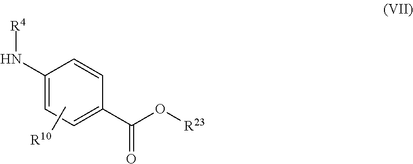

in which
R⁴ and R¹⁰ have the definition given above, and
R²³ is methyl, ethyl or tert-butyl,
in the presence of a dehydrating reagent.

The reaction is effected as described for process [C].

The compounds of the formula (VII) are known, can be synthesized from the corresponding starting compounds by known processes or can be prepared analogously to the processes described in the Examples section.

The compounds of the formula (III) are known or can be prepared by reacting
[E] compounds of the formula

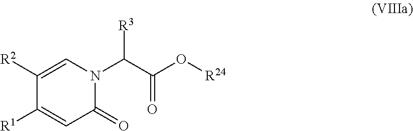

in which
R¹, R² and R³ have the definition given above, and
R²⁴ is tert-butyl,
with an acid,
or
[F] compounds of the formula

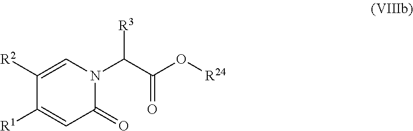

in which
R¹, R² and R³ have the definition given above, and
R²⁴ is methyl, ethyl or benzyl
with a base.

The compounds of the formulae (VIIIa) and (VIIIb) together form the group of the compounds of the formula (VIII).

The reaction in process [E] is effected as described for process [A].

The reaction in process [F] is effected as described for process [B].

The compounds of the formula (VIII) are known or can be prepared by reacting
[G] compounds of the formula

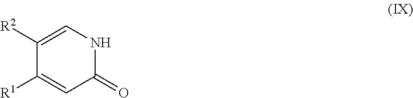

in which
R¹ and R² have the definition given above,
with compounds of the formula

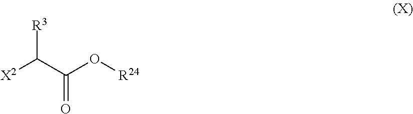

in which
R³ has the definition given above,
R²⁴ is methyl, ethyl, benzyl or tert-butyl, and
X² is chlorine, bromine, iodine, methanesulfonyloxy or trifluoromethanesulfonyloxy,
or
[H] compounds of the formula

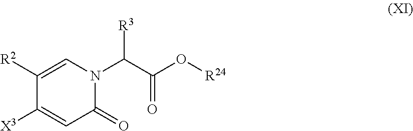

in which
R² and R³ have the definition given above,
R²⁴ is methyl, ethyl, benzyl or tert-butyl, and
X³ is chlorine, bromine or iodine
with compounds of the formula (VI) under Suzuki coupling conditions.

The reaction in process [G] is generally effected in inert solvents, optionally in the presence of a base, preferably in a temperature range from room temperature to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water; preference is given to dimethylformamide.

Bases are, for example, alkali metal hydroxides such as sodium hydroxide, lithium hydroxide or potassium hydroxide, or alkali metal carbonates such as caesium carbonate, sodium carbonate or potassium carbonate, or potassium tert-butoxide or sodium tert-butoxide, sodium hydride or a mixture of these bases or a mixture of sodium hydride and lithium bromide; preference is given to potassium carbonate or sodium hydride.

The compounds of the formula (X) are known or can be synthesized by known processes from the appropriate starting materials.

The reaction in process [H] is effected as described for process [D].

The compounds of the formula (IX) are known or can be prepared by reacting compounds of the formula

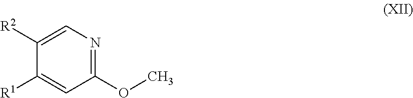

in which
R¹ and R² have the definition given above,
with pyridinium hydrochloride or pyridinium hydrobromide.

The reaction is generally effected in inert solvents, preferably in a temperature range of from 80° C. to 120° C. at atmospheric pressure.

Inert solvents are, for example, hydrocarbons such as benzene, or other solvents such as nitromethane, dioxane, dimethylformamide, dimethyl sulfoxide or acetonitrile. It is also possible to use mixtures of the solvents. Particular preference is given to dimethylformamide.

The compounds of the formula (XII) are known or can be prepared by reacting compounds of the formula

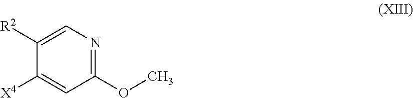

in which
R² is as defined above, and
X⁴ is chlorine, bromine or iodine
with compounds of the formula (VI) under Suzuki coupling conditions.

The reaction is effected as described for process [D].

The compounds of the formula (XIII) are known or can be synthesized by known processes from the appropriate starting compounds.

The compounds of the formula (XI) are known or can be prepared by reacting compounds of the formula

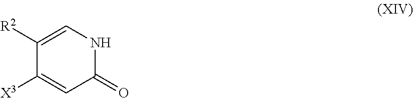

in which
R² is as defined above, and
X³ is chlorine, bromine or iodine
with compounds of the formula (X).

The reaction is effected as described for process [G].

The compounds of the formula (XIV) are known or can be synthesized by known processes from the appropriate starting compounds.

The compounds of the formula (V) are known or can be prepared by reacting compounds of the formula

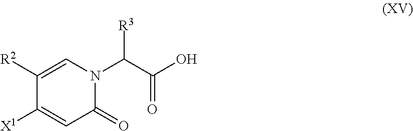

in which
R² and R³ have the definition given above, and
X¹ is chlorine, bromine or iodine
with compounds of the formula (IV) in the presence of a dehydrating reagent.

The reaction is effected as described for process [C].

The compounds of the formula (XV) are known or can be prepared by
[I] reacting compounds of the formula

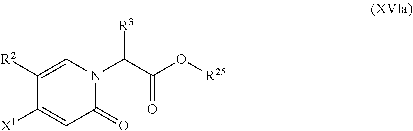

in which
R² and R³ have the definition given above,
R²⁵ is tert-butyl, and
X¹ is chlorine, bromine or iodine
with an acid,
or
[J] reacting compounds of the formula

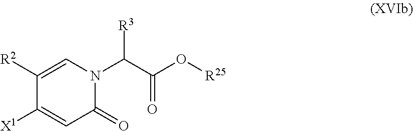

in which
R² and R³ have the definition given above,
R²⁵ is methyl, ethyl or benzyl, and
X¹ is chlorine, bromine or iodine
with a base.

The compounds of the formulae (XVIa) and (XVIb) together form the group of the compounds of the formula (XVI).

The reaction in process [I] is effected as described for process [A].

The reaction in process [J] is effected as described for process [B].

The compounds of the formula (XVI) are known or can be prepared by reacting compounds of the formula

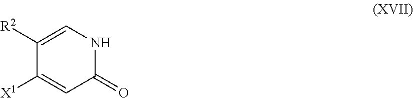

in which
R² has the definition given above, and
X¹ is chlorine, bromine or iodine
with compounds of the formula

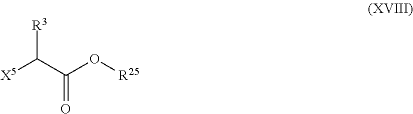

in which
R³ has the definition given above,
R²⁵ is methyl, ethyl, benzyl or tert-butyl, and
X⁵ is chlorine, bromine, iodine, methanesulfonyloxy or trifluoromethanesulfonyloxy.

The reaction is effected as described for process [G].

The compounds of the formulae (XVII) and (XVIII) are known or can be synthesized by known processes from the appropriate starting materials.

In an alternative process, the compounds of the formula (VIII) can be prepared by reacting compounds of the formula

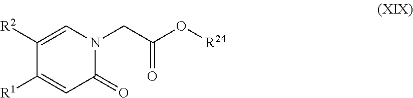

in which
R¹ and R² have the definition given above, and
R²⁴ is methyl, ethyl, benzyl or tert-butyl
with compounds of the formula

R³—X⁶ (XX)

in which
R³ is as defined above, and
X⁶ is chlorine, bromine, iodine, methanesulfonyloxy, trifluoromethanesulfonyloxy or para-toluenesulfonyloxy.

The reaction is generally effected in inert solvents, if appropriate in the presence of a base, preferably in a temperature range from −78° C. to room temperature at atmospheric pressure.

Inert solvents are, for example, halogenated hydrocarbons, such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water; preference is given to tetrahydrofuran.

Bases are, for example, potassium tert-butoxide or sodium tert-butoxide, sodium hydride, N-butyllithium or lithium bis(trimethylsilyl)amide, preference being given to lithium bis(trimethylsilyl)amide.

The compounds of the formula (XIX) are known or can be synthesized by the processes described above, for example process [G], from the appropriate starting materials.

The compounds of the formula (XX) are known or can be synthesized by known processes from the appropriate starting materials.

In an alternative process, the compounds of the formula (VIII) can be prepared by reacting compounds of the formula

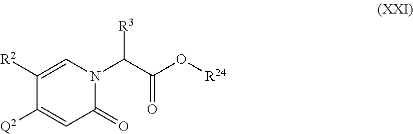

in which
R² and R³ have the definition given above,
R²⁴ is methyl, ethyl, benzyl or tert-butyl, and
Q² is —B(OH)₂, a boronic ester, preferably pinacol boronate, or —BF₃⁻K⁺, with compounds of the formula

R¹—X⁷ (XXII)

in which
R¹ is as defined above, and
X⁷ is chlorine, bromine or iodine,
under Suzuki coupling conditions.

The reaction is effected as described for process [D].

The compounds of the formula (XXI) are known or can be synthesized by known processes from the appropriate starting materials, for example from compounds of the formula (XI).

The compounds of the formula (XXII) are known or can be synthesized by known processes from the appropriate starting materials.

In an alternative process, the compounds of the formula (III) can be prepared by reacting compounds of the formula

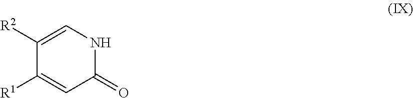

in which
R¹ and R² have the definition given above,
with compounds of the formula

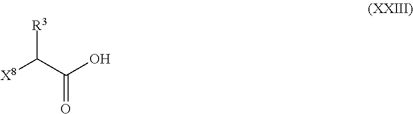

in which
R³ is as defined above, and
X⁸ is chlorine, bromine or iodine.

The reaction is generally effected in inert solvents, if appropriate in the presence of a base, preferably in a temperature range from −10° C. to 90° C. at atmospheric pressure.

Inert solvents are, for example, halogenated hydrocarbons, such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water; preference is given to tetrahydrofuran.

Bases are, for example, potassium tert-butoxide or sodium tert-butoxide, sodium hydride or lithium bis(trimethylsilyl)amide or a mixture of magnesium di-tert-butoxide and potassium tert-butoxide, preference being given to a mixture of magnesium di-tert-butoxide and potassium tert-butoxide.

The compounds of the formula (XXIII) are known or can be synthesized by known processes from the appropriate starting materials.

In an alternative process, the compounds of the formula (XV) can be prepared by reacting compounds of the formula

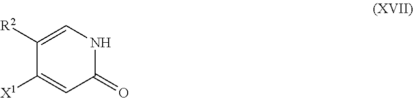

in which
R² has the definition given above, and
X¹ is chlorine, bromine or iodine
with compounds of the formula

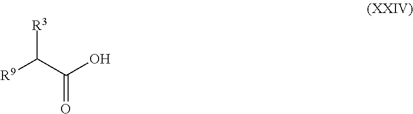

in which
R³ is as defined above, and
X⁹ is chlorine, bromine or iodine.

The reaction is effected as described for the reaction of compounds of the formula (IX) with compounds of the formula (XXIII).

The compounds of the formula (XXIV) are known or can be synthesized by known processes from the appropriate starting materials.

The preparation of the starting compounds and of the compounds of the formula (I) can be illustrated by the synthesis scheme below.

In an alternative process, the compounds of the formula (XIX) can be prepared by reacting compounds of the formula

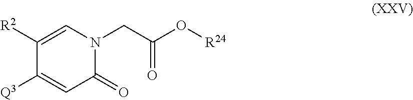

in which
R² has the definition given above,
R²⁴ is methyl, ethyl, benzyl or tert-butyl, and
Q³ is —B(OH)₂, a boronic ester, preferably pinacol boronate, or —BF₃⁻K⁺, with compounds of the formula

R¹—X⁷ (XXII)

in which
R¹ is as defined above, and
X⁷ is chlorine, bromine or iodine.
under Suzuki coupling conditions.

The reaction is effected as described for process [D].

The compounds of the formula (XXV) are known or can be synthesized by known processes from the appropriate starting compounds.

The compounds of the formula (XXII) are known or can be synthesized by known processes from the appropriate starting materials.

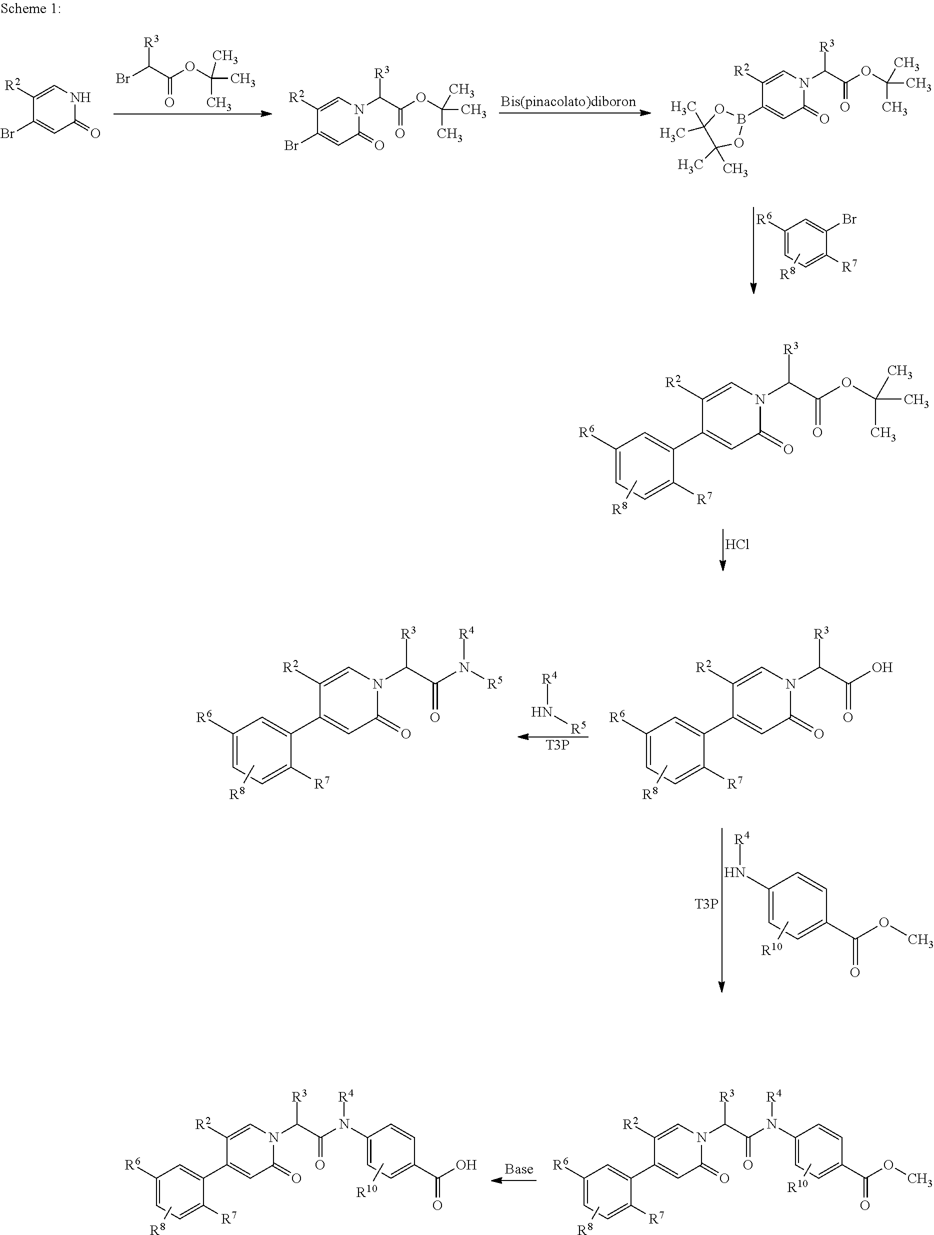

The compounds according to the invention have an unforeseeable useful pharmacological activity spectrum and good pharmacokinetic characteristics. They are compounds that influence the proteolytic activity of the serine protease factor XIa (FXIa) and/or the serine protease plasma kallikrein (PK). The compounds according to the invention inhibit the enzymatic cleavage of substrates, catalysed by FXIa and/or PK, which have essential roles in the activation of blood coagulation, in the aggregation of blood platelets via reduction of the thrombin necessary for the PAR-1 activation of the platelets, and in inflammatory processes, which particularly involve an increase in vascular permeability.

They are therefore suitable for use as medicaments for the treatment and/or prophylaxis of diseases in humans and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications, and/or ophthalmic disorders, in particular of diabetic retinopathy or macular oedema, and/or inflammatory disorders, in particular those associated with excess plasma kallikrein activity, such as hereditary angiooedema (HAE) or chronic inflammatory disorders, particularly of the intestine such as Crohn's disease.

Factor XIa (FXIa) is an important enzyme in the context of coagulation, which can be activated both by thrombin and factor XIIa (FXIIa), and is therefore involved in two essential processes of coagulation: It is a central component of the transition from initiation to amplification and propagation of coagulation: in positive feedback loops, thrombin activates, in addition to factor V and factor VIII, also factor XI to factor XIa, whereby factor IX is converted into factor IXa, and, via the factor IXa/factor VIIIa complex generated in this manner, the factor X is activated and thrombin formation is in turn therefore highly stimulated, leading to strong thrombus growth and stabilizing the thrombus.

Moreover, factor XIa is an important component for the intrinsic initiation of coagulation: In addition to the stimulation via tissue factor (TF), the coagulation system can be activated also particularly on negatively charged surfaces, which include not only surface structures of foreign cells (e.g. bacteria) but also artificial surfaces such as vascular prostheses, stents and extracorporeal circulation. On the surface, initially factor XII (FXII) is activated to factor XIIa (FXIIA) which subsequently activates FXI, attached to cell surfaces, to FXIa. This leads to further activation of the coagulation cascade as described above.

In contrast, thrombin generation in the initiation phase remains uninfluenced via TF/factor VIIa and factor X activation and finally thrombin formation, the physiological reaction on vascular injuries. This could explain why no prolongations of bleeding times were found in FXIa knockout mice, as in rabbits and other species, with administration of FXIa inhibitor. This low bleeding tendency caused by the substance is of great advantage for use in humans, particularly in patients with increased risk of bleeding.

In addition, factor XIIa also activates plasma prokallikrein to plasma kallikrein (PK) in the context of the intrinsic activation which, inter alia, in a potentiation loop, leads to further factor XII activation, overall resulting in amplification of the initiation of the coagulation cascade on surfaces. A PK-inhibiting activity of a compound according to the invention thus reduces coagulation via surface activation and thus has an anticoagulatory effect. An advantage could be in the combination of factor XIa inhibitory activity and PK inhibitory activity allowing a balanced antithrombotic effect.

Accordingly, the compounds according to the invention are suitable for the treatment and/or prophylaxis of disorders or complications which may arise from the formation of clots.

For the purpose of the present invention, the “thrombotic or thromboembolic disorders” include disorders which occur both in the arterial and in the venous vasculature and which can be treated with the compounds according to the invention, in particular disorders in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to peripheral arterial occlusive disorders, pulmonary embolisms, venous thromboembolisms, venous thromboses, in particular in deep leg veins and kidney veins, transitory ischaemic attacks and also thrombotic stroke and thromboembolic stroke.

Stimulation of the coagulation system may occur by various causes or associated disorders. In the context of surgical interventions, immobility, confinement to bed, infections, inflammation or cancer or cancer therapy, inter alia, the coagulation system can be highly activated, and there may be thrombotic complications, in particular venous thromboses. The compounds according to the invention are therefore suitable for the prophylaxis of thromboses in the context of surgical interventions in patients suffering from cancer. The compounds according to the invention are therefore also suitable for the prophylaxis of thromboses in patients having an activated coagulation system, for example in the stimulation situations described.

The inventive compounds are therefore also suitable for the prevention and treatment of cardiogenic thromboembolisms, for example brain ischaemias, stroke and systemic thromboembolisms and ischaemias, in patients with acute, intermittent or persistent cardiac arrhythmias, for example atrial fibrillation, and in patients undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthromboses.

Thromboembolic complications furthermore occur in microangiopathic haemolytical anaemias and by the blood coming into contact with foreign surfaces in the context of extracorporeal circulation, for example haemodialysis, ECMO (“extracorporeal membrane oxygenation”), LVAD (“left ventricular assist device”) and similar methods, AV fistulas, vascular and heart valve prostheses.

Moreover, the compounds according to the invention are suitable for the treatment and/or prophylaxis of disorders involving microclot formation or fibrin deposits in cerebral blood vessels which may lead to dementia disorders such as vascular dementia or Alzheimer's disease. Here, the clot may contribute to the disorder both via occlusions and by binding further disease-relevant factors.

Moreover, the compounds according to the invention are suitable in particular for the treatment and/or prophylaxis of disorders where, in addition to the pro-coagulant component, the pro-inflammatory component also plays an essential role. Mutual enhancement of coagulation and inflammation in particular can be prevented by the compounds according to the invention, thus decisively lowering the probability of thrombotic complications. In this case, both the factor XIa-inhibitory component (via inhibition of thrombin production) and the PK-inhibitory component can contribute to the anticoagulant and antiinflammatory effect (e.g. via bradykinin). Therefore, the treatment and/or prophylaxis in the context of atherosclerotic vascular disorders, inflammations in the context of rheumatic disorders of the locomotor system, inflammatory disorders of the lung, such as pulmonary fibroses, inflammatory disorders of the kidney, such as glomerulonephritides, inflammatory disorders of the intestine, such as Crohn's disease or ulcerative colitis, or disorders which may be present in the context of a diabetic underlying disease, such as diabetic retinopathy or nephropathy, may be considered, inter alia.

Kinins generated by means of plasma kallikrein, inter alia, have a causative role in the progression of chronic inflammatory intestinal disorders (CID). Their pro-inflammatory effect via activation of bradykinin receptors induces and potentiates the disease progression. Studies on Crohn's disease patients show a correlation between the kallikrein concentration in the intestinal epithelium and the degree of intestinal inflammation. Activation of the kallikrein-kinin system was likewise observed in experimental animal studies. Inhibition of bradykinin synthesis by kallikrein inhibitors could accordingly be used also for prophylaxis and/or therapy of chronic inflammatory intestinal disorders.

Moreover, the compounds according to the invention can be used for inhibiting tumour growth and the formation of metastases, and also for the prophylaxis and/or treatment of thromboembolic complications, for example venous thromboembolisms, for tumour patients, in particular those undergoing major surgical interventions or chemo- or radiotherapy.

In addition, the inventive compounds are also suitable for the prophylaxis and/or treatment of pulmonary hypertension.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the substances according to the invention are also useful for the treatment of pulmonary and hepatic fibroses.

In addition, the compounds according to the invention are also suitable for the treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and diffusion of fluid and proteins into the extravasal space. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or crosslinked extravascular tissue. As a consequence, there is coagulation in small vessels of various organs with hypoxia and subsequent organ dysfunction. A secondary effect is the consumption of coagulation factors (for example factor X, prothrombin and fibrinogen) and platelets, which reduces the coagulability of the blood and may result in heavy bleeding.

Compounds according to the invention which inhibit plasma kallikrein alone or in combination with factor XIa, are also useful for the treatment and/or prophylaxis of disorders in the course of which plasma kallikrein is involved. In addition to the anticoagulant activity, plasma kallikrein is an important bradikinin-releasing protease which, inter alia, thus leads to increased endothelial permeability. The compounds can therefore be used for the treatment and/or prophylaxis of disorders involving oedema formations such as ophthalmic disorders, in particular, diabetic retinopathy or macular oedema or hereditary angiooedema.

“Ophthalmic disorders” in the context of the present invention include in particular disorders such as diabetic retinopathy, diabetic macular oedema (DME), macular oedema, macular oedema associated with retinal vein occlusion, age-related macular degeneration (AMD), choroidal neovascularization (CNV), choroidal neovascular membranes (CNVM), cystoid macular oedema (CME), epiretinal membranes (ERM) and macular perforations, myopia-associated choroidal neovascularization, angioid streaks, vascular streaks, retina detachment, atrophic changes of the retinal pigment epithelium, hypertrophic changes of the retinal pigment epithelium, retinal vein occlusion, choroidal retinal vein occlusion, retinitis pigmentosa, Stargardt's disease, retinopathy of prematurity, glaucoma, inflammatory eye disorders such as uveitis, scleritis or endophthalmitis, cataract, refraction anomalies such as myopia, hyperopia or astigmatism and keratoconus, disorders of the anterior eye such as corneal angiogenesis as sequela of, for example, keratitis, cornea transplantation or keratoplasty, corneal angiogenesis as sequela of hypoxia (for example by excessive use of contact lenses), pterygium conjunctivae, subcorneal oedema and intracorneal oedema.

The compounds according to the invention are also suitable for the primary prophylaxis of thrombotic or thromboembolic disorders and/or inflammatory disorders and/or disorders with increased vascular permeability in patients in which gene mutations lead to enhanced activity of the enzymes, or increased levels of the zymogens and these are established by relevant tests/measurements of the enzyme activity or zymogen concentrations.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active ingredients.

In addition, the compounds according to the invention can also be used for preventing coagulation ex vivo, for example for the protection of organs to be transplanted against organ damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may comprise factor XIa or plasma kallikrein.

The present invention furthermore provides a method for preventing the coagulation of blood in vitro, in particular in banked blood or biological samples which may comprise factor XIa or plasma kallikrein or both enzymes, which method is characterized in that an anticoagulatory effective amount of the compound according to the invention is added.

The present invention further provides medicaments comprising a compound according to the invention and one or more further active ingredients, in particular for the treatment and/or prophylaxis of the disorders mentioned above. Preferred examples of suitable combination active ingredients include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or All (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase or plasminogen-modulating substances causing increased formation of plasmin;
anticoagulatory substances (anticoagulants) such as, for example, heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor, elinogrel, PAR-1 antagonists such as, for example, vorapaxar, PAR-4 antagonists, EP3 antagonists such as, for example, DG041;
platelet adhesion inhibitors such as GPVI and/or GPIb antagonists such as, for example, Revacept or caplacizumab;
fibrinogen receptor antagonists (glycoprotein-IIb/IIIa antagonists), for example abciximab, eptifibatide, tirofiban, lamifiban, lefradafiban and fradafiban;
recombinant human activated protein C such as, for example, Xigris or recombinant thrombomudulin;
and also antiarrhythmics;
inhibitors of VEGF and/or PDGF signalling pathways, for example aflibercept, ranibizumab, bevacizumab, KH-902, pegaptanib, ramucirumab, squalamin or bevasiranib, apatinib, axitinib, brivanib, cediranib, dovitinib, lenvatinib, linifanib, motesanib, pazopanib, regorafenib, sorafenib, sunitinib, tivozanib, vandetanib, vatalanib, Vargatef and E-10030;
inhibitors of angiopoietin-Tie signalling pathways such as, for example, AMG386;
inhibitors of Tie2 receptor tyrosine kinase;
inhibitors of the integrin signalling pathways such as, for example, volociximab, cilengitide and ALG1001;
inhibitors of the PI3K-Akt-mTor signalling pathways such as, for example, XL-147, perifosine, MK2206, sirolimus, temsirolimus and everolimus;
corticosteroids such as, for example, anecortave, betamethasone, dexamethasone, triamcinolone, fluocinolone and fluocinolone acetonide;
inhibitors of the ALK1-Smad1/5 signalling pathway such as, for example, ACE041;
cyclooxygenase inhibitors such as, for example, bromfenac and nepafenac;
inhibitors of the kallikrein-kinin system, for example safotibant and ecallantide;
inhibitors of the sphingosine 1-phosphate signalling pathways, for example sonepcizumab;
inhibitors of the complement-C5a receptor, for example eculizumab;
inhibitors of the 5HT1a receptor such as, for example, tandospirone;
inhibitors of the Ras-Raf-Mek-Erk signalling pathway; inhibitors of the MAPK signalling pathways; inhibitors of the FGF signalling pathways; inhibitors of endothelial cell proliferation; apoptosis-inducing active ingredients;
photodynamic therapy consisting of an active ingredient and the action of light, the active ingredient being, for example, verteporfin.

“Combinations” for the purpose of the invention mean not only dosage forms which contain all the components (so-called fixed combinations) and combination packs which contain the components separate from one another, but also components which are administered simultaneously or sequentially, provided that they are used for the prophylaxis and/or treatment of the same disease. It is likewise possible to combine two or more active ingredients with one another, meaning that they are thus each in two-component or multicomponent combinations.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Administration forms suitable for parenteral administration include inter alia preparations for injection and infusion in the form of solutions, suspensions, emulsions, lyophilizates or sterile powders.

Suitable administration forms for extraocular (topic) administration are those which operate in accordance with the prior art, which release the active ingredient rapidly and/or in a modified or controlled manner and which contain the active ingredient in crystalline and/or amorphized and/or dissolved form, for example eye drops, sprays and lotions (e.g. solutions, suspensions, vesicular/colloidal systems, emulsions, aerosols), powders for eye drops, sprays and lotions (e.g. ground active ingredient, mixtures, lyophilizates, precipitated active ingredient), semisolid eye preparations (e.g. hydrogels, in-situ hydrogels, creams and ointments), eye inserts (solid and semisolid preparations, e.g. bioadhesives, films/wafers, tablets, contact lenses).

Intraocular administration includes, for example, intravitreal, subretinal, subscleral, intrachoroidal, subconjunctival, retrobulbar and subtenon administration. Suitable administration forms for intraocular administration are those which operate in accordance with the prior art, which release the active ingredient rapidly and/or in a modified or controlled manner and which contain the active ingredient in crystalline and/or amorphized and/or dissolved form, for example preparations for injection and concentrates for preparations for injection (e.g. solutions, suspensions, vesicular/colloidal systems, emulsions), powders for preparations for injection (e.g. ground active ingredient, mixtures, lyophilizates, precipitated active ingredient), gels for preparations for injection (semisolid preparations, e.g. hydrogels, in-situ hydrogels) and implants (solid preparations, e.g. biodegradable and nonbiodegradable implants, implantable pumps).

Preference is given to oral administration or, in the case of ophthalmologic disorders, extraocular and intraocular administration.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctors.

The present invention further provides medicaments which comprise at least one compound of the invention, preferably together with one or more inert, non-toxic, pharmaceutically suitable excipients, and for the use thereof for the aforementioned purposes.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary, as the case may be, to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for liquid/liquid solutions are based in each case on volume. “w/v” means “weight/volume”. For example, “10% w/v” means: 100 ml of solution or suspension contain 10 g of substance.

### A) EXAMPLES

### Abbreviations

Boc tert-butyloxycarbonyl
Ex. Example
ca. circa
d day(s), doublet (in NMR)
TLC thin-layer chromatography
DCM dichloromethane
DCI direct chemical ionization (in MS)
dd doublet of doublets (in NMR)
DIC N,N′-diisopropylcarbodiimide
DIEA N,N-diisopropylethylamine
DMAP 4-dimethylaminopyridine
DMF N,N-dimethylformamide
DMSO dimethyl sulfoxide
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
HV high vacuum
LC-MS liquid chromatography-coupled mass spectrometry
LDA lithium diisopropylamide
m multiplet (in NMR)
min minute(s)
MS mass spectroscopy
NMR nuclear magnetic resonance spectroscopy
Oxima ethyl hydroxyiminocyanoacetate
q quartet or quadruplet (in NMR)
quant. quantitative
quin quintet (in NMR)
RP reverse phase (in HPLC)
RT room temperature
Rₜ retention time (in HPLC)
s singlet (in NMR)
sxt sextet (in NMR)
SFC supercritical fluid chromatography (with supercritical carbon dioxide as eluent)
t triplet (in NMR)
THF tetrahydrofuran
TFA trifluoroacetic acid
T3P 2,4,6-tripropyl-1,3,5,2,4,6-trioxatriphosphinane 2,4,6-trioxide

### HPLC, LC-MS and GC Methods:

### Method 1:

Instrument: Waters ACQUITY SQD UPLC System; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 208-400 nm.

### Method 2:

Instrument: Waters ACQUITY SQD UPLC System; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 95% A→6.0 min 5% A→7.5 min 5% A; oven: 50° C.; flow rate: 0.35 ml/min; UV detection: 210-400 nm.

### Method 3:

Instrument: Micromass Quattro Premier with Waters UPLC Acquity; column: Thermo Hypersil GOLD 1.9μ 50 mm×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 4:

MS instrument: Waters (Micromass) Quattro Micro; HPLC instrument: Agilent 1100 series; column: YMC-Triart C18 3μ 50 mm×3 mm; eluent A: 1 l water+0.01 mol ammonium carbonate, eluent B: 1 l acetonitrile; gradient: 0.0 min 100% A→2.75 min 5% A→4.5 min 5% A; oven: 40° C.; flow rate: 1.25 ml/min; UV detection: 210 nm.

### Method 5:

MS instrument: Waters (Micromass) QM; HPLC instrument: Agilent 1100 series; column: Agilent ZORBAX Extend-C18 3.0 mm×50 mm 3.5 micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 6:

MS instrument: Waters (Micromass) ZQ; HPLC instrument: Agilent 1100 series; column: Agilent ZORBAX Extend-C18 3.0 mm×50 mm 3.5 micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 7:

Instrument: Thermo DFS, Trace GC Ultra; column: Restek RTX-35, 15 m×200 μm×0.33 μm; constant flow rate of helium: 1.20 ml/min; oven: 60° C.; inlet: 220° C.; gradient: 60° C., 30° C./min→300° C. (hold for 3.33 min).

### Method 8:

Instrument: Agilent MS Quad 6150; HPLC: Agilent 1290; column: Waters Acquity UPLC HSS T3 1.8μ 50 mm×2.1 mm; eluent A: 1 l water+0.25 ml 99% formic acid, eluent B: 1 l acetonitrile+0.25 ml 99% formic acid; gradient: 0.0 min 90% A→0.3 min 90% A→1.7 min 5% A→3.0 min 5% A; oven: 50° C.; flow rate: 1.20 ml/min; UV detection: 205-305 nm.

### Method 9:

Instrument: Thermo Scientific DSQII, Thermo Scientific Trace GC Ultra; column: Restek RTX-35MS, 15 m×200 μm×0.33 μm; constant flow rate of helium: 1.20 ml/min; oven: 60° C.; inlet: 220° C.; gradient: 60° C., 30° C./min→300° C. (hold for 3.33 min).

### Method 10:

MS instrument type Thermo Scientific FT-MS; UHPLC+instrument type Thermo Scientific UltiMate 3000; column Waters, HSST3, 2.1 mm×75 mm, C18μ 1.8 μm; eluent A 1 l of water+0.01% formic acid; eluent B 1 l of acetonitrile+0.01% formic acid; gradient 0.0 min 10% B→2.5 min 95% B→3.5 min 95% B; oven 50° C.; flow rate 0.90 ml/min; UV detection 210 nm/optimum integration path 210-300 nm

### Method 11:

MS instrument: Waters (Micromass) Quattro Micro; instrument: Waters UPLC Acquity; column: Waters BEH C18 1.7μ, 50 mm×2.1 mm; eluent A: 1 l water+0.01 mol ammonium formate, eluent B: 1 l acetonitrile; gradient: 0.0 min 95% A→0.1 min 95% A→2.0 min 15% A→2.5 min 15% A→2.51 min 10% A→3.0 min 10% A; oven: 40° C.; flow rate: 0.5 ml/min; UV detection: 210 nm.

### Microwave:

The microwave reactor used was a “single-mode” instrument of the Emrys™ Optimizer type.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “×HCl”, “×CF₃COOH”, “×Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### Starting Compounds

### General Method 1A: Preparation of a Boronic Acid

To a solution of the appropriate pyridine derivative in tetrahydrofuran (about 3 ml/mmol) at −78° C. was added lithium diisopropylamide (2 M in tetrahydrofuran/heptane/ethylbenzene), the mixture was stirred for 2 to 4 h and then triisopropyl borate was added quickly. The reaction mixture was maintained at −78° C. for a further 2 to 3 h and then slowly thawed to RT overnight. After addition of water, the tetrahydrofuran was removed under reduced pressure and the aqueous phase was extracted twice with ethyl acetate. The aqueous phase was acidified with aqueous hydrochloric acid (2M), generally resulting in formation of a precipitate which was filtered off, washed with water and dried. The aqueous phase was extracted three times with ethyl acetate. The combined organic phases were dried (sodium sulfate or magnesium sulfate), filtered and concentrated under reduced pressure.

### General Method 2A: Suzuki Coupling

A flask which had been dried by heating and flushed with argon was initially charged with 1.0 eq. of the appropriate boronic acids, 1.0 eq. of the aryl bromide or aryl iodide, 3.0 eq. of potassium carbonate and 0.1 eq. of [1,1-bis(diphenylphosphino)ferrocene]palladium(II) chloride/monodichloromethane adduct or tetrakis(triphenylphosphine)palladium(0). The flask was then evacuated three times and in each case vented with argon. Dioxane (about 6 ml/mmol) was added, and the reaction mixture was stirred at 110° C. for a number of hours until substantially complete conversion had been achieved. The reaction mixture was then filtered through Celite and the filtrate was concentrated under reduced pressure. Water was added to the residue. After addition of ethyl acetate and phase separation, the organic phase was washed once with water and once with saturated aqueous sodium chloride solution, dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 3A: Methoxypyridine Cleavage

20 eq. of pyridinium hydrochloride or pyridinium hydrobromide were added to a solution of the appropriate methoxypyridine in dimethylformamide (10-12.5 ml/mmol) and the mixture was stirred at 100° C. for a number of hours to days, with further pyridinium hydrochloride or pyridinium hydrobromide possibly being added, until substantially complete conversion had been achieved. Subsequently, the reaction solution was concentrated under reduced pressure and the residue was stirred with water. The precipitate formed was filtered off, washed with water and dried under reduced pressure.

General Method 4A: N-Alkylation of 2-Pyridinone Derivatives with the Appropriate 2-Bromo- or 2-Chloropropanoic Ester Derivatives in the Presence of Potassium Carbonate

Under argon and at RT, 1.2 eq. of the appropriate 2-bromo- or 2-chloropropanoic ester derivative and 1.5 eq. of potassium carbonate were added to a solution of 1.0 eq. of the appropriate 2-pyridinone derivative in dimethylformamide (5-10 ml/mmol), and the mixture was stirred at 100° C.

After removal of the dimethylformamide and addition of water/ethyl acetate and phase separation, the organic phase was washed with water and with saturated aqueous sodium chloride solution, dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 5A: Amide Coupling Using T3P/Pyridine

A solution of the appropriate carboxylic acid (1 eq.) and the appropriate amine (1.1-1.5 eq.) in pyridine (about 0.1M) was heated to 60 to 80° C., and T3P (50% in ethyl acetate, 1.5 to 4 eq.) was added dropwise. Alternatively, T3P was added at RT and the mixture was then stirred at RT or heated to RT to 90° C. After 1-20 h, the reaction mixture was cooled to RT, and water and ethyl acetate were added. The aqueous phase was extracted with ethyl acetate. The combined organic phases were washed with aqueous buffer solution (pH=5), with saturated aqueous sodium hydrogencarbonate solution and with saturated aqueous sodium chloride solution, dried over sodium sulfate and concentrated under reduced pressure. The crude product was then optionally purified either by normal phase chromatography (eluent: cyclohexane/ethyl acetate mixtures or dichloromethane/methanol mixtures) or by preparative RP-HPLC (water/acetonitrile gradient or water/methanol gradient).

General Method 5B: Amide Coupling with HATU/DIEA

To a solution of the appropriate carboxylic acid (1.0 eq.) in dimethylformamide (7-15 ml/mmol) under argon and at RT were added the amine (1.1 eq.), N,N-diisopropylethylamine (2.2 eq.) and a solution of HATU (1.2 eq.) in a little dimethylformamide. The reaction mixture was stirred at RT. After addition of water/ethyl acetate and phase separation, the organic phase was washed with water and with saturated aqueous sodium chloride solution, dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 6A: Hydrolysis of a Tert-Butyl Ester or a Boc-Protected Amine Using TFA

To a solution of 1.0 eq. of the appropriate tert-butyl ester derivative in dichloromethane (about 5-10 ml/mmol) at RT were added 20 eq. of TFA, and the mixture was stirred at RT for 1 to 8 h. The reaction mixture was then concentrated under reduced pressure and the residue was co-evaporated repeatedly with dichloromethane and toluene and dried under reduced pressure. The crude product was then optionally purified either by normal phase chromatography (cyclohexane/ethyl acetate mixtures or dichloromethane/methanol mixtures) or preparative RP-HPLC (water/acetonitrile gradient or water/methanol gradient).

General Method 6B: Hydrolysis of a Methyl/Ethyl or Benzyl Ester with Lithium Hydroxide

At RT, lithium hydroxide (2-4 eq.) was added to a solution of 1.0 eq. of the appropriate methyl or ethyl ester in tetrahydrofuran/water (3:1, about 7-15 ml/mmol). The reaction mixture was stirred at RT to 60° C. and then adjusted to pH 1 using aqueous hydrochloric acid (1N). After addition of water/ethyl acetate and phase separation, the aqueous phase was extracted three times with ethyl acetate. The combined organic phases were dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 6C: Hydrolysis of a Tert-Butyl Ester Using Lithium Hydroxide

To a solution of 1.0 eq. of the appropriate tert-butyl ester in tetrahydrofuran/ethanol (1:2, 15-50 ml/mmol) at RT was added lithium hydroxide or lithium hydroxide monohydrate (2-5 eq.). The reaction mixture was stirred at RT to 60° C., saturated aqueous ammonium chloride solution was then added and the mixture was adjusted to pH 1 using aqueous hydrochloric acid (1N). After addition of water/ethyl acetate and phase separation, the aqueous phase was extracted three times with ethyl acetate. The combined organic phases were dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 6D: Hydrolysis of a Tert-Butyl Ester Using Hydrogen Chloride in Dioxane

A solution of 1.0 eq. of the appropriate tert-butyl ester derivative in 4M hydrogen chloride in dioxane (concentration of the tert-butyl ester derivative about 0.1M) was either stirred at RT for 2 to 48 h or treated in an ultrasonic bath for 2 to 5 h. The reaction mixture was then concentrated under reduced pressure and the residue was co-evaporated repeatedly with tetrahydrofuran and dried under reduced pressure. The crude product was converted without further purification.

### General Method 7A: Preparation of Triflates

A solution of the appropriate alcohol (1 eq.) was initially charged in dichloromethane (0.1-1M), and at −78° C. to 0° C. lutidine (1.1-1.5 eq.) or triethylamine (1.1-1.5 eq.) or N,N-diisopropylethylamine (1.1-1.5 eq.) and trifluoromethanesulfonic anhydride (1.05-1.5 eq.) were added in succession. The reaction mixture was stirred at −78° C. to 0° C. for another 1 h and then diluted with triple the amount (based on the reaction volume) of methyl tert-butyl ether. The organic phase was washed three times with a 3:1 mixture of saturated aqueous sodium chloride solution/1N hydrochloric acid and finally with saturated aqueous sodium hydrogencarbonate solution, dried (sodium sulfate or magnesium sulfate) and filtered, and the solvent was removed under reduced pressure. The crude product was used in the next stage without further purification.

General Method 8A: Alkylation of Acetic Esters with Triflates

To a solution of the appropriate acetic ester (1 eq.) in tetrahydrofuran (0.1-0.2M) under argon and at −78° C. was added dropwise lithium bis(trimethylsilyl)amide (1.0M in THF, 1.1-1.3 eq.), and the mixture was stirred for 15 min. The appropriate alkyl triflate (1.5-2.0 eq.) was then added neat or as a solution in THF. The resulting reaction mixture was stirred at −78° C. for another 15 min and at RT for another 1 h. Saturated aqueous ammonium chloride solution was added to the reaction mixture. After phase separation, the aqueous phase was extracted with ethyl acetate. The combined organic phases were dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

General Method 9A: Nitro Reduction with Iron

The appropriate nitro compound was dissolved in an ethanol/water mixture (5:1) (about 2-3M), and concentrated hydrochloric acid (0.5-1 eq.) and iron powder (3-8 eq.) were added. The reaction mixture was heated at 80 to 100° C. until the reaction had gone to completion (about 1 to 6 h). The hot reaction mixture was filtered through kieselguhr. The filtercake was washed with methanol and the filtrate was concentrated under reduced pressure. The crude product was then purified either by normal phase chromatography (eluent: cyclohexane/ethyl acetate mixtures or dichloromethane/methanol mixtures) or by preparative RP-HPLC (water/acetonitrile gradient or water/methanol gradient).

### Example 1.1A

### 2-Fluoro-4-nitrobenzamide

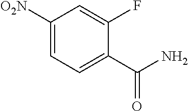 [SMILES: NC(=O)c1ccc([N+](=O)[O-])cc1F]

5.00 g (27 mmol) of 2-fluoro-4-nitrobenzoic acid and 2.17 g (40.5 mmol, 1.5 eq.) of ammonium chloride were reacted according to General Method 5A. The crude product was purified by normal phase chromatography (eluent: dichloromethane/methanol 2-5%). Yield: 2.65 g (53% of theory)

LC/MS [Method 1]: Rₜ=0.48 min; MS (ESIpos): m/z=185 (M+H)⁺,

¹H-NMR (400 MHz, DMSO-d₆): δ [ppm]=8.19 (dd, 1H), 8.12 (dd, 1H), 8.05 (br. s, 1H), 7.91 (br. s, 1H), 7.86 (dd, 1H).

### Example 1.1B

### 4-Amino-2-fluorobenzamide

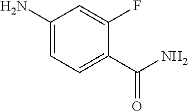 [SMILES: NC(=O)c1ccc(N)cc1F]

2.65 g (14.4 mmol) of 2-fluoro-4-nitrobenzamide were reacted according to General Method 9A.

The crude product was purified by normal phase chromatography (eluent: dichloromethane/methanol 5-10%). Yield: 1.64 g (74% of theory)

LC/MS [Method 5]: Rₜ=0.89 min; MS (ESIpos): m/z=155 (M+H)⁺,

¹H-NMR (400 MHz, DMSO-d₆): δ [ppm]=7.48 (t, 1H), 7.15 (br. s, 1H), 6.97 (br. s, 1H), 6.38 (dd, 1H), 6.27 (dd, 1H), 5.93 (s, 2H).

### Example 1.2A

### 2-Fluoro-N-methyl-4-nitrobenzamide

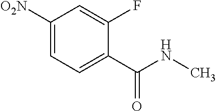

1.00 g (5.40 mmol) of 2-fluoro-4-nitrobenzoic acid and 547 mg (8.10 mmol, 1.5 eq.) of methylamine hydrochloride were reacted according to General Method 5A. Yield: 1.07 g (94% purity, 94% of theory).

LC/MS [Method 1]: Rₜ=0.56 min; MS (ESIpos): m/z=199 (M+H)⁺,

¹H-NMR (400 MHz, DMSO-d₆): δ [ppm]=8.58 (br. s, 1H), 8.20 (dd, 1H), 8.13 (dd, 1H), 7.85 (dd, 1H), 2.80 (d, 3H).

### Example 1.2B

### 4-Amino-2-fluoro-N-methylbenzamide

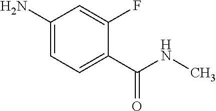

1.07 g (5.07 mmol) of 2-fluoro-N-methyl-4-nitrobenzamide were reacted according to General Method 9A. The crude product was purified by normal phase chromatography (eluent: dichloromethane/methanol 5-10%). Yield: 624 mg (72% of theory)

LC/MS [Method 5]: Rₜ=1.20 min; MS (ESIpos): m/z=169 (M+H)⁺,

¹H-NMR (400 MHz, DMSO-d₆): δ [ppm]=7.54 (br. s, 1H), 7.43 (t, 1H), 6.38 (dd, 1H), 6.27 (dd, 1H), 5.88 (s, 2H), 2.72 (d, 3H).

### Example 1.3A

### 5-Nitropyridine-2-carboxamide

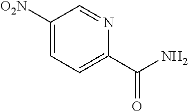 [SMILES: NC(=O)c1ccc([N+](=O)[O-])cn1]

4.00 g (23.8 mmol) of 5-nitropyridine-2-carboxylic acid and 1.91 g (35.7 mmol, 1.5 eq.) of ammonium chloride were reacted according to General Method 5A. After workup, the crude product was used for the next stage without further purification.

LC/MS [Method 1]: Rₜ=0.39 min; MS (ESIpos): m/z=168 (M+H)⁺,

### Example 1.3B

### 5-Aminopyridine-2-carboxamide

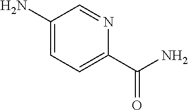

According to General Method 9A, the crude product (about 23.8 mmol) 5-nitropyridine-2-carboxamide was reacted. The product obtained was purified by normal phase chromatography (eluent: dichloromethane/methanol (9:1) with 1.5% concentrated ammonia). Yield: 1.40 g (42% of theory)

LC/MS [Method 5]: Rₜ=0.50 min; MS (ESIpos): m/z=138 (M+H)⁺,

¹H-NMR (400 MHz, DMSO-d₆): δ [ppm]=7.89 (d, 1H), 7.70 (d, 1H), 7.64 (br. s, 1H), 7.11 (br. s, 1H), 6.95 (dd, 1H), 5.90 (s, 2H).

### Example 1.4A

### N-Methyl-5-nitropyridine-2-carboxamide

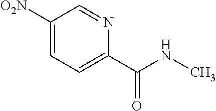

500 mg (2.97 mmol) of 5-nitropyridin-2-carboxylic acid and 301 mg (4.46 mmol, 1.5 eq.) of methylamine hydrochloride were reacted according to General Method 5A. Yield: 459 mg (83% of theory)

LC/MS [Method 3]: Rₜ=1.26 min; MS (ESIpos): m/z=181 (M+H)⁺,

¹H-NMR (400 MHz, DMSO-d₆): δ [ppm]=9.36 (d, 1H), 9.11-8.92 (m, 1H), 8.75 (dd, 1H), 8.26 (d, 1H), 2.85 (d, 3H).

### Example 1.4B

### 5-Amino-N-methylpyridine-2-carboxamide

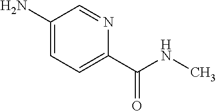

487 mg (2.55 mmol, 1 eq.) of N-methyl-5-nitropyridine-2-carboxamide were reacted according to General Method 9A. The crude product was purified by normal phase chromatography (eluent: dichloromethane/methanol 5-10%). Yield: 225 mg (86% purity, 50% of theory)

¹H-NMR (400 MHz, DMSO-d₆): δ [ppm]=8.32-8.19 (m, 1H), 7.89 (d, 1H), 7.68 (d, 1H), 6.96 (dd, 1H), 5.88 (s, 2H), 2.75 (d, 3H).

### Example 2.1A

### 2-Bromo-4-chlorophenyl-2,2,2-trifluoroethyl ether

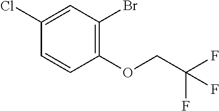 [SMILES: FC(F)(F)COc1ccc(Cl)cc1Br]

To a solution of 6.3 g (29.8 mmol) of 2-bromo-4-chlorophenol in 52.5 ml of DMF were added 26.56 g (148.8 mmol, 5 eq.) of potassium carbonate and 8.0 g (31.2 mmol, 1.05 eq.) of 2,2,2-trifluoroethyl 4-methylbenzenesulfonate, and the mixture was stirred at 110° C. for 18 h. After cooling to RT, 300 ml of water and 300 ml of ethyl acetate were added. After phase separation, the organic phase was washed first with water and then with aqueous 5% lithium chloride solution, dried (sodium sulfate) and concentrated. The crude product was purified by normal phase chromatography (eluent: cyclohexane/ethyl acetate, 0-15%). Yield: 6.8 g (79% of theory)

GC-MS (Method 9): Rₜ=3.75 min; MS (ESIpos): m/z=290 (M+H)⁺,

¹H-NMR (400 MHz, DMSO-d₆): δ [ppm]=7.76 (d, 1H), 7.48 (dd, 1H), 7.27 (d, 1H), 4.88 (q, 2H).

### Example 2.2A

### 2-Bromo-4-chloro-1-(2,2-difluoroethoxy)benzene

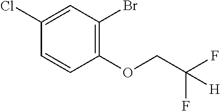 [SMILES: FC(F)COc1ccc(Cl)cc1Br]

800 mg (3.86 mmol) of 2-bromo-4-chlorophenol and 570 μl (4.2 mmol, 1.1 eq.) of 2,2-difluoroethyl trifluoromethanesulfonate were dissolved in 10 ml of DMF, 1.07 g (7.71 mmol, 2.0 eq.) of potassium carbonate were added and the reaction mixture was heated to 90° C. After 16 h, the reaction mixture was cooled down to RT. 10 ml of 1 N hydrochloric acid were added to the reaction mixture, which was extracted with ethyl acetate (three times 10 ml). The combined organic phases were washed with saturated sodium chloride solution, dried over sodium sulfate and concentrated under reduced pressure. The crude product was purified via reverse phase chromatography (75 ml/min, 28 min, 10-95% acetonitrile/water+0.1% formic acid). Yield: 1.02 g (95% of theory).

LC/MS [Method 10]: Rₜ=2.12 min,

¹H-NMR (400 MHz, CDCl₃): δ [ppm]=7.57 (d, 1H), 7.26 (dd, 1H), 6.84 (d, 1H), 6.14 (tt, 1H), 4.21 (td, 2H).

### Example 2.3A

### 2-Bromo-4-chloro-1-(2-fluoroethoxy)benzene

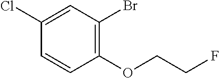 [SMILES: FCCOc1ccc(Cl)cc1Br]

To a solution of 1.94 g (9.16 mmol) of 2-bromo-4-chlorophenol in 16 ml of DMF were added 6.33 g (45.8 mmol, 5 eq.) of potassium carbonate and 2.1 g (9.62 mmol, 1.05 eq.) of 2-fluoroethyl 4-methylbenzenesulfonate, and the mixture was stirred at 110° C. for 18 h. After cooling to RT, 90 ml of water and 90 ml of ethyl acetate were added. After phase separation, the organic phase was washed first with water and then with aqueous 5% lithium chloride solution, dried (sodium sulfate) and concentrated. The crude product was used in the next stage without further purification. Yield: 2.31 g (100% of theory)

GC-MS (Method 9): Rₜ=4.84 min; MS (ESIpos): m/z=254 (M+H)⁺,

¹H-NMR (400 MHz, DMSO-d₆): δ [ppm]=7.70 (d, 1H), 7.42 (dd, 1H), 7.16 (d, 1H), 4.85-4.66 (m, 2H), 4.40-4.26 (m, 2H).

### Example 3.1A

### 2-tert-Butoxyethyl trifluoromethanesulfonate

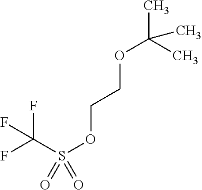

According to General Method 7A, 473 mg (4.00 mmol) of 2-tert-butoxyethanol were reacted at −78° C. with 0.75 ml (4.40 mmol, 1.1 eq.) of trifluoromethanesulfonic anhydride in the presence of 0.61 ml (4.4 mmol, 1.1 eq.) of triethylamine. The crude product was reacted in the next stage without further purification.

¹H-NMR (400 MHz, DMSO-d₆): δ [ppm]=4.38 (t, 2H), 3.57 (t, 2H), 1.19 (s, 9H).

### Example 3.2A

### 2-(Trifluoromethoxy)ethyl trifluoromethanesulfonate

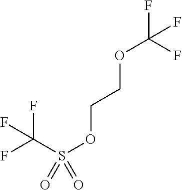 [SMILES: O=S(=O)(OCCOC(F)(F)F)C(F)(F)F]

According to General Method 7A, 200 mg (1.54 mmol) of 2-(trifluoromethoxy)ethanol were reacted at −78° C. with 0.29 ml (1.69 mmol, 1.1 eq.) of trifluoromethanesulfonic anhydride in the presence of 0.24 ml (1.69 mmol, 1.1 eq.) of triethylamine. The crude product was reacted in the next stage without further purification.

¹H-NMR (400 MHz, DMSO-d₆): δ [ppm]=4.59-4.52 (m, 2H), 4.41-4.35 (m, 2H).

### Example 3.3A

### 2-[(Benzyloxy)methyl]tetrahydro-2H-pyran (Racemate)

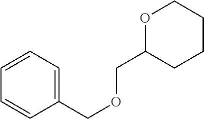 [SMILES: c1ccc(COCC2CCCCO2)cc1]

At 0° C., a solution of 25.0 g (215 mmol) of tetrahydro-2H-pyran-2-ylmethanol (racemate) in 500 ml of THF was slowly added dropwise to a suspension of 9.47 g (237 mmol, 60% in mineral oil) of sodium hydride in 500 ml of THF, and after the addition had ended, the mixture was stirred at 0° C. for another 30 min. 25.7 ml (215 mmol) of benzyl bromide were then added, and the mixture was stirred at 0° C. for another 30 min and at room temperature for another 1 h. The reaction was terminated by addition of 200 ml of saturated aqueous ammonium chloride solution, and the phases were separated. The aqueous phase was extracted twice with 200 ml of methyl tert-butyl ether. The combined organic phases were dried over magnesium sulfate and filtered, and the solvent was removed under reduced pressure. The crude product was purified by column chromatography (ethyl acetate/cyclohexane gradient, 340 g silica cartridge, flow rate: 100 ml/min) and the title compound was obtained. Yield: 41.9 g (94% of theory)

LC/MS [Method 3]: Rₜ=2.18 min; MS (ESIpos): m/z=207 (M+H)⁺,

¹H-NMR (400 MHz, DMSO-d₆): δ [ppm]=7.37-7.25 (m, 5H), 4.47 (s, 2H), 3.87-3.81 (m, 1H), 3.47-3.28 (m, 4H), 1.80-1.72 (m, 1H), 1.58-1.37 (m, 4H), 1.25-1.13 (m, 1H).

### Example 3.3B

### (S)-2-[(Benzyloxy)methyl]tetrahydro-2H-pyran

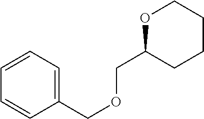 [SMILES: c1ccc(COC[C@@H]2CCCCO2)cc1]

Enantiomer separation of 41.9 g of the racemate from Example 3.3A gave [in addition to 16.7 g of the (R) enantiomer (enantiomer 1): chiral HPLC: Rₜ=5.28 min; 99% ee, purity 93%, optical rotation: [α]₅₈₉^{20.0}=+14.9° (c 0.43 g/100 cm³, chloroform)] 17.0 g of the title compound Example 3.3B (enantiomer 2): chiral HPLC: Rₜ=7.36 min; 96% ee.

Optical rotation: [α]₅₈₉^{20.0}=−13.9° (c 0.61 g/100 cm³, chloroform)

Separating method: Column: OD-H 5 μm 250 mm×20 mm; eluent: 95% isohexane, 5% 2-propanol; temperature: 25° C.; flow rate: 25 ml/min; UV detection: 210 nm.

Analysis: Column: OD-H 5 μm 250 mm×4.6 mm; eluent: 95% isohexane, 5% 2-propanol; flow rate: 1 ml/min; UV detection: 220 nm.

### Example 3.3C

### (2S)-Tetrahydro-2H-pyran-2-ylmethanol

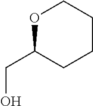

3.51 g (3.30 mmol) of palladium on carbon (10%) were added to a solution of 17.0 g (82.4 mmol) of (S)-2-[(benzyloxy)methyl]tetrahydro-2H-pyran (96% ee) in 120 ml of ethanol, and the mixture was hydrogenated at room temperature and under standard pressure overnight. Another 1.75 g (1.65 mmol) of palladium on carbon (10%) were then added, and hydrogenation was effected at room temperature for a further 72 h. Subsequently, the reaction mixture was filtered through Celite and the filtrate was concentrated. The residue was purified by chromatography (silica, dichloromethane/methanol gradient) and the product fractions were freed of the solvent at <25° C. and >50 mbar. Yield: 8.23 g (86% of theory)

Optical rotation: [α]₅₈₉^{20.0}=+9.1° (c 0.36 g/100 cm³, chloroform), cf. A. Aponick, B. Biannic, Org. Lett. 2011, 13, 1330-1333.

GC/MS [Method 7]: Rₜ=1.82 min; MS: m/z=116 (M)⁺,

¹H-NMR (400 MHz, DMSO-d₆): δ [ppm]=4.51 (t, 1H), 3.87-3.81 (m, 1H), 3.37-3.18 (m, 4H), 1.80-1.71 (m, 1H), 1.59-1.50 (m, 1H), 1.49-1.36 (m, 3H), 1.19-1.05 (m, 1H).

### Example 3.3D

### (2S)-Tetrahydro-2H-pyran-2-ylmethyl trifluoromethanesulfonate

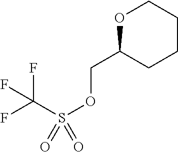 [SMILES: O=S(=O)(OC[C@@H]1CCCCO1)C(F)(F)F]

According to General Method 7A, 330 mg (2.84 mmol) of (2S)-tetrahydro-2H-pyran-2-ylmethanol were reacted with 0.57 ml (3.41 mmol, 1.2 eq.) of trifluoromethanesulfonic anhydride in the presence of 0.48 ml (3.41 mmol, 1.2 eq.) of triethylamine. The crude product was reacted in the next stage without further purification.

¹H-NMR (400 MHz, DMSO-d₆): δ [ppm]=4.32 (dd, 1H), 4.18 (dd, 1H), 4.00-3.92 (m, 1H), 3.60-3.52 (m, 1H), 3.48-3.39 (m, 1H), 1.85-1.74 (m, 1H), 1.56-1.41 (m, 4H), 1.28-1.14 (m, 1H).

### Example 3.4A

### (R)-2-[(Benzyloxy)methyl]tetrahydro-2H-pyran

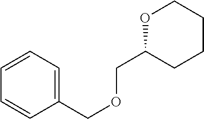 [SMILES: c1ccc(COC[C@H]2CCCCO2)cc1]

Enantiomer separation of 41.9 g of the racemate from Example 3.3A gave 16.7 g of the title compound Example 3.4A (enantiomer 1): chiral HPLC: Rₜ=5.28 min; 99% ee, 93% purity.

Optical rotation: [α]₅₈₉^{20.0}=+14.9° (c 0.43 g/100 cm³, chloroform)

Separating method: Column: OD-H 5 μm 250 mm×20 mm; eluent: 95% isohexane, 5% 2-propanol; temperature: 25° C.; flow rate: 25 ml/min; UV detection: 210 nm.

Analysis: Column: OD-H 5 μm 250 mm×4.6 mm; eluent: 95% isohexane, 5% 2-propanol; flow rate: 1 ml/min; UV detection: 220 nm.

### Example 3.4B

### (2R)-Tetrahydro-2H-pyran-2-ylmethanol

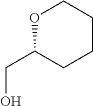

2.06 g (1.94 mmol) of palladium on carbon (10%) were added to a solution of 10.0 g (48.5 mmol) of (R)-2-[(benzyloxy)methyl]tetrahydro-2H-pyran (99% ee) in 70 ml of ethanol, and the mixture was hydrogenated at room temperature and under standard pressure overnight. Another 1.03 g (0.97 mmol) of palladium on carbon (10%) were then added, and hydrogenation was effected at room temperature for a further 72 h. Subsequently, the reaction mixture was filtered through Celite and the filtrate was concentrated. The residue was used in the next stage without further purification. Yield: 5.36 g (95% of theory)

¹H-NMR (400 MHz, DMSO-d₆): δ [ppm]=4.51 (t, 1H), 3.87-3.81 (m, 1H), 3.37-3.18 (m, 4H), 1.80-1.71 (m, 1H), 1.59-1.50 (m, 1H), 1.49-1.36 (m, 3H), 1.19-1.05 (m, 1H).

### Example 3.4C

### (2R)-Tetrahydro-2H-pyran-2-ylmethyl trifluoromethanesulfonate

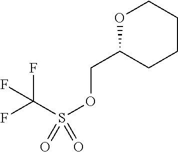 [SMILES: O=S(=O)(OC[C@H]1CCCCO1)C(F)(F)F]

According to General Method 7A, 2.50 g (21.5 mmol) of (2R)-tetrahydro-2H-pyran-2-ylmethanol were reacted with 3.98 ml (23.7 mmol, 1.1 eq.) of trifluoromethanesulfonic anhydride in the presence of 3.3 ml (23.7 mmol, 1.1 eq.) of triethylamine. The crude product was reacted in the next stage without further purification. Yield: 5.4 g (99% of theory).

¹H-NMR (400 MHz, DMSO-d₆): δ [ppm]=4.32 (dd, 1H), 4.18 (dd, 1H), 4.00-3.92 (m, 1H), 3.60-3.52 (m, 1H), 3.48-3.39 (m, 1H), 1.85-1.74 (m, 1H), 1.56-1.41 (m, 4H), 1.28-1.14 (m, 1H).

### Example 3.5A

### Mixture of: 3-(bromomethyl)-5,6-dihydro-4H-1,2-oxazine and 3-(chloromethyl)-5,6-dihydro-4H-1,2-oxazine

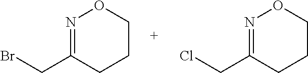 [SMILES: BrCC1=NOCCC1 ; ClCC1=NOCCC1]

Under argon, 330 mg (2.81 mmol) of 5,6-dihydro-4H-1,2-oxazin-3-ylmethanol (synthesized as in V. J. Lee, R. B. Woodward, J. Org. Chem., 1979, 44, 2487-2491) and 0.509 ml (3.65 mmol, 1.3 eq.) of triethylamine were dissolved in 5 ml of DMF and cooled to 0° C. At this temperature, 0.283 ml (3.65 mmol, 1.3 eq.) of methanesulfonyl chloride was added dropwise and the mixture was stirred at 0° C. for 1 h. 683 mg (7.86 mmol, 2.8 eq.) of lithium bromide were then added, and the mixture was stirred at 0° C. for 1 h. 60 ml of water were added and the reaction mixture was extracted three times with ethyl acetate. The combined organic phases were washed with 5% aqueous lithium chloride solution, dried over sodium sulfate and concentrated. The residue was purified by means of Biotage-Isolera (eluent: dichloromethane). Yield: 330 mg (65% of theory) ¹H-NMR (400 MHz, DMSO-d₆): δ [ppm]=4.22 and 4.12 (2×s, 2H), 3.92-3.84 (m, 2H), 2.35-2.22 (m, 2H), 1.89-1.76 (m, 2H).

### Example 3.6A

### 1,4-Dioxan-2-ylmethyl trifluoromethanesulfonate (Racemate)

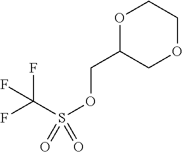 [SMILES: O=S(=O)(OCC1COCCO1)C(F)(F)F]

According to General Method 7A, 1.0 g (8.04 mmol) of 1,4-dioxan-2-ylmethanol were reacted with 1.42 ml (8.44 mmol, 1.05 eq.) of trifluoromethanesulfonic anhydride in the presence of 1.34 ml (9.65 mmol, 1.2 eq.) of triethylamine. The crude product was reacted in the next stage without further purification.

GC/MS [Method 9]: Rₜ=2.91 min; MS: m/z=250 (M)⁺.

### Example 3.7A

### 3-(Bromomethyl)-1-methyl-1H-pyrazole and 3-(chloromethyl)-1-methyl-1H-pyrazole

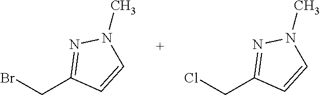

Under argon, 1.0 g (8.47 mmol) of (1-methyl-1H-pyrazol-3-yl)methanol and 1.53 ml (11.0 mmol, 1.3 eq.) of triethylamine were dissolved in 15 ml of DMF and cooled to 0° C. At this temperature, 0.852 ml (11.0 mmol, 1.3 eq.) of methanesulfonyl chloride was added dropwise and the mixture was stirred at 0° C. for 1 h. 2.06 g (23.7 mmol, 2.8 eq.) of lithium bromide were then added, and the mixture was stirred at 0° C. for 1 h. 45 ml of water were added and the reaction mixture was extracted three times with ethyl acetate. The combined organic phases were washed with 3% aqueous lithium chloride solution, dried over sodium sulfate and concentrated. The residue was purified by means of Biotage-Isolera (eluent: dichloromethane). Yield: 1.04 g (67% of theory)

¹H-NMR (400 MHz, DMSO-d₆): δ [ppm]=7.65-7.61 (m, 1H), 6.30-6.26 (m, 1H), 4.66 and 4.58 (2×s, 2H), 3.81 and 3.80 (2×s, 3H).

### Example 3.8A

### 2-Isopropoxyethyl trifluoromethanesulfonate

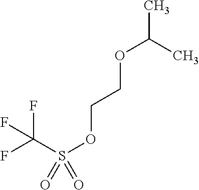

According to General Method 7A, 1.00 g (9.60 mmol) of 2-isopropoxyethanol was reacted with 2.44 ml (14.40 mmol) of trifluoromethanesulfonic anhydride in the presence of 1.68 ml (14.40 mmol) of lutidine. The crude product was reacted in the next stage without further purification.

GC/MS [Method 9]: Rₜ=1.81 min; MS: m/z=177 (M-OCH(CH₃)₂)⁺.

### Example 3.9A

### 2-(Trifluoromethoxy)ethyl trifluoromethanesulfonate

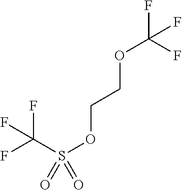 [SMILES: O=S(=O)(OCCOC(F)(F)F)C(F)(F)F]

According to General Method 7A, 500 mg (3.84 mmol) of 3-(trifluoromethoxy)propan-1-ol were reacted with 0.72 ml (4.23 mmol) of trifluoromethanesulfonic anhydride in the presence of 0.59 ml (4.23 mmol) of triethylamine. The crude product was reacted in the next stage without further purification.

¹H-NMR (400 MHz, DMSO-d₆): δ [ppm]=4.59-4.52 (m, 2H), 4.42-4.34 (m, 2H).

### Example 3.10A

### 1-Methylcyclobutanol

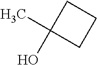

To an initial charge of 5.00 g (71.34 mmol) of cyclobutanone at −10° C. under argon were added dropwise 29.72 ml of methylmagnesium iodide (1.25 M in diethyl ether, 1.25 eq.) within 1 h, and the mixture was stirred at RT for a further 1 h. The reaction mixture was diluted with 20 ml of diethyl ether, 100 ml of hydrochloric acid (1M) were added gradually while cooling with ice, and then the reaction mixture was diluted further with 100 ml of diethyl ether. The organic phase was removed and the aqueous phase was extracted twice with 50 ml each time of diethyl ether. The collected organic phases were washed with saturated aqueous sodium chloride solution, dried over sodium sulfate, filtered and concentrated at 20° C. under slightly reduced pressure (150 mbar). The crude product obtained in this manner was reacted without further purification. Yield: 5.87 g (90% purity, 86% of theory).

¹H-NMR (400 MHz, DMSO-d₆): δ [ppm]=3.32 (br. s., 1H), 2.03-1.91 (m, 2H), 1.87-1.77 (m, 2H), 1.61-1.34 (m, 2H), 1.21 (s, 3H).

### Example 3.10B

### 2-[(1-Methylcyclobutyl)oxy] ethanol

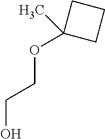

An initial charge of 6.43 g (95%, 0.254 mol) of sodium hydride in 30.0 ml of THF under argon was brought to 0° C. Subsequently, a solution of 6.11 ml (0.085 mol) of bromoacetic acid in 30.0 ml of THF was added dropwise and the reaction mixture was stirred at 0° C. for a further 30 min. Thereafter, a solution of 4.87 g (0.057 mol) of 1-methylcyclobutanol in 30.0 ml of THF was added dropwise at 0° C. and the mixture was stirred at 0° C. for 30 min and at RT overnight. The reaction mixture was diluted with 75 ml of diethyl ether and then acidified at 0° C. with 4M hydrochloric acid. The organic phase was removed and the aqueous phase was extracted with 50 ml of diethyl ether. The collected organic phases were washed with saturated aqueous sodium chloride solution, dried over magnesium sulfate, filtered and concentrated at 30° C. under slightly reduced pressure (100 mbar). The crude product thus obtained (14.3 g, 50% purity), without further purification, was taken up in 145.0 ml of THF and then, at 0° C., 115.2 ml of lithium aluminium hydride (2.4M in THF, 0.277 mol) were added dropwise. The mixture was then brought to RT and stirred for another 30 min. Subsequently, the reaction mixture was cooled to 0° C., and 50 ml of water were added in portions. The suspension obtained was diluted with 100 ml of THF, 50 ml of potassium hydroxide solution (15% in water) were added, and the mixture was filtered through Celite. The filter residue was washed with 200 ml of THF and the collected filtrate was washed with saturated aqueous sodium chloride solution, dried over magnesium sulfate, filtered and concentrated at 20° C. under slightly reduced pressure (80 mbar). The residue was separated by means of normal phase chromatography (cyclohexane/ethyl acetate gradient) and the product fractions were concentrated at 20° C. and 80 mbar. Yield: 3.29 g.

¹H-NMR (400 MHz, DMSO-d₆): δ [ppm]=3.44 (q, 2H), 3.31 (s, 1H), 3.28-3.20 (m, 2H), 2.09-1.99 (m, 2H), 1.81-1.71 (m, 2H), 1.69-1.47 (m, 2H), 1.26 (s, 3H).

### Example 3.10C

### 2-[(1-Methylcyclobutyl)oxy]ethyl trifluoromethanesulfonate

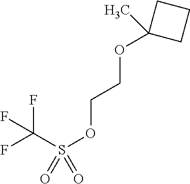 [SMILES: CC1(OCCOS(=O)(=O)C(F)(F)F)CCC1]

To an initial charge of 911 mg (7.00 mmol) of 2-[(1-methylcyclobutyl)oxy]ethanol in 10 ml of dichloromethane were added 1.07 ml (7.7 mmol) of triethylamine at −70° C., and finally 1.24 ml (7.35 mmol) of trifluoromethanesulfonic anhydride were added dropwise. The mixture was stirred at −70° C. for a further 30 min, and then 30 ml of a mixture of saturated aqueous sodium chloride solution and 1 N hydrochloric acid (3:1) were added. Thereafter, the mixture was extracted with 50 ml of diethyl ether, the phases were separated and the aqueous phase was re-extracted with diethyl ether. The collected organic phases were washed three times with 50 ml each time of a mixture of saturated aqueous sodium chloride solution and 1 N hydrochloric acid (3:1) and once with saturated aqueous sodium chloride solution, dried over magnesium sulfate, filtered and concentrated at 20° C. under slightly reduced pressure (100 mbar). The crude product thus obtained was reacted further without further purification.

### Example 3.11A

### (2S)-2-Methoxypropyl trifluoromethanesulfonate

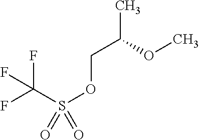

According to General Method 7A, 700 mg (7.77 mmol) of (S)-(+)-2-methoxypropanol were reacted with 1.38 ml (8.16 mmol, 1.05 eq.) of trifluoromethanesulfonic anhydride in the presence of 1.19 ml (8.54 mmol, 1.10 eq.) of triethylamine. The crude product was reacted in the next stage without further purification.

### Example 4.1A

### tert-Butyl (4-bromo-5-methoxy-2-oxopyridin-1(2H)-yl)acetate

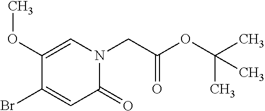 [SMILES: COc1cn(CC(=O)OC(C)(C)C)c(=O)cc1Br]

12.0 g (58.8 mmol) of 4-bromo-5-methoxypyridin-2(1H)-one and 12.2 g (88.2 mmol, 1.5 eq.) of potassium carbonate were initially charged in 267 ml of DMF, 10.6 ml (70.6 mmol, 1.2 eq.) of tert-butyl bromoacetate were added and the mixture was stirred at 50° C. for 80 min. The reaction mixture was then concentrated. 120 ml of water were added, the mixture was stirred for 5 min and filtered off with suction and the product was washed with water, suspended in acetonitrile and concentrated. The crude product was purified by normal phase chromatography (eluent: dichloromethane/methanol, 0-12%). Yield: 15.0 g (80% of theory).

LC/MS [Method 10]: Rₜ=1.49 min; MS (ESIpos): m/z=318 (M+H)⁺,

¹H-NMR (400 MHz, DMSO-d₆): δ [ppm]=7.53 (s, 1H), 6.85 (s, 1H), 4.53 (s, 2H), 3.69 (s, 3H), 1.42 (s, 9H).

### Example 4.1B

### tert-Butyl [5-methoxy-2-oxo-4-(4,4,5,5-tetramethyl-1,3,2-dioxaborolan-2-yl)pyridin-1(2H)-yl]acetate

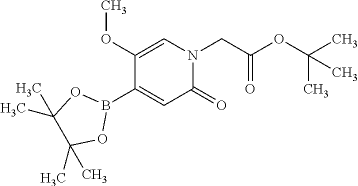 [SMILES: COc1cn(CC(=O)OC(C)(C)C)c(=O)cc1B1OC(C)(C)C(C)(C)O1]

To an initial charge of 4.00 g (12.6 mmol) of tert-butyl (4-bromo-5-methoxy-2-oxopyridin-1(2H)-yl)acetate (racemate), 3.51 g (13.8 mmol, 1.1 eq.) of bis(pinacolato)diboron and 3.7 g (37.7 mmol, 3 eq.) of potassium acetate in 120 ml of dioxane under argon were added 308 mg (0.377 mmol, 0.03 eq) of [1,1-bis(diphenylphosphino)ferrocene]dichloropalladium dichloromethane complex, and the mixture was stirred at 80° C. for 16 h. The reaction mixture was cooled and filtered through kieselguhr, and the filter cake was washed with dichloromethane and acetonitrile. The filtrate was concentrated and dried at 40° C. under high vacuum. Yield: 7.39 g (62% purity, quant.). 1H-NMR (400 MHz, DMSO-d₆): δ [ppm]=7.24 (s, 1H), 6.49 (s, 1H), 4.49 (s, 2H), 3.57 (s, 3H), 1.41 (s, 9H), 1.26 (s, 12H).

### Example 4.2A

### tert-Butyl 2-(4-bromo-5-methoxy-2-oxopyridin-1(2H)-yl)-4-methoxybutanoate (Racemate)

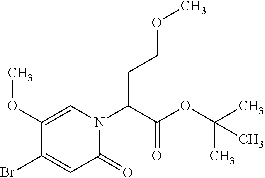 [SMILES: COCCC(C(=O)OC(C)(C)C)n1cc(OC)c(Br)cc1=O]

Under argon and at −70° C., 15 ml (1.0M in THF, 1.35 eq.) of bis(trimethylsilyl)lithium amide were added dropwise to a solution of 3.6 g (10.9 mmol) of tert-butyl (4-bromo-5-methoxy-2-oxopyridin-1(2H)-yl)acetate in 138 ml of tetrahydrofuran, and the mixture was stirred for 20 min. Subsequently, 1.93 ml (12.5 mmol, 1.15 eq.) of 2-methoxyethyl trifluoromethanesulfonate were added dropwise, and the mixture was stirred at −70° C. for 15 min and at RT for 1.5 h. The reaction mixture was cooled to −70° C. again, 4.9 ml (1.0M in THF, 0.45 eq.) of bis(trimethylsilyl)lithium amide were added dropwise followed, after 15 min, by 0.65 ml (4.2 mmol, 0.39 eq.) of 2-methoxyethyl trifluoromethanesulfonate, and the mixture was stirred at −70° C. for 15 min and at RT for 3 h. First 40 ml of saturated aqueous ammonium chloride solution and then 40 ml of water and 350 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-60%). Yield 3.09 g (95% purity, 72% of theory)

LC-MS [Method 1]: Rₜ=0.94 min; MS (ESIpos): m/z=376 (M+H)⁺,

¹H-NMR (400 MHz, DMSO-d₆): δ [ppm]=7.36 (s, 1H), 6.85 (s, 1H), 5.04 (dd, 1H), 3.71 (s, 3H), 3.39-3.29 (m, 1H), 3.20-3.03 (m, 4H), 2.35-2.20 (m, 2H), 1.38 (s, 9H).

### Example 4.2B

### tert-Butyl 4-methoxy-2-[5-methoxy-2-oxo-4-(4,4,5,5-tetramethyl-1,3,2-dioxaborolan-2-yl)pyridin-1(2H)-yl]butanoate (Racemate)

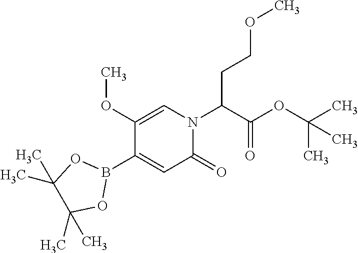 [SMILES: COCCC(C(=O)OC(C)(C)C)n1cc(OC)c(B2OC(C)(C)C(C)(C)O2)cc1=O]

6.00 g (15.5 mmol) of tert-butyl 2-(4-bromo-5-methoxy-2-oxopyridin-1(2H)-yl)-4-methoxybutanoate (racemate), 4.32 g (17.0 mmol, 1.1 eq.) of bis(pinacolato)diboron and 4.55 g (46.4 mmol, 3 eq.) of potassium acetate were initially charged in 84 ml of dioxane under argon, 379 mg (0.464 mmol, 0.03 eq) of [1,1-bis(diphenylphosphino)ferrocene]dichloropalladium dichloromethane complex were added and the mixture was stirred at 80° C. for 6 h. The reaction mixture was cooled and filtered through kieselguhr, and the filter cake was washed with dioxane. The filtrate was concentrated and dried at 40° C. under high vacuum. Yield: 9.90 g (66% purity, quant.).

¹H-NMR (400 MHz, DMSO-d₆): δ [ppm]=7.09 (s, 1H), 6.49 (s, 1H), 5.00 (dd, 1H), 3.60 (s, 3H), 3.36-3.27 (m, 3H), 3.17 (s, 3H), 3.14-3.05 (m, 1H), 2.30-2.21 (m, 2H), 1.37 (s, 9H), 1.27 (s, 12H).

### Example 4.3A

### tert-Butyl 2-(4-bromo-5-methoxy-2-oxopyridin-1 (2H)-yl)-4-tert-butoxybutanoate (Racemate)

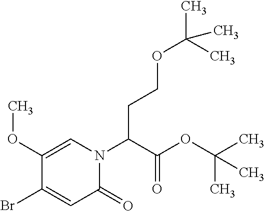 [SMILES: COc1cn(C(CCOC(C)(C)C)C(=O)OC(C)(C)C)c(=O)cc1Br]

To a solution of 5.4 g (16.9 mmol) of tert-butyl (4-bromo-5-methoxy-2-oxopyridin-1(2H)-yl)acetate in 250 ml of tetrahydrofuran under argon and at −70° C. were added dropwise 22.9 ml (1.0M in THF, 1.35 eq.) of bis(trimethylsilyl)lithium amide, and the mixture was stirred for 20 min. Subsequently, 5.3 g (92% purity, 19.5 mmol, 1.15 eq.) of 2-tert-butoxyethyl trifluoromethanesulfonate were added dropwise, and the mixture was stirred at −70° C. for 15 min and at RT for 1.5 h. First 100 ml of saturated aqueous ammonium chloride solution and then 100 ml of water and 300 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-50%). Yield: 4.73 g (65% of theory)

LC/MS [Method 1]: Rₜ=1.14 min; MS (ESIpos): m/z=418 (M+H)⁺,

¹H-NMR (400 MHz, DMSO-d₆): δ [ppm]=7.36 (s, 1H), 6.83 (s, 1H), 5.08 (dd, 1H), 3.72 (s, 3H), 3.37-3.22 (m, 1H), 3.15-3.06 (m, 1H), 2.37-2.15 (m, 2H), 1.38 (s, 9H), 1.04 (s, 9H).

### Example 4.3B

### tert-Butyl 4-tert-butoxy-2-[5-methoxy-2-oxo-4-(4,4,5,5-tetramethyl-1,3,2-dioxaborolan-2-yl)pyridin-1(2H)-yl]butanoate (Racemate)

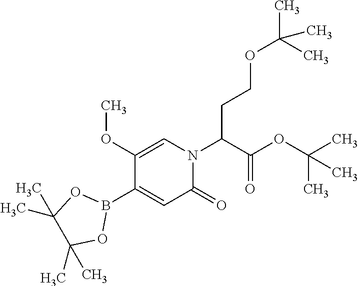 [SMILES: COc1cn(C(CCOC(C)(C)C)C(=O)OC(C)(C)C)c(=O)cc1B1OC(C)(C)C(C)(C)O1]

To an initial charge of 4.7 g (11.3 mmol) of tert-butyl 2-(4-bromo-5-methoxy-2-oxopyridin-1(2H)-yl)-4-tert-butoxybutanoate (racemate), 3.15 g (12.4 mmol, 1.1 eq.) of bis(pinacolato)diboron and 3.32 g (33.9 mmol, 3 eq.) of potassium acetate in 110 ml of dioxane under argon were added 277 mg (0.339 mmol, 0.03 eq) of [1,1-bis(diphenylphosphino)ferrocene]dichloropalladium dichloromethane complex, and the mixture was stirred at 80° C. for 16 h. The reaction mixture was cooled and filtered through kieselguhr, and the filter cake was washed with dichloromethane and acetonitrile. The filtrate was concentrated and dried at 40° C. under high vacuum. Yield: 7.68 g (68% purity, quant.).

¹H-NMR (400 MHz, DMSO-d₆): δ [ppm]=7.08 (s, 1H), 6.48 (s, 1H), 5.03 (dd, 1H), 3.60 (s, 3H), 3.35-3.25 (m, 1H), 3.12-3.04 (m, 1H), 2.31-2.13 (m, 2H), 1.37 (s, 9H), 1.26 (s, 12H), 1.05 (s, 9H).

### Example 4.4A

### tert-Butyl 2-(4-bromo-5-methoxy-2-oxopyridin-1(2H)-yl)-3-(1,4-dioxan-2-yl)propanoate (Diastereomer Mixture)

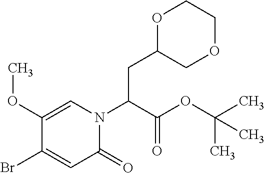 [SMILES: COc1cn(C(CC2COCCO2)C(=O)OC(C)(C)C)c(=O)cc1Br]

To a solution of 1.64 g (4.95 mmol) of tert-butyl (4-bromo-5-methoxy-2-oxopyridin-1(2H)-yl)acetate in 63 ml of tetrahydrofuran under argon and at −70° C. were added dropwise 6.7 ml (1.0M in THF, 1.35 eq.) of bis(trimethylsilyl)lithium amide, and the mixture was stirred for 20 min. Subsequently, 1.5 g (5.7 mmol, 1.15 eq.) of 1,4-dioxan-2-ylmethyl trifluoromethanesulfonate were added dropwise, and the mixture was stirred at −70° C. for 15 min and at RT for 1.5 h. First 30 ml of saturated aqueous ammonium chloride solution and then 30 ml of water and 150 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-65%). Yield: 1.59 g (73% of theory)

LC/MS [Method 10]: Rₜ=1.64 min; MS (ESIpos): m/z=420 (M+H)⁺.

### Example 4.4B

### tert-Butyl 3-(1,4-dioxan-2-yl)-2-[5-methoxy-2-oxo-4-(4,4,5,5-tetramethyl-1,3,2-dioxaborolan-2-yl)pyridin-1(2H)-yl]propanoate (Diastereomer Mixture)

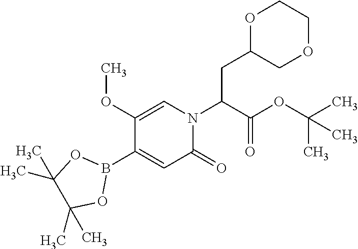 [SMILES: COc1cn(C(CC2COCCO2)C(=O)OC(C)(C)C)c(=O)cc1B1OC(C)(C)C(C)(C)O1]

To an initial charge of 560 mg (1.3 mmol) of tert-butyl 2-(4-bromo-5-methoxy-2-oxopyridin-1(2H)-yl)-3-(1,4-dioxan-2-yl)propanoate (diastereomer mixture), 366 mg (1.44 mmol, 1.1 eq.) of bis(pinacolato)diboron and 386 mg (3.9 mmol, 3 eq.) of potassium acetate in 13.6 ml of dioxane under argon were added 32 mg (39 μmol, 0.03 eq) of [1,1-bis(diphenylphosphino)ferrocene]dichloropalladium dichloromethane complex, and the mixture was stirred at 80° C. for 4.5 h. The reaction mixture was cooled and filtered through kieselguhr, and the filter cake was washed with dioxane. The filtrate was concentrated and dried at 40° C. under high vacuum. The crude product was used in the next stage without further purification. Yield: 1.13 g (53% purity, 98% of theory).

### Example 4.5A

### tert-Butyl 2-(4-bromo-5-methoxy-2-oxopyridin-1(2H)-yl)-3-(5,6-dihydro-4H-1,2-oxazin-3-yl)propanoate (Racemate)

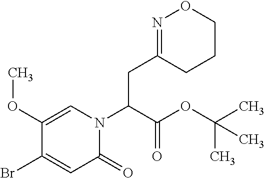 [SMILES: COc1cn(C(CC2=NOCCC2)C(=O)OC(C)(C)C)c(=O)cc1Br]

To a solution of 642 mg (1.94 mmol) of tert-butyl (4-bromo-5-methoxy-2-oxopyridin-1(2H)-yl)acetate in 24.7 ml of tetrahydrofuran under argon and at −70° C. were added dropwise 2.62 ml (1.0M in THF, 1.35 eq.) of bis(trimethylsilyl)lithium amide, and the mixture was stirred for 20 min. Subsequently, 329 mg (2.2 mmol, 1.15 eq.) of a mixture of 3-(bromomethyl)-5,6-dihydro-4H-1,2-oxazine and 3-(chloromethyl)-5,6-dihydro-4H-1,2-oxazine were added and the mixture was stirred at −70° C. for 15 min and at RT for 2 h. First 20 ml of saturated aqueous ammonium chloride solution and then 20 ml of water and 100 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-55%). Yield 200 mg (80% purity, 20% of theory)

LC/MS [Method 10]: Rₜ=1.62 min; MS (ESIpos): m/z=417 (M+H)⁺,

### Example 4.5B

### tert-Butyl 3-(5,6-dihydro-4H-1,2-oxazin-3-yl)-2-[5-methoxy-2-oxo-4-(4,4,5,5-tetramethyl-1,3,2-dioxaborolan-2-yl)pyridin-1(2H)-yl]propanoate (Racemate)

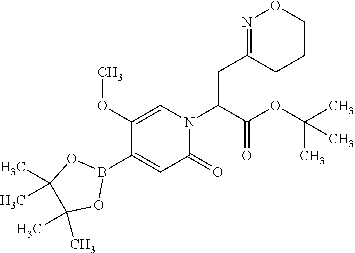 [SMILES: COc1cn(C(CC2=NOCCC2)C(=O)OC(C)(C)C)c(=O)cc1B1OC(C)(C)C(C)(C)O1]

To an initial charge of 200 mg (80% purity, 0.385 mmol) of tert-butyl 2-(4-bromo-5-methoxy-2-oxopyridin-1(2H)-yl)-3-(5,6-dihydro-4H-1,2-oxazin-3-yl)propanoate (racemate), 107 mg (0.42 mmol, 1.1 eq.) of bis(pinacolato)diboron and 113 mg (1.16 mmol, 3 eq.) of potassium acetate in 4 ml of dioxane under argon were added 9.4 mg (12 μmol, 0.03 eq) of [1,1-bis(diphenylphosphino)ferrocene]dichloropalladium dichloromethane complex, and the mixture was stirred at 80° C. for 5 h. The reaction mixture was cooled and filtered through kieselguhr, and the filter cake was washed with dioxane. The filtrate was concentrated and dried at 40° C. under high vacuum. The raw material was used in the next stage without further purification. Yield: 360 mg (about 49% purity, 99% of theory).

### Example 4.6A

### tert-Butyl 2-(4-bromo-5-methoxy-2-oxopyridin-1 (2H)-yl)-4-isopropoxybutanoate (Racemate)

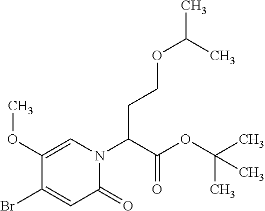 [SMILES: COc1cn(C(CCOC(C)C)C(=O)OC(C)(C)C)c(=O)cc1Br]

To a solution of 1.00 g (3.14 mmol) of tert-butyl (4-bromo-5-methoxy-2-oxopyridin-1(2H)-yl)acetate in 25.0 ml of THF under argon and at −78° C. were added dropwise 4.09 ml of bis(trimethylsilyl)lithium amide (1.0M in THF, 4.09 mmol), and the mixture was stirred for 15 min. Subsequently, 0.89 g (3.77 mmol) of 2-isopropoxyethyl trifluoromethanesulfonate in 10 ml of THF were added dropwise and the mixture was stirred at −78° C. for a further 15 min. The reaction mixture was brought to RT while stirring and stirred for a further 1 h. Subsequently, 50 ml of saturated aqueous ammonium chloride solution were added and the mixture was extracted three times with 50 ml each time of ethyl acetate. The combined organic phases were washed with 30 ml of saturated aqueous sodium chloride solution, dried over magnesium sulfate, filtered and concentrated under reduced pressure. The residue was purified by means of normal phase chromatography (cyclohexane/ethyl acetate gradient). Yield: 813 mg (63% of theory)

LC/MS [Method 1]: Rₜ=1.98 min; MS (ESIpos): m/z=404 (M+H)⁺,

¹H-NMR (400 MHz, DMSO-d₆): δ [ppm]=7.35 (s, 1H), 6.84 (s, 1H), 5.10-5.00 (m, 1H), 3.72 (s, 3H), 3.45-3.34 (m, 2H), 3.17-3.07 (m, 1H), 2.36-2.16 (m, 2H), 1.38 (s, 9H), 1.07-0.96 (m, 6H).

### Example 4.6B

### tert-Butyl 4-isopropoxy-2-[5-methoxy-2-oxo-4-(4,4,5,5-tetramethyl-1,3,2-dioxaborolan-2-yl)pyridin-1(2H)-yl]butanoate (Racemate)

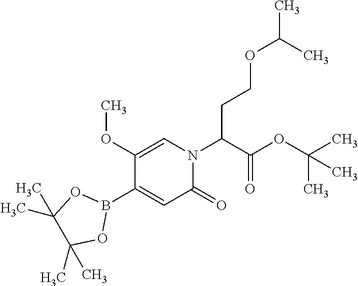 [SMILES: COc1cn(C(CCOC(C)C)C(=O)OC(C)(C)C)c(=O)cc1B1OC(C)(C)C(C)(C)O1]

To an initial charge of 350 mg (0.87 mmol) of tert-butyl 2-(4-bromo-5-methoxy-2-oxopyridin-1(2H)-yl)-4-isopropoxybutanoate (racemate), 242 mg (0.95 mmol) of bis(pinacolato)diboron and 255 mg (2.60 mmol) of potassium acetate in 10.0 ml of dioxane under argon were added 21 mg (0.03 mmol) of [1,1-bis(diphenylphosphino)ferrocene]dichloropalladium dichloromethane complex, and the mixture was stirred at 80° C. for 3 h. The reaction mixture was cooled, filtered through kieselguhr and washed through three times with dioxane. The filtrate was concentrated and dried under high vacuum. Yield: 606 mg (64% purity, quant.).

¹H-NMR (400 MHz, DMSO-d₆): δ [ppm]=7.10 (s, 1H), 6.49 (s, 1H), 5.05-4.98 (m, 1H), 3.60 (s, 3H), 3.44-3.33 (m, 2H), 3.13-3.03 (m, 1H), 2.31-2.17 (m, 2H), 1.39-1.34 (m, 9H), 1.16 (s, 12H), 1.05-0.98 (m, 6H).

### Example 4.7A

### tert-Butyl 2-(4-bromo-5-methoxy-2-oxopyridin-1(2H)-yl)-4-(trifluoromethoxy)butanoate (Racemate)

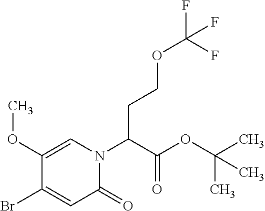 [SMILES: COc1cn(C(CCOC(F)(F)F)C(=O)OC(C)(C)C)c(=O)cc1Br]

To a solution of 850 mg (2.67 mmol) of tert-butyl (4-bromo-5-methoxy-2-oxopyridin-1(2H)-yl)acetate in 27.0 ml of THF under argon and at −78° C. were added dropwise 3.48 ml of bis(trimethylsilyl)lithium amide (1.0M in THF, 3.48 mmol), and the mixture was stirred for 15 min. Subsequently, 841 mg of 3-(trifluoromethoxy)propyl trifluoromethanesulfonate (3.21 mmol) in 10 ml of THF were added dropwise and the mixture was stirred at −78° C. for a further 15 min. The reaction mixture was brought to RT while stirring and stirred for a further 2 h. Subsequently, 80 ml of saturated aqueous ammonium chloride solution were added and the mixture was extracted three times with 100 ml each time of ethyl acetate. The combined organic phases were washed with 100 ml of saturated aqueous sodium chloride solution, dried over magnesium sulfate, filtered and concentrated under reduced pressure. The residue was purified by means of normal phase chromatography (cyclohexane/ethyl acetate gradient). Yield: 658 mg (56% of theory)

LC/MS [Method 1]: Rₜ=2.01 min; MS (ESIpos): m/z=430 (M+H)⁺,

¹H-NMR (400 MHz, DMSO-d₆): δ [ppm]=7.45-7.39 (m, 1H), 6.87 (s, 1H), 5.09-5.01 (m, 1H), 4.20-4.09 (m, 1H), 4.04-3.94 (m, 1H), 3.70 (s, 3H), 2.55-2.40 (m, 2H, partly concealed), 1.38 (s, 9H).

### Example 4.7B

### tert-Butyl 2-[5-methoxy-2-oxo-4-(4,4,5,5-tetramethyl-1,3,2-dioxaborolan-2-yl)pyridin-1(2H)-yl]-4-(trifluoromethoxy)butanoate (Racemate)

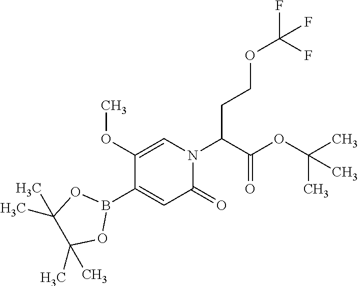 [SMILES: COc1cn(C(CCOC(F)(F)F)C(=O)OC(C)(C)C)c(=O)cc1B1OC(C)(C)C(C)(C)O1]

To an initial charge of 350 mg (0.81 mmol) of tert-butyl 2-(4-bromo-5-methoxy-2-oxopyridin-1(2H)-yl)-4-(trifluoromethoxy)butanoate (racemate), 227 mg (0.90 mmol) of bis(pinacolato)diboron and 240 mg (2.44 mmol) of potassium acetate in 10.0 ml of dioxane under argon were added 20 mg (0.02 mmol) of [1,1-bis(diphenylphosphino)ferrocene]dichloropalladium dichloromethane complex, and the mixture was stirred at 80° C. for 3 h. The reaction mixture was cooled, filtered through kieselguhr and washed through three times with dioxane. The filtrate was concentrated and dried under high vacuum. Yield: 578 mg (67% purity, quant.).

¹H-NMR (400 MHz, DMSO-d₆): δ [ppm]=7.17 (s, 1H), 6.50 (s, 1H), 5.05-4.97 (m, 1H), 4.14-4.08 (m, 1H), 3.97-3.89 (m, 1H), 3.57 (s, 3H), 2.47-2.42 (m, 2H, partly concealed), 1.37 (s, 9H), 1.28 (s, 12H).

### Example 4.8A

### tert-Butyl (4S)-2-(4-bromo-5-methoxy-2-oxo-1-pyridyl)-4-methoxypentanoate (Racemate)

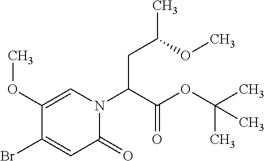 [SMILES: COc1cn(C(C[C@H](C)OC)C(=O)OC(C)(C)C)c(=O)cc1Br]

To a solution of 2.00 g (6.27 mmol) of tert-butyl (4-bromo-5-methoxy-2-oxopyridin-1(2H)-yl)acetate in 40.0 ml of THF under argon and at −70° C. were added dropwise 8.49 ml of bis(trimethylsilyl)lithium amide (1.0M in THF, 8.49 mmol), and the mixture was stirred for 30 min. Subsequently, 1.79 g of (2S)-2-methoxypropyl trifluoromethanesulfonate (90% purity, 7.23 mmol) were added dropwise and the mixture was stirred at −70° C. for a further 30 min. The reaction mixture was brought to RT while stirring and stirred for a further 30 min. Subsequently, 50 ml of saturated aqueous ammonium chloride solution were added and the mixture was extracted twice with 50 ml each time of ethyl acetate. The combined organic phases were washed with 30 ml of saturated aqueous sodium chloride solution, dried over magnesium sulfate, filtered and concentrated under reduced pressure. The residue was purified by means of normal phase chromatography (cyclohexane/ethyl acetate gradient). Yield 1.45 g (80% purity, 47% of theory)

LC/MS [Method 1]: Rₜ=0.95 min; MS (ESIpos): m/z=390 (M+H)⁺,

¹H-NMR (400 MHz, DMSO-d₆): δ [ppm]=7.44-7.33 (m, 1H), 6.88-6.81 (m, 1H), 5.18-5.06 (m, 1H), 3.72 (s, 3H), 3.16-3.09 (m, 3.5H), 2.96-2.85 (m, 0.5H), 2.35-2.25 (m, 1H), 2.25-1.96 (m, 2H), 1.41-1.34 (m, 9H), 1.10-1.02 (m, 3H).

### Example 4.8B

### tert-Butyl (4S)-4-methoxy-2-[5-methoxy-2-oxo-4-(4,4,5,5-tetramethyl-1,3-dioxolan-2-yl)-1-pyridyl]pentanoate (Racemate)

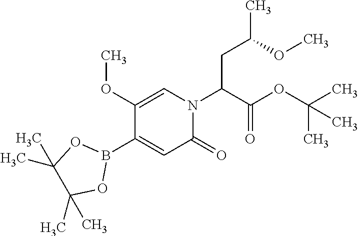 [SMILES: COc1cn(C(C[C@H](C)OC)C(=O)OC(C)(C)C)c(=O)cc1B1OC(C)(C)C(C)(C)O1]

To an initial charge of 1.45 g (3.72 mmol) of tert-butyl (4S)-2-(4-bromo-5-methoxy-2-oxo-1-pyridyl)-4-methoxypentanoate (racemate), 1.04 g (4.09 mmol) of bis(pinacolato)diboron and 1.09 g (11.15 mmol) of potassium acetate in 45.0 ml of dioxane under argon was added 0.09 g (0.03 mmol) of [1,1-bis(diphenylphosphino)ferrocene]dichloropalladium dichloromethane complex, and the mixture was stirred at 80° C. for 3 h. The reaction mixture was cooled down, filtered through kieselguhr and washed through with 100 ml of dioxane. The filtrate was concentrated and dried under high vacuum. Yield: 2.98 mg (40% purity, 90% of theory).

LC/MS [Method 10]: Rₜ=1.26 min; MS (ESIpos): m/z=356 (M+H)⁺.

### Example 4.9A

### tert-Butyl 2-(4-bromo-5-methoxy-2-oxopyridin-1(2H)-yl)-3-[(2S)-tetrahydro-2H-pyran-2-yl]propanoate (Diastereomer Mixture)

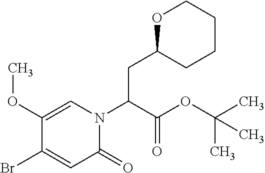 [SMILES: COc1cn(C(C[C@@H]2CCCCO2)C(=O)OC(C)(C)C)c(=O)cc1Br]

To a solution of 1.75 g (5.50 mmol) of tert-butyl (4-bromo-5-methoxy-2-oxopyridin-1(2H)-yl)acetate in 80 ml of tetrahydrofuran under argon and at −70° C. were added dropwise 7.4 ml (1.0M in THF, 1.35 eq.) of bis(trimethylsilyl)lithium amide, and the mixture was stirred for 20 min. Subsequently, 1.62 g (6.33 mmol, 1.15 eq.) of (2S)-tetrahydro-2H-pyran-2-ylmethyl trifluoromethanesulfonate were added dropwise, and the mixture was stirred at −70° C. for 15 min and at RT for 1.5 h. First 30 ml of saturated aqueous ammonium chloride solution and then 30 ml of water and 100 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 20-35%). Yield 1.77 g (94% purity, 72% of theory)

LC/MS [Method 1]: Rₜ=1.04 min; MS (ESIpos): m/z=416 (M+H)⁺.

### Example 4.9B

### tert-Butyl 2-[5-methoxy-2-oxo-4-(4,4,5,5-tetramethyl-1,3,2-dioxaborolan-2-yl)pyridin-1(2H)-yl]-3-[(2S)-tetrahydro-2H-pyran-2-yl]propanoate (Diastereomer Mixture)

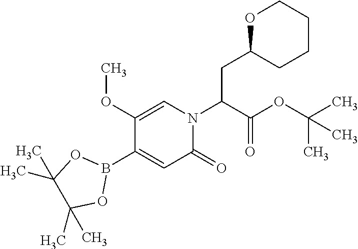 [SMILES: COc1cn(C(C[C@@H]2CCCCO2)C(=O)OC(C)(C)C)c(=O)cc1B1OC(C)(C)C(C)(C)O1]

To an initial charge of 1.77 g (3.98 mmol, 94% purity) of tert-butyl 2-(4-bromo-5-methoxy-2-oxopyridin-1(2H)-yl)-3-[(2S)-tetrahydro-2H-pyran-2-yl]propanoate (diastereomer mixture), 1.11 g (4.37 mmol, 1.1 eq.) of bis(pinacolato)diboron and 1.17 g (11.9 mmol, 3 eq.) of potassium acetate in 40 ml of dioxane under argon were added 97.4 mg (119 μmol, 0.03 eq) of [1,1-bis(diphenylphosphino)ferrocene]dichloropalladium dichloromethane complex, and the mixture was stirred at 80° C. for 18 h. The reaction mixture was cooled and filtered through kieselguhr, and the filter cake was washed with dioxane. The filtrate was concentrated and dried at 40° C. under high vacuum. The crude product was used in the next stage without further purification. Yield: 2.74 g (67% purity, 100% of theory).

### Example 4.10A

### tert-Butyl 2-(4-bromo-5-methoxy-2-oxopyridin-1(2H)-yl)-4-[(1-methylcyclobutyl)oxy]butanoate (Racemate)

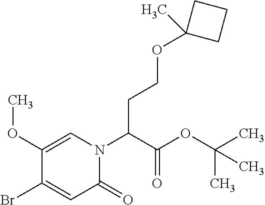 [SMILES: COc1cn(C(CCOC2(C)CCC2)C(=O)OC(C)(C)C)c(=O)cc1Br]

To a solution of 1.80 g (5.66 mmol) of tert-butyl (4-bromo-5-methoxy-2-oxopyridin-1(2H)-yl)acetate in 36 ml of tetrahydrofuran under argon were added dropwise, at −70° C., 7.64 ml (1.0M in THF, 1.35 eq.) of bis(trimethylsilyl)lithium amide, and the mixture was stirred for a further 30 min. Subsequently, 1.71 g (6.51 mmol, 1.15 eq.) of 2-[(1-methylcyclobutyl)oxy]ethyl trifluoromethanesulfonate were added dropwise, and the mixture was stirred at −70° C. for 30 min and at RT for 1.5 h and left to stand overnight. 50 ml of saturated aqueous ammonium chloride solution were added, and the reaction mixture was extracted twice with 50 ml each time of ethyl acetate. The combined organic phases were washed with 30 ml of saturated aqueous sodium chloride solution, dried (magnesium sulfate), filtered and concentrated. The crude product was purified by normal phase chromatography (eluent: cyclohexane/ethyl acetate gradient). Yield: 1.35 g (55% of theory)

LC/MS [Method 1]: Rₜ=1.12 min; MS (ESIpos): m/z=430 (M+H)⁺.

¹H-NMR (400 MHz, DMSO-d₆): δ [ppm]=7.38 (s, 1H), 6.84 (s, 1H), 5.13-5.06 (m, 1H), 3.72 (s, 3H), 3.32-3.25 (m, partly concealed), 3.09-3.01 (m, 1H), 2.37-2.19 (m, 2H), 2.04-1.82 (m, 2H), 1.74-1.45 (m, 4H), 1.38 (s, 9H), 1.18 (s, 3H).

### Example 4.10B

### tert-Butyl 2-[5-methoxy-2-oxo-4-(4,4,5,5-tetramethyl-1,3,2-dioxaborolan-2-yl)pyridin-1(2H)-yl]-4-[(1-methylcyclobutyl)oxy]butanoate (Racemate)

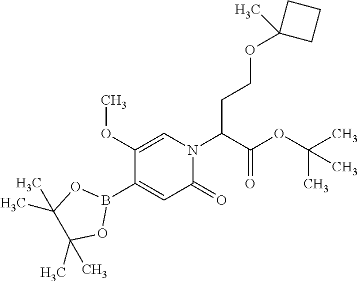 [SMILES: COc1cn(C(CCOC2(C)CCC2)C(=O)OC(C)(C)C)c(=O)cc1B1OC(C)(C)C(C)(C)O1]

To an initial charge of 1.35 g (3.14 mmol) of tert-butyl 2-(4-bromo-5-methoxy-2-oxopyridin-1(2H)-yl)-4-[(1-methylcyclobutyl)oxy]butanoate (racemate), 0.88 g (3.45 mmol) of bis(pinacolato)diboron and 0.92 g (9.41 mmol) of potassium acetate in 38.6 ml of dioxane under argon was added 0.077 g (0.09 mmol) of [1,1-bis(diphenylphosphino)ferrocene]dichloropalladium dichloromethane complex, and the mixture was stirred at 80° C. for 3 h and left to stand overnight. The reaction mixture was cooled, filtered through kieselguhr and washed through three times with dioxane. The filtrate was concentrated and dried under high vacuum. The crude product obtained in this manner was reacted without further purification. Yield: 2.23 mg (56% purity, quantitative).

LC/MS [Method 1]: Rₜ=0.91 min; MS (ESIpos): m/z=395 (M+H)⁺.

### Example 5.1A

### tert-Butyl {4-[5-chloro-2-(2,2,2-trifluoroethoxy)phenyl]-5-methoxy-2-oxopyridin-1(2H)-yl}acetate

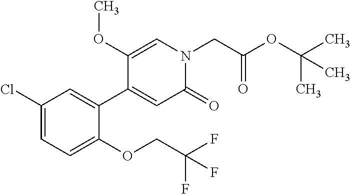 [SMILES: COc1cn(CC(=O)OC(C)(C)C)c(=O)cc1-c1cc(Cl)ccc1OCC(F)(F)F]

To 1.8 g (6.22 mmol) of 2-bromo-4-chlorophenyl-2,2,2-trifluoroethyl ether, 3.66 g (6.22 mmol, 62% purity, 1 eq.) of tert-butyl [5-methoxy-2-oxo-4-(4,4,5,5-tetramethyl-1,3,2-dioxaborolan-2-yl)-pyridin-1(2H)-yl]acetate and 2.58 g (18.7 mmol, 3 eq.) of potassium carbonate were added 5.5 ml of dioxane. Argon was passed through the reaction mixture for 5 min. 152 mg (0.187 mmol, 0.03 eq) of [1,1-bis(diphenylphosphino)ferrocene]dichloropalladium dichloromethane complex were then added, and the mixture was stirred at 80° C. overnight. The reaction mixture was filtered through kieselguhr and washed with dichloromethane and acetonitrile, and the filtrate was concentrated. The crude product was purified by normal phase chromatography (eluent: cyclohexane/ethyl acetate, 0-50%). The product was dissolved in ethyl acetate and the organic phase was washed twice with saturated aqueous sodium bicarbonate solution, then washed with saturated aqueous sodium chloride solution, dried (sodium sulfate) and concentrated. This product was purified by preparative HPLC. Yield: 800 mg (29% of theory).

LC/MS [Method 10]: Rₜ=1.98 min; MS (ESIpos): m/z=448 (M+H)⁺,

¹H-NMR (500 MHz, DMSO-d₆): δ [ppm]=7.49 (dd, 1H), 7.38 (s, 1H), 7.30 (d, 1H), 7.26 (d, 1H), 6.30 (s, 1H), 4.76 (q, 2H), 4.56 (s, 2H), 3.54 (s, 3H), 1.43 (s, 9H).

### Example 5.2A

### tert-Butyl 2-{4-[5-chloro-2-(2,2,2-trifluoroethoxy)phenyl]-5-methoxy-2-oxopyridin-1(2H)-yl}-4-methoxybutanoate (Racemate)

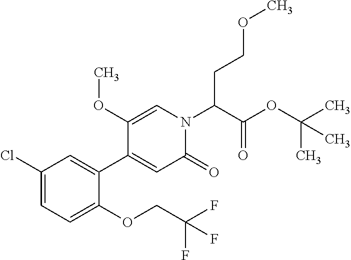 [SMILES: COCCC(C(=O)OC(C)(C)C)n1cc(OC)c(-c2cc(Cl)ccc2OCC(F)(F)F)cc1=O]

To 240 mg (0.83 mmol) of 2-bromo-4-chlorophenyl-2,2,2-trifluoroethyl ether, 532 mg (0.83 mmol, 66% purity, 1 eq.) of tert-butyl 4-methoxy-2-[5-methoxy-2-oxo-4-(4,4,5,5-tetramethyl-1,3,2-dioxaborolan-2-yl)pyridin-1(2H)-yl]butanoate (racemate) and 344 mg (2.49 mmol, 3 eq.) of potassium carbonate were added 8.4 ml of dioxane. Argon was passed through the reaction mixture for 5 min. 20 mg (25 μmol, 0.03 eq.) of [1,1-bis(diphenylphosphino)ferrocene]dichloropalladium dichloromethane complex were then added, and the mixture was stirred at 80° C. for 7 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-45%). Yield: 412 mg (75% purity, 74% of theory).

LC/MS [Method 10]: Rₜ=2.12 min; MS (ESIpos): m/z=506 (M+H)⁺,

### Example 5.2B

### 2-{4-[5-Chloro-2-(2,2,2-trifluoroethoxy)phenyl]-5-methoxy-2-oxopyridin-1(2H)-yl}-4-methoxybutanoic acid (Racemate)

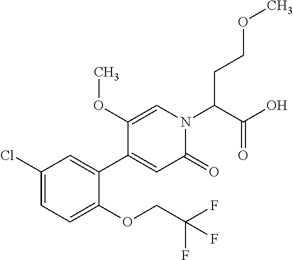 [SMILES: COCCC(C(=O)O)n1cc(OC)c(-c2cc(Cl)ccc2OCC(F)(F)F)cc1=O]

550 mg (0.815 mmol, 75% purity) of tert-butyl 2-{4-[5-chloro-2-(2,2,2-trifluoroethoxy)phenyl]-5-methoxy-2-oxopyridin-1(2H)-yl}-4-methoxybutanoate (racemate) in 9 ml of hydrogen chloride in dioxane (4 M) were reacted according to General Method 6D. Yield: 445 mg (75% purity, 91% of theory)

LC/MS [Method 10]: Rₜ=1.58 min; MS (ESIpos): m/z=450 (M+H)⁺,

### Example 5.2C

### Methyl 4-[(2-{4-[5-chloro-2-(2,2,2-trifluoroethoxy)phenyl]-5-methoxy-2-oxopyridin-1(2H)-yl}-4-methoxybutanoyl)amino]benzoate (Racemate)

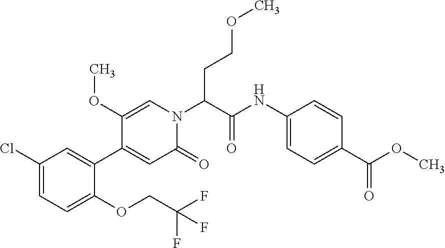 [SMILES: COCCC(C(=O)Nc1ccc(C(=O)OC)cc1)n1cc(OC)c(-c2cc(Cl)ccc2OCC(F)(F)F)cc1=O]

145 mg (0.24 mmol, 75% purity) of 2-{4-[5-chloro-2-(2,2,2-trifluoroethoxy)phenyl]-5-methoxy-2-oxopyridin-1(2H)-yl}-4-methoxybutanoic acid (racemate) and 54.8 mg (0.363 mmol) of methyl 4-aminobenzoate in 2.1 ml of pyridine were reacted according to General Method 5A. Yield: 123 mg (87% of theory).

LC/MS [Method 10]: Rₜ=2.05 min; MS (ESIpos): m/z=583 (M+H)⁺,

¹H NMR (400 MHz, DMSO-d₆): δ [ppm]=10.73 (s, 1H), 7.96-7.90 (m, 2H), 7.81-7.75 (m, 2H), 7.49 (dd, 1H), 7.36-7.31 (m, 2H), 7.26 (d, 1H), 6.32 (s, 1H), 5.76-5.70 (m, 1H), 4.81-4.70 (m, 2H), 3.83 (s, 3H), 3.60 (s, 3H), 3.40-3.33 (m, 1H), 3.26-3.20 (m, 1H), 3.19 (s, 3H), 2.43-2.34 (m, 2H).

### Example 5.3A

### tert-Butyl 2-{4-[5-chloro-2-(2,2,2-trifluoroethoxy)phenyl]-5-methoxy-2-oxopyridin-1(2H)-yl}-3-(1,4-dioxan-2-yl)propanoate (Diastereomer Mixture)

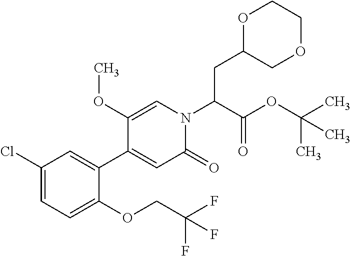 [SMILES: COc1cn(C(CC2COCCO2)C(=O)OC(C)(C)C)c(=O)cc1-c1cc(Cl)ccc1OCC(F)(F)F]

To 186 mg (0.64 mmol) of 2-bromo-4-chlorophenyl-2,2,2-trifluoroethyl ether, 565 mg (0.64 mmol, 53% purity, 1 eq.) of tert-butyl 3-(1,4-dioxan-2-yl)-2-[5-methoxy-2-oxo-4-(4,4,5,5-tetramethyl-1,3,2-dioxaborolan-2-yl)pyridin-1(2H)-yl]propanoate (diastereomer mixture) and 267 mg (1.93 mmol, 3 eq.) of potassium carbonate were added 6.5 ml of dioxane. Argon was passed through the reaction mixture for 5 min. 15.7 mg (19 μmol, 0.03 eq.) of [1,1-bis(diphenylphosphino)ferrocene]dichloropalladium dichloromethane complex were then added, and the mixture was stirred at 80° C. for 16 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-50%). This product was purified by preparative HPLC. Yield: 150 mg (42% of theory).

LC/MS [Method 10]: Rₜ=2.07 min; MS (ESIpos): m/z=548 (M+H)⁺,

¹H-NMR (400 MHz, DMSO-d₆): δ [ppm]=7.49 (dd, 1H), 7.35 (d, 1H), 7.24 (d, 1H), 7.17 (s, 1H), 6.28 (s, 1H), 5.06-4.96 (m, 1H), 4.73 (q, 2H), 3.77-3.69 (d, 1H), 3.63-3.52 (m, 5H), 3.50-3.37 (m, 2H), 3.26-3.17 (m, 1H), 3.15-3.05 (m, 1H), 2.33-2.23 (m, 1H), 2.04-1.94 (m, 1H), 1.38 (s, 9H).

### Example 5.3B

### 2-{4-[5-Chloro-2-(2,2,2-trifluoroethoxy)phenyl]-5-methoxy-2-oxopyridin-1(2H)-yl}-3-(1,4-dioxan-2-yl)propanoic acid (Diastereomer Mixture)

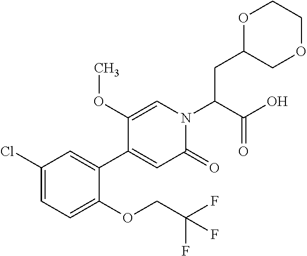 [SMILES: COc1cn(C(CC2COCCO2)C(=O)O)c(=O)cc1-c1cc(Cl)ccc1OCC(F)(F)F]

150 mg (0.274 mmol) of tert-butyl 2-{4-[5-chloro-2-(2,2,2-trifluoroethoxy)phenyl]-5-methoxy-2-oxopyridin-1(2H)-yl}-3-(1,4-dioxan-2-yl)propanoate (diastereomer mixture) in 10 ml of hydrogen chloride in dioxane (4 M) were reacted according to General Method 6D. Yield: 134 mg (100% of theory)

LC/MS [Method 10]: Rₜ=1.57 min; MS (ESIpos): m/z=492 (M+H)⁺.

### Example 5.3C

### Methyl 4-{[2-{4-[5-chloro-2-(2,2,2-trifluoroethoxy)phenyl]-5-methoxy-2-oxopyridin-1(2H)-yl}-3-(1,4-dioxan-2-yl)propanoyl]amino}benzoate (Diastereomer Mixture)

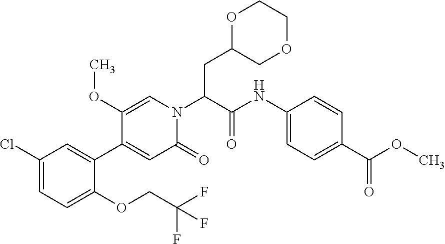 [SMILES: COC(=O)c1ccc(NC(=O)C(CC2COCCO2)n2cc(OC)c(-c3cc(Cl)ccc3OCC(F)(F)F)cc2=O)cc1]

54 mg (0.110 mmol) of 2-{4-[5-chloro-2-(2,2,2-trifluoroethoxy)phenyl]-5-methoxy-2-oxopyridin-1(2H)-yl}-3-(1,4-dioxan-2-yl)propanoic acid (diastereomer mixture) and 25 mg (0.165 mmol) of methyl 4-aminobenzoate in 1 ml of pyridine were reacted according to General Method 5A. Yield: 57 mg (83% of theory).

LC/MS [Method 11]: Rₜ=1.05/1.06 min; MS (ESIpos): m/z=625/625 (M+H)⁺,

### Example 5.4A

### tert-Butyl 2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoate (Diastereomer Mixture)

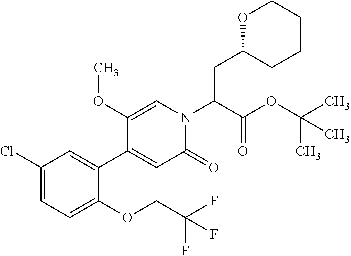 [SMILES: COc1cn(C(C[C@H]2CCCCO2)C(=O)OC(C)(C)C)c(=O)cc1-c1cc(Cl)ccc1OCC(F)(F)F]

To a solution of 330 mg (0.737 mmol) of tert-butyl {4-[5-chloro-2-(2,2,2-trifluoroethoxy)phenyl]-5-methoxy-2-oxopyridin-1(2H)-yl}acetate in 10 ml of tetrahydrofuran under argon at −70° C. was added dropwise 0.995 ml (1.0 M in THF, 1.35 eq.) of lithium bis(trimethylsilyl)amide, and the mixture was stirred for 20 min. Subsequently, 215 mg (0.85 mmol, 1.15 eq.) of (2R)-tetrahydro-2H-pyran-2-ylmethyl trifluoromethanesulfonate were added dropwise, and the mixture was stirred at −70° C. for 15 min and at RT for 1 h. The reaction mixture was cooled to −70° C. again, 0.5 ml (1.0M in THF) of bis(trimethylsilyl)lithium amide was added dropwise followed, after 15 min, by 155 mg (0.42 mmol) of (2R)-tetrahydro-2H-pyran-2-ylmethyl trifluoromethanesulfonate, and the mixture was stirred at −70° C. for 15 min and at RT for 2.5 h. First 20 ml of saturated aqueous ammonium chloride solution and then 20 ml of water and 60 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-45%). Yield 137 mg (70% purity, 24% of theory)

LC/MS [Method 1]: Rₜ=1.22 min; MS (ESIpos): m/z=546 (M+H)⁺.

### Example 5.4B

### 2-{4-[5-Chloro-2-(2,2,2-trifluoroethoxy)phenyl]-5-methoxy-2-oxopyridin-1(2H)-yl}-3-[(2R)-tetrahydro-2H-pyran-2-yl]propanoic acid (Diastereomer Mixture)

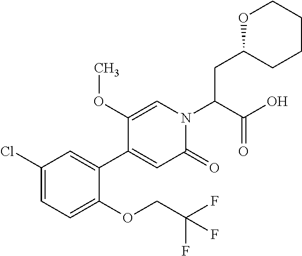 [SMILES: COc1cn(C(C[C@H]2CCCCO2)C(=O)O)c(=O)cc1-c1cc(Cl)ccc1OCC(F)(F)F]

245 mg (0.359 mmol, 80% purity) of tert-butyl 2-{4-[5-chloro-2-(2,2,2-trifluoroethoxy)phenyl]-5-methoxy-2-oxopyridin-1 (2H)-yl}-3-[(2R)-tetrahydro-2H-pyran-2-yl]propanoate (diastereomer mixture) in 5 ml of hydrogen chloride in dioxane (4 M) were reacted according to General Method 6D. The crude product was purified by preparative HPLC. Yield: 110 mg (63% of theory)

LC/MS [Method 10]: Rₜ=1.79/1.81 min; MS (ESIpos): m/z=490/490 (M+H)⁺.

### Example 5.4C

### Methyl 4-[(2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoyl)amino]benzoate (Diastereomer Mixture)

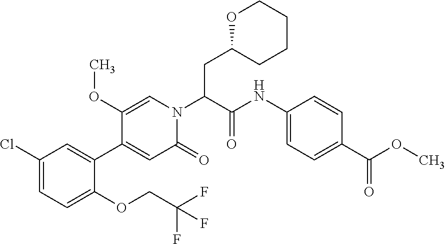 [SMILES: COC(=O)c1ccc(NC(=O)C(C[C@H]2CCCCO2)n2cc(OC)c(-c3cc(Cl)ccc3OCC(F)(F)F)cc2=O)cc1]

35 mg (71 μmol) of 2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoic acid (diastereomer mixture) and 16.5 mg (107 μmol) of methyl 4-aminobenzoate in 1 ml of pyridine were reacted according to General Method 5A. Yield: 39 mg (88% of theory).

LC/MS [Method 10]: Rₜ=2.21/2.24 min; MS (ESIpos): m/z=623/623 (M+H)⁺.

### Example 5.5A

### tert-Butyl 2-{4-[5-chloro-2-(2,2,2-trifluoroethoxy)phenyl]-5-methoxy-2-oxopyridin-1(2H)-yl}-3-(1-methyl-1H-pyrazol-3-yl)propanoate (Racemate)

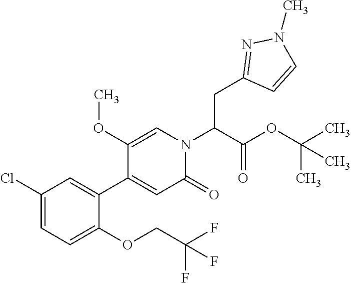 [SMILES: COc1cn(C(Cc2ccn(C)n2)C(=O)OC(C)(C)C)c(=O)cc1-c1cc(Cl)ccc1OCC(F)(F)F]

To a solution of 510 mg (1.12 mmol) of tert-butyl {4-[5-chloro-2-(2,2,2-trifluoroethoxy)phenyl]-5-methoxy-2-oxopyridin-1(2H)-yl}acetate in 8.4 ml of tetrahydrofuran under argon at −70° C. were added dropwise 1.4 ml (1.0 M in THF, 1.25 eq.) of lithium bis(trimethylsilyl)amide, and the mixture was stirred for 20 min. Subsequently, 251 mg (about 1.5 mmol) of a 1:1 mixture of 3-(bromomethyl)-1-methyl-1H-pyrazole and 3-(chloromethyl)-1-methyl-1H-pyrazole were added and the mixture was stirred at −70° C. for 15 min and at RT for 90 min. First 15 ml of saturated aqueous ammonium chloride solution and then 15 ml of water and 60 ml of ethyl acetate were added to the reaction mixture. After phase separation, the organic phase was washed with saturated aqueous sodium chloride solution, dried (sodium sulfate) and concentrated. The crude product was purified by normal phase chromatography (eluent: cyclohexane/ethyl acetate, 0-80%). Yield: 340 mg (56% of theory)

LC/MS [Method 10]: Rₜ=1.99 min; MS (ESIpos): m/z=542 (M+H)⁺,

¹H-NMR (500 MHz, DMSO-d₆): δ [ppm]=7.50-7.46 (m, 2H), 7.28 (d, 1H), 7.23 (d, 1H), 7.14 (s, 1H), 6.25 (s, 1H), 5.89 (d, 1H), 5.26 (dd, 1H), 4.72 (q, 2H), 3.73 (s, 3H), 3.47 (s, 3H), 3.43-3.35 (m, 1H), 3.34-3.27 (m, 1H), 1.39 (s, 9H).

### Example 5.5B

### 2-{4-[5-Chloro-2-(2,2,2-trifluoroethoxy)phenyl]-5-methoxy-2-oxopyridin-1(2H)-yl}-3-(1-methyl-1 H-pyrazol-3-yl)propanoic acid (Racemate)

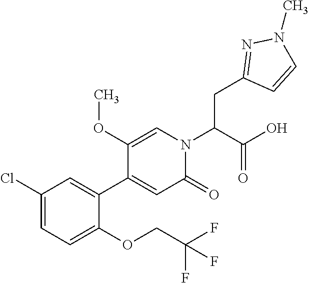 [SMILES: COc1cn(C(Cc2ccn(C)n2)C(=O)O)c(=O)cc1-c1cc(Cl)ccc1OCC(F)(F)F]

340 mg (0.62 mmol) of tert-butyl 2-{4-[5-chloro-2-(2,2,2-trifluoroethoxy)phenyl]-5-methoxy-2-oxopyridin-1(2H)-yl}-3-(1-methyl-1H-pyrazol-3-yl)propanoate (racemate) in 7 ml of hydrogen chloride in dioxane (4 M) were reacted according to General Method 6D. Yield: 320 mg (97% of theory)

LC/MS [Method 10]: Rₜ=1.52 min; MS (ESIpos): m/z=486 (M+H)⁺.

### Example 5.5C

### Methyl 4-{[2-{4-[5-chloro-2-(2,2,2-trifluoroethoxy)phenyl]-5-methoxy-2-oxopyridin-1(2H)-yl}-3-(1-methyl-1H-pyrazol-3-yl)propanoyl]amino}benzoate (Racemate)

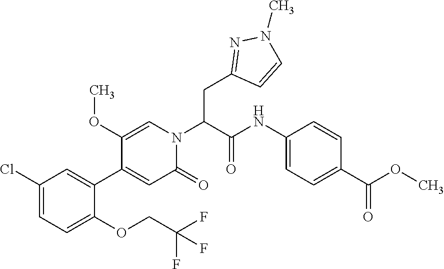 [SMILES: COC(=O)c1ccc(NC(=O)C(Cc2ccn(C)n2)n2cc(OC)c(-c3cc(Cl)ccc3OCC(F)(F)F)cc2=O)cc1]

125 mg (235 μmol) of 2-{4-[5-chloro-2-(2,2,2-trifluoroethoxy)phenyl]-5-methoxy-2-oxopyridin-1(2H)-yl}-3-(1-methyl-1H-pyrazol-3-yl)propanoic acid (racemate) and 54 mg (352 μmol) of methyl 4-aminobenzoate in 3.1 ml of pyridine were reacted according to General Method 5A. Yield: 131 mg (90% of theory).

LC/MS [Method 1]: Rₜ=1.04 min; MS (ESIpos): m/z=619 (M+H)⁺,

¹H-NMR (400 MHz, DMSO-d₆): δ [ppm]=10.85 (s, 1H), 7.98-7.90 (m, 2H), 7.82-7.75 (m, 2H), 7.52-7.45 (m, 2H), 7.43 (s, 1H), 7.29 (d, 1H), 7.25 (d, 1H), 6.26 (s, 1H), 5.95-5.85 (m, 2H), 4.82-4.66 (m, 2H), 3.83 (s, 3H), 3.73 (s, 3H), 3.59 (s, 3H), 3.52-3.36 (m, 2H).

### Example 5.6A

### tert-Butyl 4-tert-butoxy-2-{4-[5-chloro-2-(2,2,2-trifluoroethoxy)phenyl]-5-methoxy-2-oxopyridin-1(2H)-yl}butanoate (Racemate)

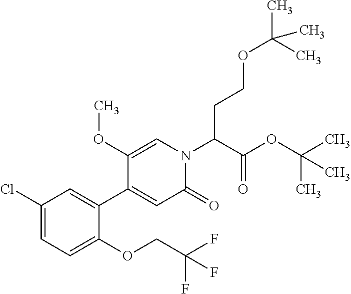 [SMILES: COc1cn(C(CCOC(C)(C)C)C(=O)OC(C)(C)C)c(=O)cc1-c1cc(Cl)ccc1OCC(F)(F)F]

To 425 mg (1.47 mmol) of 2-bromo-4-chlorophenyl-2,2,2-trifluoroethyl ether, 1.0 g (1.47 mmol, 68% purity, 1 eq.) of tert-butyl 4-tert-butoxy-2-[5-methoxy-2-oxo-4-(4,4,5,5-tetramethyl-1,3,2-dioxaborolan-2-yl)pyridin-1(2H)-yl]butanoate (racemate) and 609 mg (4.41 mmol, 3 eq.) of potassium carbonate were added 15.5 ml of dioxane. Argon was passed through the reaction mixture for 5 min. 36 mg (44 μmol, 0.03 eq.) of [1,1-bis(diphenylphosphino)ferrocene]dichloropalladium dichloromethane complex were then added, and the mixture was stirred at 80° C. for 16 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-50%). Yield: 480 mg (60% of theory).

LC/MS [Method 10]: Rₜ=2.37 min; MS (ESIpos): m/z=548 (M+H)⁺.

### Example 5.6B

### 4-tert-Butoxy-2-{4-[5-chloro-2-(2,2,2-trifluoroethoxy)phenyl]-5-methoxy-2-oxopyridin-1(2H)-yl}butanoic acid (Racemate)

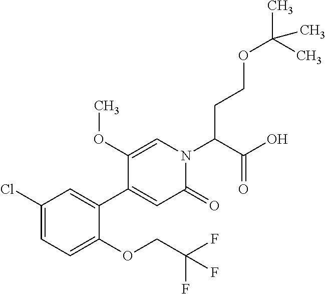 [SMILES: COc1cn(C(CCOC(C)(C)C)C(=O)O)c(=O)cc1-c1cc(Cl)ccc1OCC(F)(F)F]

480 mg (0.876 mmol) of tert-butyl 4-tert-butoxy-2-{4-[5-chloro-2-(2,2,2-trifluoroethoxy)phenyl]-5-methoxy-2-oxopyridin-1(2H)-yl}butanoate (racemate) were reacted according to General Method 6C. Yield: 439 mg (99% of theory)

LC/MS [Method 1]: Rₜ=1.00 min; MS (ESIpos): m/z=492 (M+H)⁺.

### Example 5.6C

### Methyl 4-[(4-tert-butoxy-2-{4-[5-chloro-2-(2,2,2-trifluoroethoxy)phenyl]-5-methoxy-2-oxopyridin-1 (2H)-yl}butanoyl)amino]benzoate (Racemate)

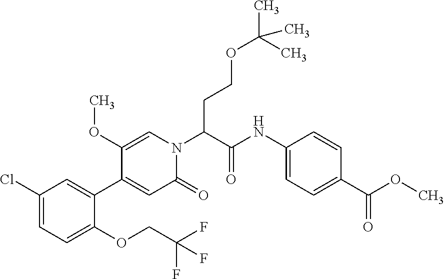 [SMILES: COC(=O)c1ccc(NC(=O)C(CCOC(C)(C)C)n2cc(OC)c(-c3cc(Cl)ccc3OCC(F)(F)F)cc2=O)cc1]

150 mg (0.296 mmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 68 mg (0.444 mmol) of methyl 4-aminobenzoate in 2 ml of pyridine were reacted according to General Method 5A. Yield: 167 mg (90% of theory).

LC/MS [Method 10]: Rₜ=2.29 min; MS (ESIpos): m/z=625 (M+H)⁺,

¹H-NMR (400 MHz, DMSO-d₆): δ [ppm]=10.74 (s, 1H), 7.96-7.89 (m, 2H), 7.84-7.76 (m, 2H), 7.49 (dd, 1H), 7.32 (s, 1H), 7.30 (d, 1H), 7.26 (d, 1H), 6.32 (s, 1H), 5.76 (dd, 1H), 4.85-4.65 (m, 2H), 3.82 (s, 3H), 3.60 (s, 3H), 3.43-3.34 (m, 1H), 3.28-3.19 (m, 1H), 2.42-2.29 (m, 2H), 1.03 (s, 9H).

### Example 5.7A

### tert-Butyl 2-{4-[5-chloro-2-(2,2,2-trifluoroethoxy)phenyl]-5-methoxy-2-oxopyridin-1(2H)-yl}-3-(5,6-dihydro-4H-1,2-oxazin-3-yl)propanoate (Racemate)

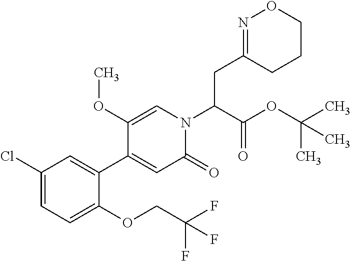 [SMILES: COc1cn(C(CC2=NOCCC2)C(=O)OC(C)(C)C)c(=O)cc1-c1cc(Cl)ccc1OCC(F)(F)F]

To 110 mg (0.38 mmol) of 2-bromo-4-chlorophenyl-2,2,2-trifluoroethyl ether, 360 mg (0.38 mmol, 49% purity, 1 eq.) of tert-butyl 3-(5,6-dihydro-4H-1,2-oxazin-3-yl)-2-[5-methoxy-2-oxo-4-(4,4,5,5-tetramethyl-1,3,2-dioxaborolan-2-yl)pyridin-1(2H)-yl]propanoate (racemate) and 158 mg (1.14 mmol, 3 eq.) of potassium carbonate were added 3.9 ml of dioxane. Argon was passed through the reaction mixture for 5 min. 9.3 mg (11 μmol, 0.03 eq.) of [1,1-bis(diphenylphosphino)ferrocene]dichloropalladium dichloromethane complex were then added, and the mixture was stirred at 80° C. for 16 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-50%). Yield: 20 mg (70% purity, 7% of theory).

LC/MS [Method 10]: Rₜ=2.05 min; MS (ESIpos): m/z=545 (M+H)⁺,

### Example 5.7B

### 2-{4-[5-Chloro-2-(2,2,2-trifluoroethoxy)phenyl]-5-methoxy-2-oxopyridin-1(2H)-yl}-3-(5,6-dihydro-4H-1,2-oxazin-3-yl)propanoic acid (Racemate)

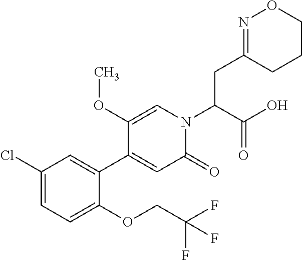 [SMILES: COc1cn(C(CC2=NOCCC2)C(=O)O)c(=O)cc1-c1cc(Cl)ccc1OCC(F)(F)F]

20 mg (70% purity, 25.7 μmol) of tert-butyl 2-{4-[5-chloro-2-(2,2,2-trifluoroethoxy)phenyl]-5-methoxy-2-oxopyridin-1(2H)-yl}-3-(5,6-dihydro-4H-1,2-oxazin-3-yl)propanoate (racemate) were reacted according to General Method 6D. Yield: 18 mg (70% purity, quant.).

LC/MS [Method 10]: Rₜ=1.56 min; MS (ESIpos): m/z=489 (M+H)⁺.

### Example 5.8A

### tert-Butyl 4-tert-butoxy-2-{4-[5-chloro-2-(2,2-difluoroethoxy)phenyl]-5-methoxy-2-oxopyridin-1(2H)-yl}butanoate (Racemate)

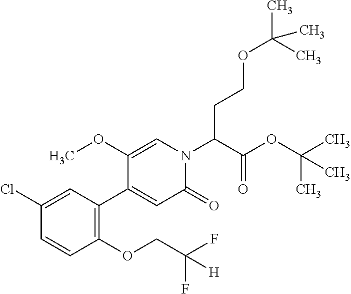 [SMILES: COc1cn(C(CCOC(C)(C)C)C(=O)OC(C)(C)C)c(=O)cc1-c1cc(Cl)ccc1OCC(F)F]

129 mg (0.475 mmol, 1 eq.) of 2-bromo-4-chloro-1-(2,2-difluoroethoxy)benzene, 325 mg (68% purity, 0.475 mmol) of tert-butyl 4-tert-butoxy-2-[5-methoxy-2-oxo-4-(4,4,5,5-tetramethyl-1,3,2-dioxaborolan-2-yl)pyridin-1(2H)-yl]butanoate (racemate) and 197 mg (1.42 mmol, 3.0 eq.) of potassium carbonate were initially charged in 5.0 ml of dioxane. Argon was passed through the reaction mixture for 5 min, and then 11.6 mg (14.2 μmol, 0.03 eq.) of [1,1-bis(diphenylphosphino)ferrocene]dichloropalladium dichloromethane complex were added and the mixture was agitated at 80° C. for 6 h. The reaction mixture was filtered through kieselguhr and washed with dichloromethane and acetonitrile, and the filtrate was concentrated. The crude product was purified by normal phase chromatography (eluent: cyclohexane/ethyl acetate 10:1 to 2:1). Yield: 197 mg (76% of theory)

LC/MS [Method 10]: Rₜ=2.32 min; MS (ESIpos): m/z=530 (M+H)⁺,

¹H-NMR (400 MHz, DMSO-d₆): δ [ppm]=7.45 (dd, 1H), 7.26 (d, 1H), 7.22-7.17 (m, 2H), 6.27 (s, 1H), 6.17 (tt, 1H), 5.15-5.08 (m, 1H), 4.35-4.25 (m, 2H), 3.57 (s, 3H), 3.38-3.33 (m, 1H, partly concealed), 3.18-3.10 (m, 1H), 2.33-2.18 (m, 2H), 1.40 (s, 9H), 1.07 (s, 9H).

### Example 5.8B

### 4-tert-Butoxy-2-{4-[5-chloro-2-(2,2-difluoroethoxy)phenyl]-5-methoxy-2-oxopyridin-1(2H)-yl}butanoic acid (Racemate)

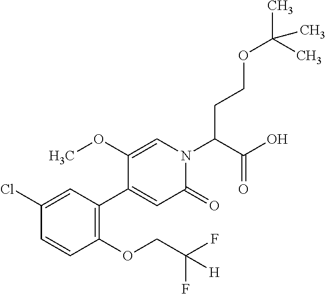 [SMILES: COc1cn(C(CCOC(C)(C)C)C(=O)O)c(=O)cc1-c1cc(Cl)ccc1OCC(F)F]

197 mg (0.372 mmol) of tert-butyl 4-tert-butoxy-2-{4-[5-chloro-2-(2,2-difluoroethoxy)phenyl]-5-methoxy-2-oxopyridin-1(2H)-yl}butanoate (racemate) in 39 ml of ethanol/tetrahydrofuran (2:1) mixture were reacted according to General Method 6C. Yield: 134 mg (76% of theory).

LC/MS [Method 10]: Rₜ=1.84 min; MS (ESIpos): m/z=474 (M+H)⁺,

¹H-NMR (400 MHz, DMSO-d₆): δ [ppm]=12.91 (brs, 1H), 7.45 (dd, 1H), 7.28-7.19 (m, 3H), 6.26 (s, 1H), 6.20 (tt, 1H), 5.18-5.11 (m, 1H), 4.31 (td, 2H), 3.56 (s, 3H), 3.18-3.10 (m, 1H), 2.34-2.18 (m, 2H), 1.06 (s, 9H).

### Example 5.8C

### Methyl 4-[(4-tert-butoxy-2-{4-[5-chloro-2-(2,2-difluoroethoxy)phenyl]-5-methoxy-2-oxopyridin-1 (2H)-yl}butanoyl)amino]benzoate (Racemate)

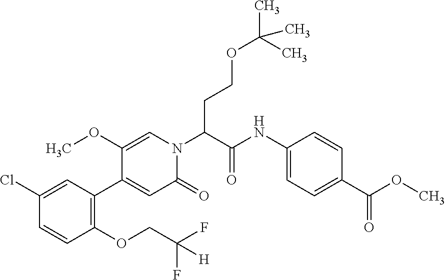 [SMILES: COC(=O)c1ccc(NC(=O)C(CCOC(C)(C)C)n2cc(OC)c(-c3cc(Cl)ccc3OCC(F)F)cc2=O)cc1]

40 mg (0.084 mmol) of 4-tert-butoxy-2-{4-[5-chloro-2-(2,2-difluoroethoxy)phenyl]-5-methoxy-2-oxopyridin-1(2H)-yl}butanoic acid (racemate) and 19 mg (0.13 mmol) of methyl 4-aminobenzoate in 0.5 ml of pyridine were reacted according to General Method 5A. Yield: 48 mg (94% of theory).

LC/MS [Method 10]: Rₜ=2.23 min; MS (ESIpos): m/z=607 (M+H)⁺,

¹H-NMR (400 MHz, DMSO-d₆): δ [ppm]=10.75 (s, 1H), 7.96-7.91 (m, 2H), 7.83-7.78 (m, 2H), 7.48-7.44 (m, 1H), 7.34 (s, 1H), 7.27-7.21 (m, 2H), 6.33 (s, 1H), 6.21 (tt, 1H), 5.79-5.74 (m, 1H), 4.41-4.25 (m, 2H), 3.83 (s, 3H), 3.62 (s, 3H), 3.42-3.46 (m, 1H), 3.30-3.24 (m, 1H, partly concealed), 2.39-2.31 (m, 2H), 1.04 (s, 9H).

### Example 5.9A

### tert-Butyl 2-{4-[5-chloro-2-(2,2-difluoroethoxy)phenyl]-5-methoxy-2-oxopyridin-1(2H)-yl}-3-(1,4-dioxan-2-yl)propanoate (Diastereomer Mixture)

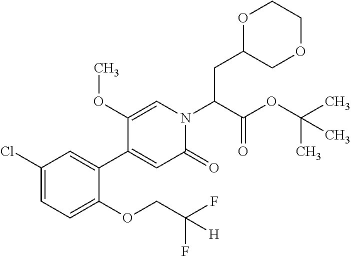 [SMILES: COc1cn(C(CC2COCCO2)C(=O)OC(C)(C)C)c(=O)cc1-c1cc(Cl)ccc1OCC(F)F]

To 285 mg (1.05 mmol) of 2-bromo-4-chloro-1-(2,2-difluoroethoxy)benzene, 920 mg (1.05 mmol, 53% purity, 1 eq.) of tert-butyl 3-(1,4-dioxan-2-yl)-2-[5-methoxy-2-oxo-4-(4,4,5,5-tetramethyl-1,3,2-dioxaborolan-2-yl)pyridin-1(2H)-yl]propanoate (diastereomer mixture) and 434 mg (3.14 mmol, 3 eq.) of potassium carbonate were added 10.6 ml of dioxane. Argon was passed through the reaction mixture for 5 min. 51 mg (63 μmol, 0.06 eq.) of [1,1-bis(diphenylphosphino)ferrocene]dichloropalladium dichloromethane complex were then added, and the mixture was stirred at 80° C. for 18 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-50%). This product was purified by preparative HPLC. Yield: 395 mg (80% purity, 57% of theory).

LC/MS [Method 11]: Rₜ=1.10 min; MS (ESIpos): m/z=530 (M+H)⁺,

### Example 5.9B

### 2-{4-[5-Chloro-2-(2,2-difluoroethoxy)phenyl]-5-methoxy-2-oxopyridin-1(2H)-yl}-3-(1,4-dioxan-2-yl)propanoic acid (Diastereomer Mixture)

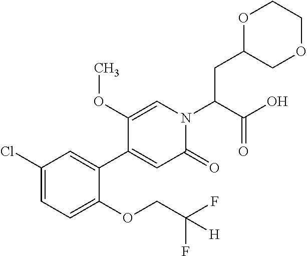 [SMILES: COc1cn(C(CC2COCCO2)C(=O)O)c(=O)cc1-c1cc(Cl)ccc1OCC(F)F]

385 mg (80% purity, 0.596 mmol) of tert-butyl 2-{4-[5-chloro-2-(2,2-difluoroethoxy)phenyl]-5-methoxy-2-oxopyridin-1(2H)-yl}-3-(1,4-dioxan-2-yl)propanoate (diastereomer mixture) in 21.8 ml of hydrogen chloride in dioxane (4 M) were reacted according to General Method 6D. Yield: 340 mg (80% purity, 96% of theory)

LC/MS [Method 10]: Rₜ=1.48 min; MS (ESIpos): m/z=474 (M+H)⁺.

### Example 5.9C

### Methyl 4-{[2-{4-[5-chloro-2-(2,2-difluoroethoxy)phenyl]-5-methoxy-2-oxopyridin-1(2H)-yl}-3-(1,4-dioxan-2-yl)propanoyl]amino}benzoate (Diastereomer Mixture)

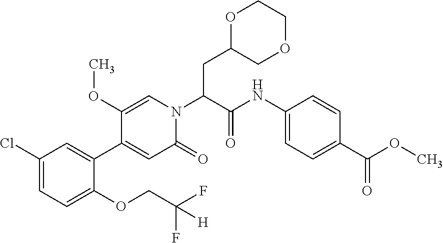 [SMILES: COC(=O)c1ccc(NC(=O)C(CC2COCCO2)n2cc(OC)c(-c3cc(Cl)ccc3OCC(F)F)cc2=O)cc1]

50 mg (80% purity, 0.084 mmol) of tert-butyl 2-{4-[5-chloro-2-(2,2-difluoroethoxy)phenyl]-5-methoxy-2-oxopyridin-1(2H)-yl}-3-(1,4-dioxan-2-yl)propanoate (diastereomer mixture) and 19 mg (0.127 mmol) of methyl 4-aminobenzoate in 0.75 ml of pyridine were reacted according to General Method 5A. Yield: 48 mg (94% of theory).

LC/MS [Method 10]: Rₜ=1.91/1.94 min; MS (ESIpos): m/z=607/607 (M+H)⁺,

### Example 5.10A

### tert-Butyl 2-{4-[5-chloro-2-(2,2,2-trifluoroethoxy)phenyl]-5-methoxy-2-oxopyridin-1(2H)-yl}-4-isopropoxybutanoate (Racemate)

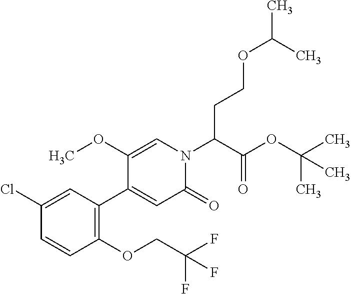 [SMILES: COc1cn(C(CCOC(C)C)C(=O)OC(C)(C)C)c(=O)cc1-c1cc(Cl)ccc1OCC(F)(F)F]

248 mg (0.86 mmol) of 2-bromo-4-chlorophenyl 2,2,2-trifluoroethyl ether, 605 mg (64% purity, 0.86 mmol) of tert-butyl 4-isopropoxy-2-[5-methoxy-2-oxo-4-(4,4,5,5-tetramethyl-1,3,2-dioxaborolan-2-yl)pyridin-1(2H)-yl]butanoate (racemate) and 355 mg (2.57 mmol, 3.0 eq.) of potassium carbonate were initially charged in 5.0 ml of dioxane. Argon was passed through the reaction mixture for 5 min, then 21 mg (0.026 mmol, 0.03 eq.) of [1,1-bis(diphenylphosphino)ferrocene]dichloropalladium dichloromethane complex were added and the mixture was stirred at 80° C. overnight. The reaction mixture was filtered through kieselguhr and washed twice with dichloromethane/acetonitrile mixture (1:1), and the filtrate was concentrated. The crude product was purified by normal phase chromatography (cyclohexane/ethyl acetate gradient). Yield: 368 mg (80% of theory).

LC/MS [Method 1]: Rₜ=2.31 min; MS (ESIpos): m/z=534 (M+H)⁺,

¹H-NMR (400 MHz, DMSO-d₆): δ [ppm]=7.49 (dd, 1H), 7.31 (d, 1H), 7.24 (d, 1H), 7.18 (s, 1H), 6.28 (s, 1H), 5.11-5.04 (m, 1H), 4.73 (q, 2H), 3.55 (s, 3H), 3.46-3.36 (m, 2H), 3.11 (d, 1H), 2.38-2.20 (m, 2H), 1.39 (s, 9H), 1.06-1.00 (m, 6H).

### Example 5.10B

### 2-{4-[5-Chloro-2-(2,2,2-trifluoroethoxy)phenyl]-5-methoxy-2-oxopyridin-1(2H)-yl}-4-isopropoxybutanoic acid (Racemate)

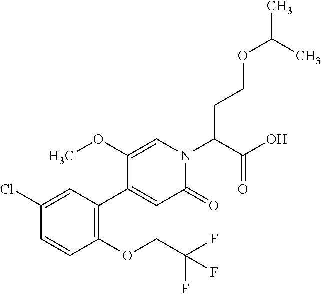 [SMILES: COc1cn(C(CCOC(C)C)C(=O)O)c(=O)cc1-c1cc(Cl)ccc1OCC(F)(F)F]

365 mg (0.68 mmol) of tert-butyl 2-{4-[5-chloro-2-(2,2,2-trifluoroethoxy)phenyl]-5-methoxy-2-oxopyridin-1(2H)-yl}-4-isopropoxybutanoate (racemate) and 3.42 ml (3.42 mmol) of aqueous lithium hydroxide solution (1N) in 28 ml of ethanol/THF (2:1) were reacted according to General Method 6C. Yield: 315 mg (97% of theory).

LC/MS [Method 1]: Rₜ=1.82 min; MS (ESIpos): m/z=478 (M+H)⁺,

¹H-NMR (400 MHz, DMSO-d₆): δ [ppm]=12.88 (br. s., 1H), 7.49 (dd, 1H), 7.32 (d, 1H), 7.28-7.19 (m, 2H), 6.27 (s, 1H), 5.16-5.09 (m, 1H), 4.74 (q, 2H), 3.55 (s, 3H), 3.44-3.35 (m, 2H), 3.12-3.03 (m, 1H), 2.43-2.22 (m, 2H), 1.06-1.00 (m, 6H).

### Example 5.10C

### Methyl 4-[(2-{4-[5-chloro-2-(2,2,2-trifluoroethoxy)phenyl]-5-methoxy-2-oxopyridin-1(2H)-yl}-4-isopropoxybutanoyl)amino]benzoate (Racemate)

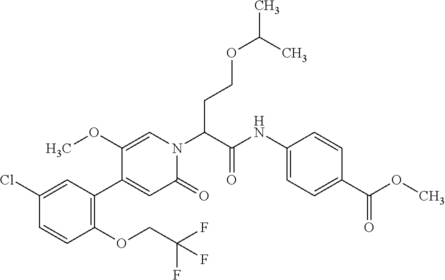 [SMILES: COC(=O)c1ccc(NC(=O)C(CCOC(C)C)n2cc(OC)c(-c3cc(Cl)ccc3OCC(F)(F)F)cc2=O)cc1]

100 mg (0.21 mmol) of 2-{4-[5-chloro-2-(2,2,2-trifluoroethoxy)phenyl]-5-methoxy-2-oxopyridin-1(2H)-yl}-4-isopropoxybutanoic acid (racemate) and 53 mg (89% purity, 0.31 mmol) of methyl 4-aminobenzoate in 1.13 ml of pyridine were reacted according to General Method 5A. Yield: 109 mg (85% of theory).

LC/MS [Method 1]: Rₜ=1.82 min; MS (ESIpos): m/z=478 (M+H)⁺,

¹H-NMR (400 MHz, DMSO-d₆): δ [ppm]=12.88 (br. s., 1H), 7.49 (dd, 1H), 7.32 (d, 1H), 7.28-7.19 (m, 2H), 6.27 (s, 1H), 5.16-5.09 (m, 1H), 4.74 (q, 2H), 3.55 (s, 3H), 3.44-3.35 (m, 2H), 3.12-3.03 (m, 1H), 2.43-2.22 (m, 2H), 1.06-1.00 (m, 6H).

### Example 5.11A

### tert-Butyl 2-{4-[5-chloro-2-(2,2,2-trifluoroethoxy)phenyl]-5-methoxy-2-oxopyridin-1(2H)-yl}-4-(trifluoromethoxy)butanoate (Racemate)

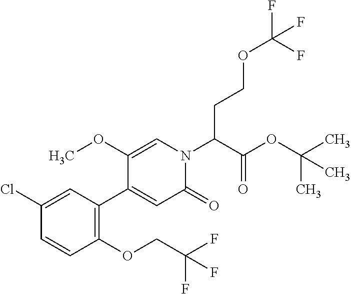 [SMILES: COc1cn(C(CCOC(F)(F)F)C(=O)OC(C)(C)C)c(=O)cc1-c1cc(Cl)ccc1OCC(F)(F)F]

234 mg (0.81 mmol) of 2-bromo-4-chlorophenyl 2,2,2-trifluoroethyl ether, 577 mg (67% purity, 0.81 mmol) of tert-butyl 2-[5-methoxy-2-oxo-4-(4,4,5,5-tetramethyl-1,3,2-dioxaborolan-2-yl)pyridin-1(2H)-yl]-4-(trifluoromethoxy)butanoate (racemate) and 335 mg (2.43 mmol, 3.0 eq.) of potassium carbonate were initially charged in 5.0 ml of dioxane. Argon was passed through the reaction mixture for 5 min, then 20 mg (0.024 mmol, 0.03 eq.) of [1,1-bis(diphenylphosphino)ferrocene]dichloropalladium dichloromethane complex were added and the mixture was stirred at 80° C. overnight. The reaction mixture was filtered through kieselguhr and washed twice with dichloromethane/acetonitrile mixture (1:1), and the filtrate was concentrated. The crude product was purified by normal phase chromatography (cyclohexane/ethyl acetate gradient). Yield: 226 mg (50% of theory).

LC/MS [Method 1]: Rₜ=2.30 min; MS (ESIpos): m/z=560 (M+H)⁺,

¹H-NMR (400 MHz, DMSO-d₆): δ [ppm]=7.49 (dd, 1H), 7.30 (d, 1H), 7.28-7.20 (m, 2H), 6.30 (s, 1H), 5.12-5.03 (m, 1H), 4.72 (q, 2H), 4.19-4.09 (m, 1H), 4.00-3.91 (m, 1H), 3.55 (s, 3H), 2.55-2.45 (m, 2H, partly concealed), 1.39 (s, 9H).

### Example 5.11B

### 2-{4-[5-Chloro-2-(2,2,2-trifluoroethoxy)phenyl]-5-methoxy-2-oxopyridin-1(2H)-yl}-4-(trifluoromethoxy)butanoic acid (Racemate)

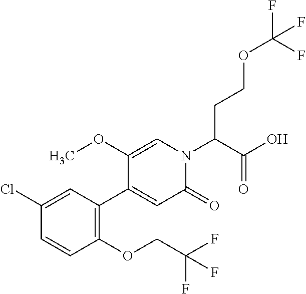 [SMILES: COc1cn(C(CCOC(F)(F)F)C(=O)O)c(=O)cc1-c1cc(Cl)ccc1OCC(F)(F)F]

225 mg (0.40 mmol) of tert-butyl 2-{4-[5-chloro-2-(2,2,2-trifluoroethoxy)phenyl]-5-methoxy-2-oxopyridin-1(2H)-yl}-4-(trifluoromethoxy)butanoate (racemate) and 2.01 ml (2.01 mmol) of aqueous lithium hydroxide solution (1N) in 16 ml of ethanol/THF (2:1) were reacted according to General Method 6C. Yield: 187 mg (89% of theory).

LC/MS [Method 1]: Rₜ=1.86 min; MS (ESIpos): m/z=504 (M+H)⁺,

¹H-NMR (400 MHz, DMSO-d₆): δ [ppm]=13.08 (br. s., 1H), 7.49 (dd, 1H), 7.33-7.29 (m, 2H), 7.25 (d, 1H), 6.29 (s, 1H), 5.17-5.10 (m, 1H), 4.73 (q, 2H), 4.16-4.09 (m, 1H), 3.96-3.87 (m, 1H), 3.54 (s, 3H), 2.58-2.55 (m, 2H, partly concealed).

### Example 5.12A

### tert-Butyl (4S)-2-[4-[5-chloro-2-(2,2,2-trifluoroethoxy)phenyl]-5-methoxy-2-oxo-1-pyridyl]-4-methoxypentanoate (Racemate)

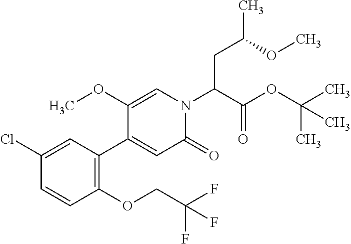 [SMILES: COc1cn(C(C[C@H](C)OC)C(=O)OC(C)(C)C)c(=O)cc1-c1cc(Cl)ccc1OCC(F)(F)F]

0.97 g (3.36 mmol) of 2-bromo-4-chlorophenyl 2,2,2-trifluoroethyl ether, 2.98 g (40% purity, 3.36 mmol) of tert-butyl (4S)-4-methoxy-2-[5-methoxy-2-oxo-4-(4,4,5,5-tetramethyl-1,3-dioxolan-2-yl)-1-pyridyl]pentanoate (racemate) and 1.39 g (10.07 mmol, 3.0 eq.) of potassium carbonate were initially charged in 50.0 ml of dioxane. Argon was passed through the reaction mixture for 5 min, then 82 mg (0.101 mmol, 0.03 eq.) of [1,1-bis(diphenylphosphino)ferrocene]dichloropalladium dichloromethane complex were added and the mixture was stirred at 80° C. overnight. The reaction mixture was filtered through kieselguhr and washed with 100 ml of dioxane, and the filtrate was concentrated. The crude product was purified by normal phase chromatography (cyclohexane/ethyl acetate gradient). Yield: 1.60 g (90% of theory).

LC/MS [Method 1]: Rₜ=1.15 min; MS (ESIpos): m/z=520 (M+H)⁺,

¹H-NMR (400 MHz, DMSO-d₆): δ [ppm]=7.49 (dd, 1H), 7.33 (dd, 1H), 7.26-7.17 (m, 2H), 6.29-6.26 (m, 1H), 5.18-5.06 (m, 1H), 4.78-4.68 (m, 2H), 3.56 (s, 3H), 3.18-3.12 (m, 3H), 2.94-2.84 (m, 1H), 2.39-2.29 (m, 1H), 2.22-2.16 (m, 0.5H), 2.10-2.01 (m, 0.5H), 1.39 (d, 9H), 1.12-1.04 (m, 3H).

### Example 5.12B

### (4S)-2-[4-[5-Chloro-2-(2,2,2-trifluoroethoxy)phenyl]-5-methoxy-2-oxo-1-pyridyl]-4-methoxypentanoic acid (Racemate)

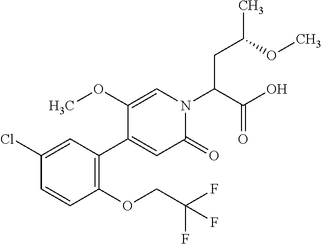 [SMILES: COc1cn(C(C[C@H](C)OC)C(=O)O)c(=O)cc1-c1cc(Cl)ccc1OCC(F)(F)F]

1.60 g (3.08 mmol) of tert-butyl (4S)-2-[4-[5-chloro-2-(2,2,2-trifluoroethoxy)phenyl]-5-methoxy-2-oxo-1-pyridyl]-4-methoxypentanoate (racemate) and 15.39 ml (15.39 mmol) of aqueous lithium hydroxide solution (1N) in 30 ml of ethanol/THF (1:2) were reacted according to General Method 6C. Yield: 1.32 g (91% of theory).

¹H-NMR (400 MHz, DMSO-d₆): δ [ppm]=1.07 (dd, 3H), 2.38-2.04 (m, 2H), 2.93-2.82 (m, 1H), 3.13 (d, 3H), 3.55 (s, 3H), 4.81-4.68 (m, 2H), 5.29-5.08 (m, 1H), 6.28-6.23 (m, 1H), 7.29-7.20 (m, 2H), 7.33 (dd, 1H), 7.52-7.44 (m, 1H).

### Example 5.13A

### tert-Butyl 2-{4-[5-chloro-2-(2-fluoroethoxy)phenyl]-5-methoxy-2-oxopyridin-1(2H)-yl}-3-(1,4-dioxan-2-yl)propanoate (Diastereomer Mixture)

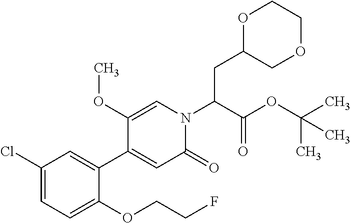 [SMILES: COc1cn(C(CC2COCCO2)C(=O)OC(C)(C)C)c(=O)cc1-c1cc(Cl)ccc1OCCF]

To 305 mg (1.20 mmol) of 2-bromo-4-chloro-1-(2-fluoroethoxy)benzene, 836 mg (1.20 mmol, 67% purity, 1 eq.) of tert-butyl 3-(1,4-dioxan-2-yl)-2-[5-methoxy-2-oxo-4-(4,4,5,5-tetramethyl-1,3,2-dioxaborolan-2-yl)pyridin-1(2H)-yl]propanoate (diastereomer mixture) and 498 mg (3.61 mmol, 3 eq.) of potassium carbonate were added 12.2 ml of dioxane. Argon was passed through the reaction mixture for 5 min. 59 mg (72 μmol, 0.06 eq.) of [1,1-bis(diphenylphosphino)ferrocene]dichloropalladium dichloromethane complex were then added, and the mixture was stirred at 80° C. for 18 h. The reaction mixture was filtered through kieselguhr and washed with dichloromethane and acetonitrile, and the filtrate was concentrated. The crude product was purified by normal phase chromatography (eluent: cyclohexane/ethyl acetate, 50-100%). Yield: 474 mg (75% of theory).

LC/MS [Method 1]: Rₜ=1.02 min; MS (ESIpos): m/z=512 (M+H)⁺,

### Example 5.13B

### 2-{4-[5-Chloro-2-(2-fluoroethoxy)phenyl]-5-methoxy-2-oxopyridin-1(2H)-yl}-3-(1,4-dioxan-2-yl)propanoic acid (Diastereomer Mixture)

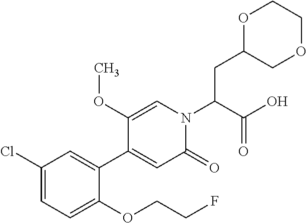 [SMILES: COc1cn(C(CC2COCCO2)C(=O)O)c(=O)cc1-c1cc(Cl)ccc1OCCF]

473 mg (0.901 mmol) of tert-butyl 2-{4-[5-chloro-2-(2-fluoroethoxy)phenyl]-5-methoxy-2-oxopyridin-1(2H)-yl}-3-(1,4-dioxan-2-yl)propanoate (diastereomer mixture) in 10 ml of hydrogen chloride in dioxane (4 M) were reacted according to General Method 6D. Yield: 445 mg (92% purity, 100% of theory)

LC/MS [Method 10]: Rₜ=1.41 min; MS (ESIpos): m/z=456 (M+H)⁺.

### Example 5.13C

### Methyl 4-{[2-{4-[5-chloro-2-(2-fluoroethoxy)phenyl]-5-methoxy-2-oxopyridin-1(2H)-yl}-3-(1,4-dioxan-2-yl)propanoyl]amino}benzoate (Diastereomer Mixture)

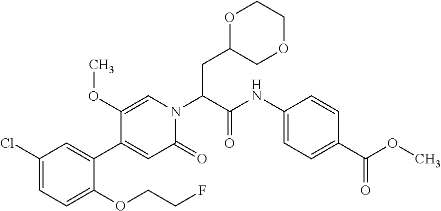 [SMILES: COC(=O)c1ccc(NC(=O)C(CC2COCCO2)n2cc(OC)c(-c3cc(Cl)ccc3OCCF)cc2=O)cc1]

70 mg (92% purity, 0.141 mmol) of 2-{4-[5-chloro-2-(2-fluoroethoxy)phenyl]-5-methoxy-2-oxopyridin-1(2H)-yl}-3-(1,4-dioxan-2-yl)propanoic acid (diastereomer mixture) and 32.7 mg (0.212 mmol) of methyl 4-aminobenzoate in 1 ml of pyridine were reacted according to General Method 5A. Yield: 71 mg (85% of theory).

LC/MS [Method 1]: Rₜ=0.99/1.01 min; MS (ESIpos): m/z=589/589 (M+H)⁺,

### Example 5.14A

### tert-Butyl 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoate (Diastereomer Mixture)

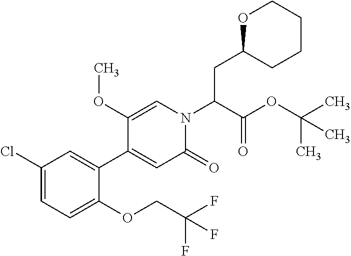 [SMILES: COc1cn(C(C[C@@H]2CCCCO2)C(=O)OC(C)(C)C)c(=O)cc1-c1cc(Cl)ccc1OCC(F)(F)F]

To 376 mg (1.3 mmol) of 2-bromo-4-chloro-1-(2,2,2-trifluoroethoxy)benzene, 899 mg (1.3 mmol, 67% purity, 1 eq.) of tert-butyl 2-[5-methoxy-2-oxo-4-(4,4,5,5-tetramethyl-1,3,2-dioxaborolan-2-yl)pyridin-1(2H)-yl]-3-[(2S)-tetrahydro-2H-pyran-2-yl]propanoate (diastereomer mixture) and 539 mg (3.90 mmol) of potassium carbonate were added 13.2 ml of dioxane. Argon was passed through the reaction mixture for 5 min. 63.7 mg (78 μmol, 0.06 eq.) of [1,1-bis(diphenylphosphino)ferrocene]dichloropalladium dichloromethane complex were then added, and the mixture was stirred at 80° C. for 4 h. The reaction mixture was filtered through kieselguhr and washed with dichloromethane and acetonitrile, and the filtrate was concentrated. The crude product was purified by normal phase chromatography (eluent: cyclohexane/ethyl acetate, 0-30%). Yield: 640 mg (80% purity, 72% of theory).

LC/MS [Method 10]: Rₜ=2.30 min; MS (ESIpos): m/z=546 (M+H)⁺.

### Example 5.14B

### 2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoic acid (Diastereomer Mixture)

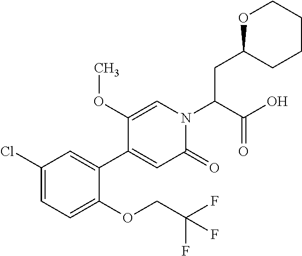 [SMILES: COc1cn(C(C[C@@H]2CCCCO2)C(=O)O)c(=O)cc1-c1cc(Cl)ccc1OCC(F)(F)F]

640 mg (0.938 mmol, 80% purity) of tert-butyl 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoate (diastereomer mixture) in 34 ml of hydrogen chloride in dioxane (4 M) were reacted according to General Method 6D. Yield: 530 mg (85% purity, 98% of theory)

LC/MS [Method 10]: Rₜ=1.79/1.81 min; MS (ESIpos): m/z=490/490 (M+H)⁺.

### Example 5.14C

### Methyl 4-[(2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]benzoate (Diastereomer Mixture)

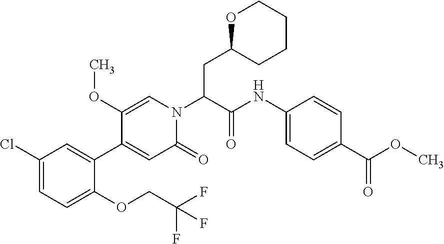 [SMILES: COC(=O)c1ccc(NC(=O)C(C[C@@H]2CCCCO2)n2cc(OC)c(-c3cc(Cl)ccc3OCC(F)(F)F)cc2=O)cc1]

130 mg (85% purity, 226 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 51.1 mg (338 μmol) of methyl 4-aminobenzoate in 2 ml of pyridine were reacted according to General Method 5A. Yield: 135 mg (96% of theory).

LC/MS [Method 2]: Rₜ=3.86/3.93 min; MS (ESIpos): m/z=623/623 (M+H)⁺.

### Example 5.15A

### tert-Butyl 2-{4-[5-chloro-2-(2,2,2-trifluoroethoxy)phenyl]-5-methoxy-2-oxopyridin-1(2H)-yl}-4-[(1-methylcyclobutyl)oxy]butanoate (Racemate)

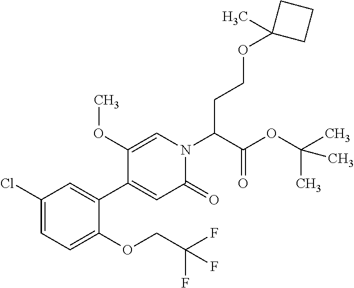 [SMILES: COc1cn(C(CCOC2(C)CCC2)C(=O)OC(C)(C)C)c(=O)cc1-c1cc(Cl)ccc1OCC(F)(F)F]

To 0.77 g (2.67 mmol) of 2-bromo-4-chloro-1-(2,2,2-trifluoroethoxy)benzene, 2.30 g (56% purity, 2.67 mmol) of tert-butyl 2-[5-methoxy-2-oxo-4-(4,4,5,5-tetramethyl-1,3,2-dioxaborolan-2-yl)pyridin-1(2H)-yl]-4-[(1-methylcyclobutyl)oxy]butanoate (racemate) and 1.11 g (8.02 mmol) of potassium carbonate were added 30.0 ml of dioxane. Argon was passed through the reaction mixture for 10 min. 65.5 mg (0.08 mmol) of [1,1-bis(diphenylphosphino)ferrocene]dichloropalladium/dichloromethane complex were then added, and the mixture was stirred at 80° C. for 2.5 h and at RT overnight. The reaction mixture was filtered through kieselguhr and washed through with dichloromethane, and the filtrate was concentrated. The crude product was purified by normal phase chromatography (eluent: cyclohexane/ethyl acetate gradient). Yield: 1.11 g (74% of theory).

LC/MS [Method 1]: Rₜ=1.27 min; MS (ESIpos): m/z=560 (M+H)⁺.

¹H-NMR (400 MHz, DMSO-d₆): δ [ppm]=7.49 (dd, 1H), 7.29 (d, 1H), 7.26-7.18 (m, 2H), 6.28 (s, 1H), 5.15-5.09 (m, 1H), 4.77-4.68 (m, 2H), 3.56 (s, 3H), 3.34-3.25 (m, partly concealed), 3.10-3.03 (m, 1H), 2.40-2.21 (m, 2H), 2.08-1.90 (m, 2H), 1.76-1.46 (m, 4H), 1.40 (s, 9H).

### Example 5.15B

### 2-{4-[5-Chloro-2-(2,2,2-trifluoroethoxy)phenyl]-5-methoxy-2-oxopyridin-1(2H)-yl}-4-[(1-methylcyclobutyl)oxy]butanoic acid (Racemate)

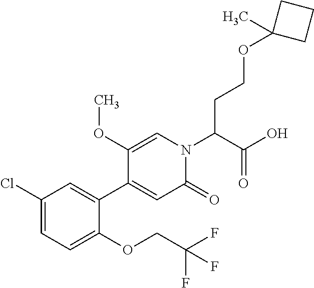 [SMILES: COc1cn(C(CCOC2(C)CCC2)C(=O)O)c(=O)cc1-c1cc(Cl)ccc1OCC(F)(F)F]

1.11 g (1.98 mmol) of tert-butyl 2-{4-[5-chloro-2-(2,2,2-trifluoroethoxy)phenyl]-5-methoxy-2-oxopyridin-1(2H)-yl}-4-[(1-methylcyclobutyl)oxy]butanoate (racemate) were initially charged in 18 ml of THF/ethanol (2:1), 9.92 ml of an aqueous lithium hydroxide solution (1N, 5.0 eq.) were added and the mixture was stirred at RT for 4 h. The mixture was then acidified to pH 4 with 1 N hydrochloric acid and extracted three times with 20 ml each time of ethyl acetate. The collected organic phases were washed once with saturated aqueous sodium chloride solution, dried over magnesium sulfate, filtered and concentrated. Yield: 1.01 g (quantitative).

LC/MS [Method 1]: Rₜ=1.03 min; MS (ESIpos): m/z=504 (M+H)⁺.

¹H-NMR (400 MHz, DMSO-d₆): δ [ppm]=12.91 (br. s., 1H), 7.52-7.46 (m, 1H), 7.31 (d, 1H), 7.28-7.22 (m, 2H), 6.28 (s, 1H), 5.21-5.14 (m, 1H), 4.79-4.69 (m, 2H), 3.56 (s, 3H), 3.35-3.24 (m, 1H), 3.07-2.99 (m, 1H), 2.46-2.35 (m, 1H), 2.34-2.23 (m, 1H), 2.07-1.88 (m, 2H), 1.75-1.46 (m, 4H), 1.20 (s, 3H).

### Example 5.16A

### tert-Butyl 2-{4-[5-chloro-2-(2,2,2-trifluoroethoxy)phenyl]-5-methoxy-2-oxopyridin-1(2H)-yl}pentanoate (Racemate)

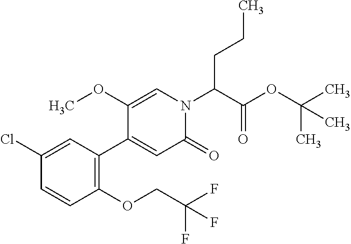 [SMILES: CCCC(C(=O)OC(C)(C)C)n1cc(OC)c(-c2cc(Cl)ccc2OCC(F)(F)F)cc1=O]

To 754 mg (57% purity, 1.05 mmol) of tert-butyl 2-[5-methoxy-2-oxo-4-(4,4,5,5-tetramethyl-1,3,2-dioxaborolan-2-yl)pyridin-1(2H)-yl]pentanoate (racemate) and 305 mg (1.05 mmol) of 2-bromo-4-chlorophenyl 2,2,2-trifluoroethyl ether were added 11 ml of dioxane. Argon was passed through the reaction mixture for 5 min. 86.1 mg (105 μmol) of [1,1-bis(diphenylphosphino)ferrocene]dichloropalladium-dichloromethane complex and 1.6 ml of an aqueous sodium carbonate solution (2.0 M, 3.2 mmol) were then added, and the mixture was stirred at 100° C. for 2 hours. The reaction mixture was filtered through kieselguhr and washed with dichloromethane and acetonitrile, and the filtrate was concentrated. The crude product was purified by normal phase chromatography (eluent: cyclohexane/ethyl acetate, 20-35%). Yield: 329 mg (62% of theory)

LC-MS (Method 10): Rₜ=2.26 min; MS (ESIpos): m/z=490 [M+H]⁺

### Example 5.16B

### 2-{4-[5-Chloro-2-(2,2,2-trifluoroethoxy)phenyl]-5-methoxy-2-oxopyridin-1(2H)-yl}pentanoic acid (Racemate)

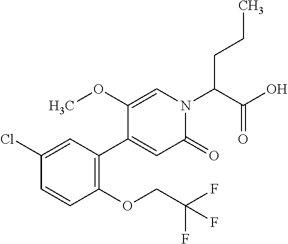 [SMILES: CCCC(C(=O)O)n1cc(OC)c(-c2cc(Cl)ccc2OCC(F)(F)F)cc1=O]

329 mg (651 μmol) of tert-butyl 2-{4-[5-chloro-2-(2,2,2-trifluoroethoxy)phenyl]-5-methoxy-2-oxopyridin-1(2H)-yl}pentanoate (racemate) were reacted in 6.5 ml of a solution of hydrogen chloride in dioxane (4M) according to General Method 6D. Yield: 279 mg (96% of theory).

LC-MS (Method 10): Rₜ=1.76 min; MS (ESIpos): m/z=434 [M+H]⁺

### Example 5.17A

### Ethyl 6-aminoimidazo [1,2-a]pyridine-2-carboxylate

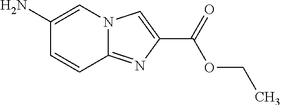

A solution of 250 mg (1.01 mmol) of ethyl 6-nitroimidazo[1,2-a]pyridine-2-carboxylate in 20 ml of ethanol was hydrogenated in the presence of 30 mg of palladium (10% on activated carbon) at RT and standard pressure for 5 h. The reaction mixture was then filtered through Celite and the residue was washed with ethanol. The combined filtrates were concentrated under reduced pressure and dried. Yield: 215 mg (quant.).

LC/MS (Method 5): Rₜ=1.40 min; MS (ESIpos): m/z=206 (M+H)⁺,

¹H-NMR (400 MHz, DMSO-d₆): δ [ppm]=8.33 (s, 1H), 7.66 (s, 1H), 7.37 (d, 1H), 6.94 (dd, 1H), 5.11 (s, 2H), 4.26 (q, 2H), 1.29 (t, 3H).

### Example 5.17B

### Ethyl 6-1 {[4-tert-butoxy-2-{4-[5-chloro-2-(2,2,2-trifluoroethoxy)phenyl]-5-methoxy-2-oxopyridin-1(2H)-yl}butanoyl]amino}imidazo[1,2-a]pyridine-2-carboxylate (Racemate)

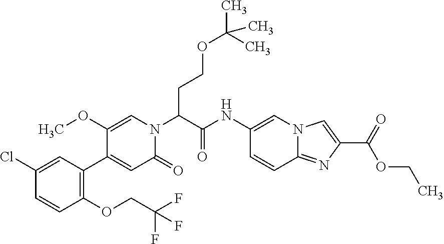 [SMILES: CCOC(=O)c1cn2cc(NC(=O)C(CCOC(C)(C)C)n3cc(OC)c(-c4cc(Cl)ccc4OCC(F)(F)F)cc3=O)ccc2n1]

95 mg (193 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 60.1 mg (290 μmol, 1.5 eq.) of ethyl 6-aminoimidazo[1,2-a]pyridine-2-carboxylate were reacted according to General Method 5A. Yield: 116 mg (88% of theory).

LC-MS (Method 1): Rₜ=1.12 min; MS (ESIpos): m/z=679 [M+H]⁺

### Example 5.17C

### Methyl 5-({2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)thiophene-2-carboxylate (Diastereomer Mixture)

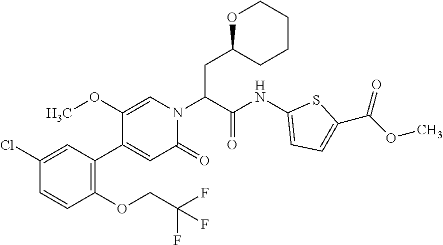 [SMILES: COC(=O)c1ccc(NC(=O)C(C[C@@H]2CCCCO2)n2cc(OC)c(-c3cc(Cl)ccc3OCC(F)(F)F)cc2=O)s1]

55.0 mg (80% purity, 89.8 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 21.2 mg (0.135 mmol) of methyl 5-aminothiophene-2-carboxylate in 1.0 ml of pyridine were reacted according to General Method 5A. Yield: 55.2 mg (98% of theory)

LC-MS (Method 10): Rₜ=2.13/2.16 min; MS (ESIpos): m/z=629/629 [M+H]⁺

### Example 5.17D

### Ethyl 6-({2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)imidazo[1,2-a]pyridine-2-carboxylate (Diastereomer Mixture)

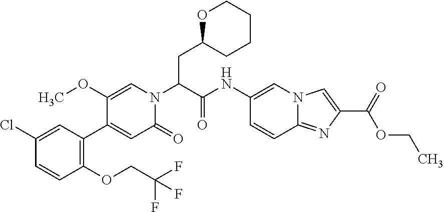 [SMILES: CCOC(=O)c1cn2cc(NC(=O)C(C[C@@H]3CCCCO3)n3cc(OC)c(-c4cc(Cl)ccc4OCC(F)(F)F)cc3=O)ccc2n1]

60.0 mg (85% purity, 104 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 34.2 mg (0.156 mmol) of ethyl 6-aminoimidazo[1,2-a]pyridine-2-carboxylate in 0.92 ml of pyridine were reacted according to General Method 5A. Yield: 60 mg (85% of theory).

LC-MS (Method 1): Rₜ=1.07 min; MS (ESIpos): m/z=677 [M+H]⁺

### Working Examples

### General Method 1: Hydrolysis of a Tert-Butyl Ester or a Boc-Protected Amine Using TFA

At 0° C. to RT, TFA (10-20 eq.) was added to a solution of the appropriate tert-butyl ester derivative or a Boc-protected amine (1.0 eq.) in dichloromethane (about 25 ml/mmol), and the mixture was stirred at RT for 1 to 8 h. Subsequently, the reaction mixture was concentrated under reduced pressure. The residue was co-evaporated repeatedly with dichloromethane and/or toluene. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

General Method 2: Hydrolysis of a Methyl or Ethyl Ester with Lithium Hydroxide

At RT, lithium hydroxide (2-4 eq.) was added to a solution of the appropriate ester (1.0 eq.) in a mixture of tetrahydrofuran/water (3:1, about 7-15 ml/mmol), and the mixture was stirred at RT. The reaction mixture was then adjusted to pH 1 using aqueous hydrochloric acid solution (1N). After addition of water/ethyl acetate, the aqueous phase was extracted three times with ethyl acetate. The combined organic phases were dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 3: Amide Coupling Using HATU/DIEA

Under argon and at RT, the appropriate amine (1.1-1.2 eq.), N,N-diisopropylethylamine (DIEA) (2.2-3.0 eq.) and a solution of HATU (1.2 eq.) in a little dimethylformamide were added to a solution of the appropriate carboxylic acid (1.0 eq.) in dimethylformamide (about 7-70 ml/mmol). The reaction mixture was stirred at RT. After addition of water/ethyl acetate and phase separation, the organic phase was washed with water and with saturated aqueous sodium chloride solution, dried (sodium sulfate or magnesium sulfate), filtered and concentrated under reduced pressure. The crude product was then purified either by means of normal phase chromatography (cyclohexane/ethyl acetate mixtures or dichloromethane/methanol mixtures) or preparative RP-HPLC (water/acetonitrile gradient or water/methanol gradient).

### General Method 4: Amide Coupling Using T3P/DIEA

Under argon and at 0° C. or RT, N,N-diisopropylethylamine (3 eq.) and propylphosphonic anhydride (T3P, 50% in dimethylformamide or in ethyl acetate, 3 eq.) were added dropwise to a solution of the carboxylic acid and the appropriate amine (1.1-1.5 eq.) in dimethylformamide (0.15-0.05 mmol). The reaction mixture was stirred at RT and then concentrated under reduced pressure. After addition of water/ethyl acetate and phase separation, the aqueous phase was extracted twice with ethyl acetate. The combined organic phases were dried (sodium sulfate or magnesium sulfate), filtered and concentrated under reduced pressure. The crude product was then purified either by flash chromatography (cyclohexane/ethyl acetate mixtures or dichloromethane/methanol mixtures) or preparative HPLC (Reprosil C18, water/acetonitrile gradient or water/methanol gradient).

### General Method 5: Amide Coupling Using T3P/Pyridine

A solution of the appropriate carboxylic acid (1 eq.) and the appropriate amine (1.0-2.5 eq.) in pyridine (about 0.1M) was heated to 60 to 90° C., and T3P (50% in dimethylformamide or in ethyl acetate, 1.5 to 4 eq.) was added dropwise. Alternatively, T3P (50% in dimethylformamide or in ethyl acetate, 1.5 to 4 eq.) was added at RT and the mixture was then stirred at RT or heated to RT to 90° C. After 1 to 20 h, the reaction mixture was cooled to RT, and water and ethyl acetate were added. The aqueous phase was extracted with ethyl acetate. The combined organic phases were washed with aqueous buffer solution (pH=5), with saturated aqueous sodium hydrogencarbonate solution and with saturated aqueous sodium chloride solution, dried over sodium sulfate and concentrated under reduced pressure. The crude product was then optionally purified either by normal phase chromatography (cyclohexane/ethyl acetate mixtures or dichloromethane/methanol mixtures) or preparative RP-HPLC (water/acetonitrile gradient or water/methanol gradient). Alternatively, the reaction solution can also, without further workup, be diluted with acetonitrile and separated by means of preparative RP-HPLC.

### Example 1

### 4-[(2-{4-[5-Chloro-2-(2,2,2-trifluoroethoxy)phenyl]-5-methoxy-2-oxopyridin-1(2H)-yl}-4-methoxybutanoyl)amino]benzoic acid (Racemate)

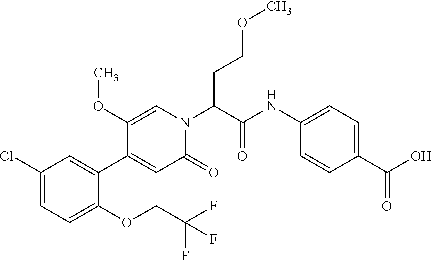 [SMILES: COCCC(C(=O)Nc1ccc(C(=O)O)cc1)n1cc(OC)c(-c2cc(Cl)ccc2OCC(F)(F)F)cc1=O]

123 mg (0.211 mmol) of methyl 4-[(2-{4-[5-chloro-2-(2,2,2-trifluoroethoxy)phenyl]-5-methoxy-2-oxopyridin-1(2H)-yl}-4-methoxybutanoyl)amino]benzoate (racemate) were reacted according to General Method 2. Yield: 90 mg (75% of theory)

LC/MS [Method 10]: Rₜ=1.75 min; MS (ESIpos): m/z=569 (M+H)⁺,

¹H-NMR (400 MHz, DMSO-d₆): δ [ppm]=12.74 (br. s., 1H), 10.70 (s, 1H), 7.93-7.87 (m, 2H), 7.78-7.72 (m, 2H), 7.49 (dd, 1H), 7.37-7.32 (m, 2H), 7.26 (d, 1H), 6.32 (s, 1H), 5.74 (dd, 1H), 4.81-4.70 (m, 2H), 3.60 (s, 3H), 3.40-3.33 (m, 1H), 3.27-3.20 (m, 1H), 3.19 (s, 3H), 2.44-2.35 (m, 2H).

### Example 2

### 4-[(2-{4-[5-Chloro-2-(2,2,2-trifluoroethoxy)phenyl]-5-methoxy-2-oxopyridin-1(2H)-yl}-4-methoxybutanoyl)amino]benzoic acid (enantiomer 2)

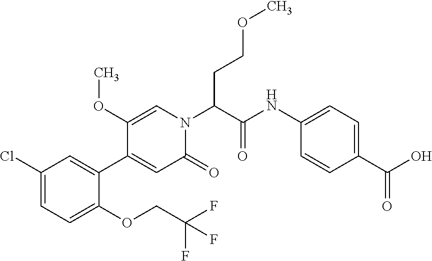 [SMILES: COCCC(C(=O)Nc1ccc(C(=O)O)cc1)n1cc(OC)c(-c2cc(Cl)ccc2OCC(F)(F)F)cc1=O]

Enantiomer separation of 90 mg of 4-[(2-{4-[5-chloro-2-(2,2,2-trifluoroethoxy)phenyl]-5-methoxy-2-oxopyridin-1(2H)-yl}-4-methoxybutanoyl)amino]benzoic acid (racemate) gave 39 mg of enantiomer 1 (chiral HPLC: Rₜ=3.1 min) and 40 mg of the Example 2 title compound (enantiomer 2): chiral HPLC: Rₜ=7.6 min; 100% ee.

Separation method: column: Chiralpak AZ-H 5 μm 250 mm×20 mm; eluent: 75% carbon dioxide/55% ethanol; temperature: 40° C.; flow rate: 80 ml/min; pressure: 100 bar; UV detection: 210 nm.

Analysis: column: Daicel AD 5 μm 250 mm×4.6 mm; eluent: 80% carbon dioxide, 20% ethanol; flow rate: 3 ml/min; UV detection: 210 nm.

### Example 3

### 4-[(2-{4-[5-Chloro-2-(2,2,2-trifluoroethoxy)phenyl]-5-methoxy-2-oxopyridin-1(2H)-yl}-4-methoxybutanoyl)amino]-2-fluorobenzamide (Racemate)

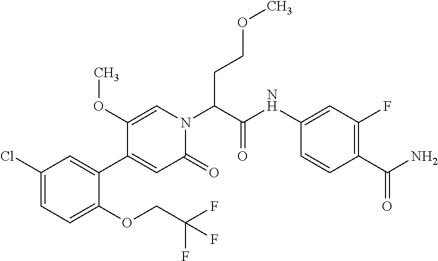 [SMILES: COCCC(C(=O)Nc1ccc(C(N)=O)c(F)c1)n1cc(OC)c(-c2cc(Cl)ccc2OCC(F)(F)F)cc1=O]

60 mg (0.10 mmol, 75% purity) of 2-{4-[5-chloro-2-(2,2,2-trifluoroethoxy)phenyl]-5-methoxy-2-oxopyridin-1(2H)-yl}-4-methoxybutanoic acid (racemate) and 23.8 mg (0.15 mmol, 1.5 eq.) of 4-amino-2-fluorobenzamide were reacted according to General Method 5. Yield: 37 mg (63% of theory)

LC/MS [Method 10]: Rₜ=1.70 min; MS (ESIpos): m/z=586 (M+H)⁺,

¹H-NMR (400 MHz, DMSO-d₆): δ [ppm]=10.76 (s, 1H), 7.72-7.62 (m, 2H), 7.57-7.47 (m, 3H), 7.43 (dd, 1H), 7.36-7.31 (m, 2H), 7.26 (d, 1H), 6.33 (s, 1H), 5.74-5.66 (m, 1H), 4.81-4.71 (m, 2H), 3.60 (s, 3H), 3.40-3.33 (m, 1H), 3.27-3.19 (m, 1H), 3.19 (s, 3H), 2.44-2.34 (m, 2H).

### Example 4

### 5-[(2-{4-[5-Chloro-2-(2,2,2-trifluoroethoxy)phenyl]-5-methoxy-2-oxopyridin-1(2H)-yl}-4-methoxybutanoyl)amino]-N-methylpyridine-2-carboxamide (Racemate)

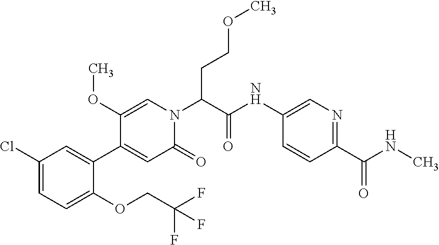 [SMILES: CNC(=O)c1ccc(NC(=O)C(CCOC)n2cc(OC)c(-c3cc(Cl)ccc3OCC(F)(F)F)cc2=O)cn1]

60 mg (0.10 mmol, 75% purity) of 2-{4-[5-chloro-2-(2,2,2-trifluoroethoxy)phenyl]-5-methoxy-2-oxopyridin-1(2H)-yl}-4-methoxybutanoic acid (racemate) and 23.1 mg (0.15 mmol, 1.5 eq.) of 5-amino-N-methylpyridine-2-carboxamide were reacted according to General Method 5. Yield: 40 mg (69% of theory)

LC/MS [Method 10]: Rₜ=1.71 min; MS (ESIpos): m/z=583 (M+H)⁺,

¹H-NMR (400 MHz, DMSO-d₆): δ [ppm]=10.83 (s, 1H), 8.87 (d, 1H), 8.65 (q, 1H), 8.21 (dd, 1H), 8.00 (d, 1H), 7.50 (dd, 1H), 7.36-7.32 (m, 2H), 7.26 (d, 1H), 6.33 (s, 1H), 5.71 (dd, 1H), 4.81-4.71 (m, 2H), 3.60 (s, 3H), 3.43-3.34 (m, 1H), 3.27-3.20 (m, 1H), 3.19 (s, 3H), 2.80 (d, 3H), 2.46-2.36 (m, 2H).

### Example 5

### 2-{4-[5-Chloro-2-(2,2,2-trifluoroethoxy)phenyl]-5-methoxy-2-oxopyridin-1(2H)-yl}-4-methoxy-N-(2-methyl-2H-indazol-5-yl)butanamide (Racemate)

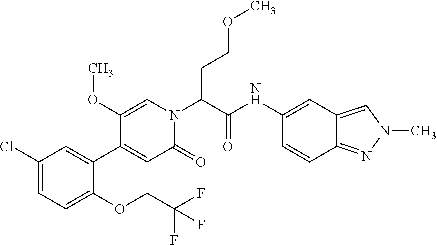 [SMILES: COCCC(C(=O)Nc1ccc2nn(C)cc2c1)n1cc(OC)c(-c2cc(Cl)ccc2OCC(F)(F)F)cc1=O]

60 mg (0.10 mmol, 75% purity) of 2-{4-[5-chloro-2-(2,2,2-trifluoroethoxy)phenyl]-5-methoxy-2-oxopyridin-1(2H)-yl}-4-methoxybutanoic acid (racemate) and 24.8 mg (0.15 mmol, 89% purity, 1.5 eq.) of 2-methyl-2H-indazole-5-amine were reacted according to General Method 2. Yield: 30 mg (51% of theory)

LC/MS [Method 10]: Rₜ=1.74 min; MS (ESIpos): m/z=579 (M+H)⁺,

¹H-NMR (400 MHz, DMSO-d₆): δ [ppm]=10.35 (s, 1H), 8.25 (s, 1H), 8.12 (d, 1H), 7.54 (d, 1H), 7.49 (dd, 1H), 7.39 (s, 1H), 7.34 (d, 1H), 7.31 (dd, 1H), 7.26 (d, 1H), 6.33 (s, 1H), 5.77 (dd, 1H), 4.81-4.71 (m, 2H), 4.13 (s, 3H), 3.60 (s, 3H), 3.40-3.33 (m, 1H), 3.28-3.22 (m, 1H), 3.20 (s, 3H), 2.44-2.29 (m, 2H).

### Example 6

### 4-[(2-{4-[5-Chloro-2-(2,2,2-trifluoroethoxy)phenyl]-5-methoxy-2-oxopyridin-1(2H)-yl}-4-methoxybutanoyl)amino]-2-fluoro-N-methylbenzamide (Racemate)

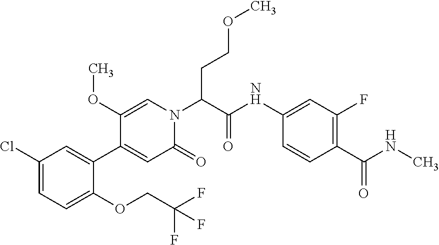 [SMILES: CNC(=O)c1ccc(NC(=O)C(CCOC)n2cc(OC)c(-c3cc(Cl)ccc3OCC(F)(F)F)cc2=O)cc1F]

60 mg (0.10 mmol, 75% purity) of 2-{4-[5-chloro-2-(2,2,2-trifluoroethoxy)phenyl]-5-methoxy-2-oxopyridin-1(2H)-yl}-4-methoxybutanoic acid (racemate) and 25.2 mg (0.15 mmol, 1.5 eq.) of 4-amino-2-fluoro-N-methylbenzamide were reacted according to General Method 5. Yield: 40 mg (67% of theory)

LC/MS [Method 10]: Rₜ=1.78 min; MS (ESIpos): m/z=600 (M+H)⁺,

¹H-NMR (400 MHz, DMSO-d₆): δ [ppm]=10.75 (s, 1H), 8.12-8.02 (m, 1H), 7.69-7.60 (m, 2H), 7.50 (dd, 1H), 7.42 (dd, 1H), 7.35-7.31 (m, 2H), 7.26 (d, 1H), 6.33 (s, 1H), 5.73-5.66 (m, 1H), 4.81-4.70 (m, 2H), 3.60 (s, 3H), 3.40-3.33 (m, 1H), 3.27-3.20 (m, 1H), 3.19 (s, 3H), 2.76 (d, 3H), 2.44-2.34 (m, 2H).

### Example 7

### 4-[(4-tert-Butoxy-2-{4-[5-chloro-2-(2,2,2-trifluoroethoxy)phenyl]-5-methoxy-2-oxopyridin-1(2H)-yl}butanoyl)amino]benzoic acid (Racemate)

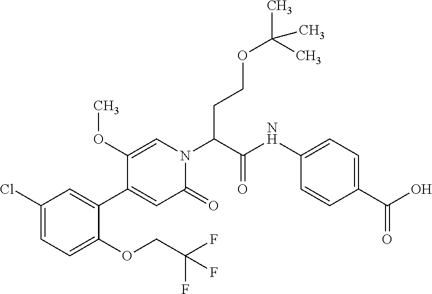 [SMILES: COc1cn(C(CCOC(C)(C)C)C(=O)Nc2ccc(C(=O)O)cc2)c(=O)cc1-c1cc(Cl)ccc1OCC(F)(F)F]

165 mg (0.264 mmol) of methyl 4-[(4-tert-butoxy-2-{4-[5-chloro-2-(2,2,2-trifluoroethoxy)phenyl]-5-methoxy-2-oxopyridin-1(2H)-yl}butanoyl)amino]benzoate (racemate) were reacted according to General Method 2. Yield: 121 mg (75% of theory)

LC/MS [Method 1]: Rₜ=1.07 min; MS (ESIpos): m/z=611 (M+H)⁺,

¹H-NMR (400 MHz, DMSO-d₆): δ [ppm]=12.73 (br. s., 1H), 10.70 (s, 1H), 7.93-7.87 (m, 2H), 7.80-7.74 (m, 2H), 7.49 (dd, 1H), 7.32 (s, 1H), 7.30 (d, 1H), 7.26 (d, 1H), 6.32 (s, 1H), 5.80-5.73 (m, 1H), 4.84-4.66 (m, 2H), 3.60 (s, 3H), 3.41-3.34 (m, 1H), 3.28-3.20 (m, 1H), 2.40-2.29 (m, 2H), 1.03 (s, 9H).

### Example 8

### 4-[(4-tert-Butoxy-2-{4-[5-chloro-2-(2,2,2-trifluoroethoxy)phenyl]-5-methoxy-2-oxopyridin-1(2H)-yl}butanoyl)amino]benzoic acid (enantiomer 2)

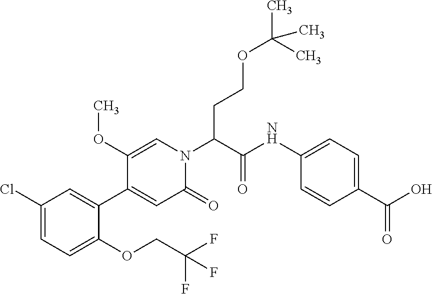 [SMILES: COc1cn(C(CCOC(C)(C)C)C(=O)Nc2ccc(C(=O)O)cc2)c(=O)cc1-c1cc(Cl)ccc1OCC(F)(F)F]

Enantiomer separation of 118 mg of 4-[(4-tert-butoxy-2-{4-[5-chloro-2-(2,2,2-trifluoroethoxy)phenyl]-5-methoxy-2-oxopyridin-1(2H)-yl}butanoyl)amino]benzoic acid (racemate) gave 50 mg of enantiomer 1 (chiral HPLC: Rₜ=2.6 min) and 43 mg of the Example 8 title compound (enantiomer 2): chiral HPLC: Rₜ=6.0 min; 100% ee.

Separation method: column: Daicel Chiralpak AZ-H 5 μm, 250 mm×20 mm; eluent: 80% carbon dioxide/20% ethanol; temperature: 40° C.; flow rate: 80 ml/min; pressure: 90 bar; UV detection: 210 nm.

Analysis: column: Daicel AZ 5 μm, 250 mm×4.6 mm; eluent: 70% carbon dioxide, 30% ethanol; flow rate: 3 ml/min; UV detection: 210 nm.

### Example 9

### 4-tert-Butoxy-2-{4-[5-chloro-2-(2,2,2-trifluoroethoxy)phenyl]-5-methoxy-2-oxopyridin-1(2H)-yl}-N-(2-methyl-2H-indazol-5-yl)butanamide (Racemate)

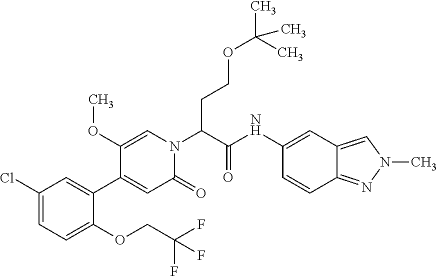 [SMILES: COc1cn(C(CCOC(C)(C)C)C(=O)Nc2ccc3nn(C)cc3c2)c(=O)cc1-c1cc(Cl)ccc1OCC(F)(F)F]

35 mg (69 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 15.2 mg (104 μmol, 1.5 eq.) of 2-methyl-2H-indazole-5-amine were reacted according to General Method 5. Yield: 24 mg (56% of theory)

LC/MS [Method 10]: Rₜ=2.02 min; MS (ESIpos): m/z=621 (M+H)⁺,

¹H-NMR (400 MHz, DMSO-d₆): δ [ppm]=10.33 (s, 1H), 8.24 (s, 1H), 8.13 (d, 1H), 7.54 (d, 1H), 7.49 (dd, 1H), 7.36 (s, 1H), 7.34-7.28 (m, 2H), 7.26 (d, 1H), 6.32 (s, 1H), 5.83-5.74 (m, 1H), 4.84-4.66 (m, 2H), 4.13 (s, 3H), 3.60 (s, 3H), 3.41-3.33 (m, 1H), 3.28-3.22 (m, 1H), 2.39-2.29 (m, 2H), 1.04 (s, 9H).

### Example 10

### 4-[(4-tert-Butoxy-2-{4-[5-chloro-2-(2,2,2-trifluoroethoxy)phenyl]-5-methoxy-2-oxopyridin-1(2H)-yl}butanoyl)amino]-2-fluorobenzamide (Racemate)

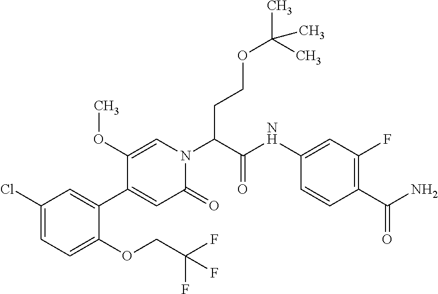 [SMILES: COc1cn(C(CCOC(C)(C)C)C(=O)Nc2ccc(C(N)=O)c(F)c2)c(=O)cc1-c1cc(Cl)ccc1OCC(F)(F)F]

35 mg (69 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 16.4 mg (104 μmol, 1.5 eq.) of 4-amino-2-fluorobenzamide were reacted according to General Method 5. Yield: 34 mg (78% of theory)

LC/MS [Method 10]: Rₜ=1.98 min; MS (ESIpos): m/z=628 (M+H)⁺,

¹H-NMR (400 MHz, DMSO-d₆): δ [ppm]=10.76 (s, 1H), 7.73-7.61 (m, 2H), 7.58-7.40 (m, 4H), 7.33-7.28 (m, 2H), 7.26 (d, 1H), 6.32 (s, 1H), 5.74 (dd, 1H), 4.85-4.65 (m, 2H), 3.60 (s, 3H), 3.41-3.34 (m, 1H), 3.28-3.20 (m, 1H), 2.41-2.29 (m, 2H), 1.03 (s, 9H).

### Example 11

### 5-[(4-tert-Butoxy-2-{4-[5-chloro-2-(2,2,2-trifluoroethoxy)phenyl]-5-methoxy-2-oxopyridin-1(2H)-yl}butanoyl)amino]pyridine-2-carboxamide (Racemate)

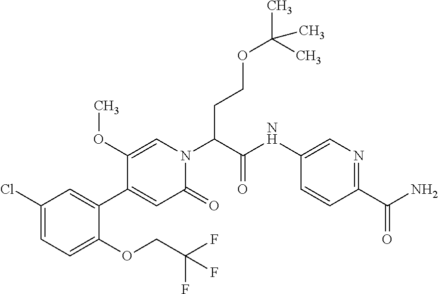 [SMILES: COc1cn(C(CCOC(C)(C)C)C(=O)Nc2ccc(C(N)=O)nc2)c(=O)cc1-c1cc(Cl)ccc1OCC(F)(F)F]

35 mg (69 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 14.6 mg (104 μmol, 1.5 eq.) of 5-aminopyridine-2-carboxamide were reacted according to General Method 5. Yield: 32 mg (76% of theory)

LC/MS [Method 10]: Rₜ=1.92 min; MS (ESIpos): m/z=611 (M+H)⁺,

¹H-NMR (400 MHz, DMSO-d₆): δ [ppm]=10.85 (s, 1H), 8.90-8.83 (m, 1H), 8.24 (dd, 1H), 8.04-7.97 (m, 2H), 7.55-7.45 (m, 2H), 7.32 (s, 1H), 7.30 (d, 1H), 7.28-7.21 (m, 1H), 6.33 (s, 1H), 5.80-5.72 (m, 1H), 4.84-4.66 (m, 2H), 3.61 (s, 3H), 3.43-3.35 (m, 1H), 3.27-3.20 (m, 1H), 2.44-2.34 (m, 2H), 1.03 (s, 9H).

### Example 12

### 5-[(4-tert-Butoxy-2-{4-[5-chloro-2-(2,2,2-trifluoroethoxy)phenyl]-5-methoxy-2-oxopyridin-1(2H)-yl}butanoyl)amino]-N-methylpyridine-2-carboxamide (Racemate)

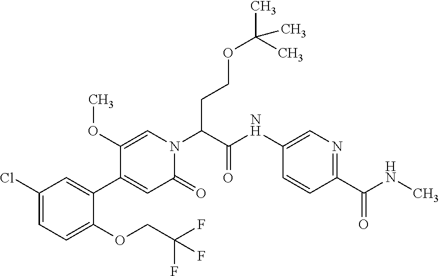 [SMILES: CNC(=O)c1ccc(NC(=O)C(CCOC(C)(C)C)n2cc(OC)c(-c3cc(Cl)ccc3OCC(F)(F)F)cc2=O)cn1]

35 mg (69 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 16 mg (104 μmol, 1.5 eq.) of 5-amino-N-methylpyridine-2-carboxamide were reacted according to General Method 5. Yield: 33 mg (77% of theory)

LC/MS [Method 10]: Rₜ=2.00 min; MS (ESIpos): m/z=625 (M+H)⁺,

¹H-NMR (400 MHz, DMSO-d₆): δ [ppm]=10.84 (s, 1H), 8.89 (d, 1H), 8.66 (q, 1H), 8.22 (dd, 1H), 8.00 (d, 1H), 7.49 (dd, 1H), 7.32 (s, 1H), 7.30 (d, 1H), 7.26 (d, 1H), 6.33 (s, 1H), 5.80-5.72 (m, 1H), 4.84-4.66 (m, 2H), 3.60 (s, 3H), 3.43-3.35 (m, 1H), 3.29-3.19 (m, 1H), 2.80 (d, 3H), 2.44-2.34 (m, 2H), 1.03 (s, 9H).

### Example 13

### 4-{[2-{4-[5-Chloro-2-(2,2,2-trifluoroethoxy)phenyl]-5-methoxy-2-oxopyridin-1(2H)-yl}-3-(1-methyl-1H-pyrazol-3-yl)propanoyl] amino}benzoic acid (Racemate)

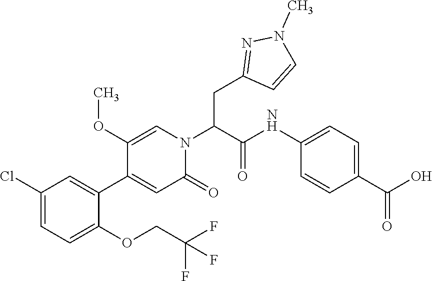 [SMILES: COc1cn(C(Cc2ccn(C)n2)C(=O)Nc2ccc(C(=O)O)cc2)c(=O)cc1-c1cc(Cl)ccc1OCC(F)(F)F]

33 mg (53 μmol) of methyl 4-{[2-{4-[5-chloro-2-(2,2,2-trifluoroethoxy)phenyl]-5-methoxy-2-oxopyridin-1(2H)-yl}-3-(1-methyl-1H-pyrazol-3-yl)propanoyl]amino}benzoate (racemate) were reacted according to General Method 2. Yield: 16 mg (50% of theory)

LC/MS [Method 10]: Rₜ=1.70 min; MS (ESIpos): m/z=605 (M+H)⁺,

¹H-NMR (400 MHz, DMSO-d₆): δ [ppm]=12.74 (br. s., 1H), 10.81 (s, 1H), 7.94-7.87 (m, 2H), 7.79-7.72 (m, 2H), 7.51-7.46 (m, 2H), 7.44 (s, 1H), 7.30 (d, 1H), 7.25 (d, 1H), 6.26 (s, 1H), 5.95-5.86 (m, 2H), 4.80-4.69 (m, 2H), 3.74 (s, 3H), 3.59 (s, 3H), 3.50-3.36 (m, 2H).

### Example 14

### 2-{4-[5-Chloro-2-(2,2,2-trifluoroethoxy)phenyl]-5-methoxy-2-oxopyridin-1(2H)-yl}-N-(2-methyl-2H-indazol-5-yl)-3-(1-methyl-1H-pyrazol-3-yl)propanamide (Racemate)

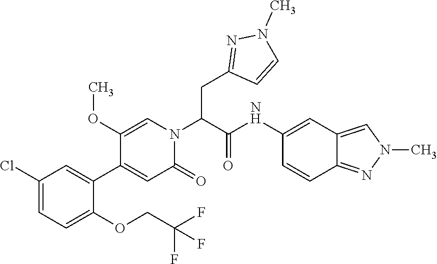 [SMILES: COc1cn(C(Cc2ccn(C)n2)C(=O)Nc2ccc3nn(C)cc3c2)c(=O)cc1-c1cc(Cl)ccc1OCC(F)(F)F]

40 mg (75 μmol) of 2-{4-[5-chloro-2-(2,2,2-trifluoroethoxy)phenyl]-5-methoxy-2-oxopyridin-1(2H)-yl}-3-(1-methyl-1H-pyrazol-3-yl)propanoic acid (racemate) and 18.6 mg (113 μmol, 89% purity, 1.5 eq.) of 2-methyl-2H-indazole-5-amine were reacted according to General Method 5. Yield: 22 mg (47% of theory)

LC/MS [Method 1]: Rₜ=0.96 min; MS (ESIpos): m/z=615 (M+H)⁺,

¹H-NMR (400 MHz, DMSO-d₆): δ [ppm]=10.44 (s, 1H), 8.25 (s, 1H), 8.13 (d, 1H), 7.55 (d, 1H), 7.51-7.45 (m, 3H), 7.33-7.27 (m, 2H), 7.25 (d, 1H), 6.26 (s, 1H), 5.96-5.89 (m, 2H), 4.80-4.69 (m, 2H), 4.13 (s, 3H), 3.74 (s, 3H), 3.60 (s, 3H), 3.47-3.35 (m, 2H).

### Example 15

### 5-{[2-{4-[5-Chloro-2-(2,2,2-trifluoroethoxy)phenyl]-5-methoxy-2-oxopyridin-1(2H)-yl}-3-(1-methyl-1H-pyrazol-3-yl)propanoyl]amino}-N-methylpyridine-2-carboxamide (Racemate)

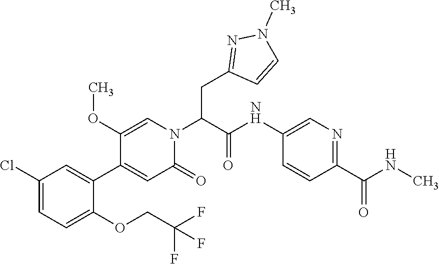 [SMILES: CNC(=O)c1ccc(NC(=O)C(Cc2ccn(C)n2)n2cc(OC)c(-c3cc(Cl)ccc3OCC(F)(F)F)cc2=O)cn1]

40 mg (75 μmol) of 2-{4-[5-chloro-2-(2,2,2-trifluoroethoxy)phenyl]-5-methoxy-2-oxopyridin-1(2H)-yl}-3-(1-methyl-1H-pyrazol-3-yl)-propanoic acid (racemate) and 17.5 mg (113 μmol, 1.5 eq.) of 5-amino-N-methylpyridine-2-carboxamide were reacted according to General Method 5. Yield: 38 mg (81% of theory)

LC/MS [Method 1]: Rₜ=0.95 min; MS (ESIpos): m/z=619 (M+H)⁺,

¹H-NMR (400 MHz, DMSO-d₆): δ [ppm]=10.95 (s, 1H), 8.87 (d, 1H), 8.70-8.62 (m, 1H), 8.20 (dd, 1H), 8.01 (d, 1H), 7.51-7.46 (m, 2H), 7.43 (s, 1H), 7.29 (d, 1H), 7.25 (d, 1H), 6.27 (s, 1H), 5.93 (d, 1H), 5.87 (dd, 1H), 4.80-4.69 (m, 2H), 3.74 (s, 3H), 3.59 (s, 3H), 3.53-3.40 (m, 2H), 2.80 (d, 3H).

### Example 16

### 4-{[2-{4-[5-Chloro-2-(2,2,2-trifluoroethoxy)phenyl]-5-methoxy-2-oxopyridin-1 (2H)-yl}-3-(1-methyl-1H-pyrazol-3-yl)propanoyl]amino}-2-fluorobenzamide (Racemate)

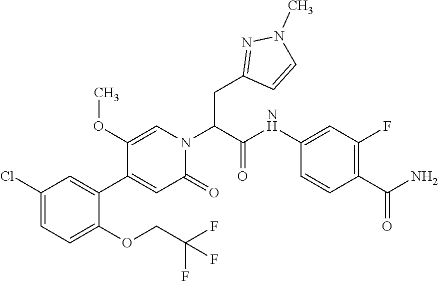 [SMILES: COc1cn(C(Cc2ccn(C)n2)C(=O)Nc2ccc(C(N)=O)c(F)c2)c(=O)cc1-c1cc(Cl)ccc1OCC(F)(F)F]

40 mg (75 μmol) of 2-{4-[5-chloro-2-(2,2,2-trifluoroethoxy)phenyl]-5-methoxy-2-oxopyridin-1(2H)-yl}-3-(1-methyl-1H-pyrazol-3-yl)propanoic acid (racemate) and 17.9 mg (113 μmol, 1.5 eq.) of 4-amino-2-fluorobenzamide were reacted according to General Method 5. Yield: 39 mg (83% of theory)

LC/MS [Method 1]: Rₜ=0.94 min; MS (ESIpos): m/z=622 (M+H)⁺,

¹H-NMR (400 MHz, DMSO-d₆): δ [ppm]=10.87 (s, 1H), 7.73-7.62 (m, 2H), 7.58-7.45 (m, 4H), 7.45-7.38 (m, 2H), 7.29 (d, 1H), 7.25 (d, 1H), 6.27 (s, 1H), 5.92 (d, 1H), 5.86 (dd, 1H), 4.82-4.67 (m, 2H), 3.74 (s, 3H), 3.59 (s, 3H), 3.51-3.36 (m, 2H).

### Example 17

### 4-{[2-{4-[5-Chloro-2-(2,2,2-trifluoroethoxy)phenyl]-5-methoxy-2-oxopyridin-1(2H)-yl}-3-(1,4-dioxan-2-yl)propanoyl]amino}benzoic acid (Diastereomer Mixture)

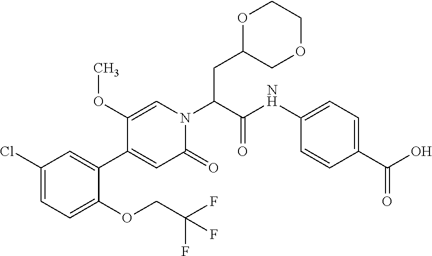 [SMILES: COc1cn(C(CC2COCCO2)C(=O)Nc2ccc(C(=O)O)cc2)c(=O)cc1-c1cc(Cl)ccc1OCC(F)(F)F]

35 mg (56 μmol) of methyl 4-{[2-{4-[5-chloro-2-(2,2,2-trifluoroethoxy)phenyl]-5-methoxy-2-oxopyridin-1(2H)-yl}-3-(1,4-dioxan-2-yl)propanoyl]amino}benzoate (diastereomer mixture) were reacted according to General Method 2. Yield: 29 mg (85% of theory)

LC/MS [Method 10]: Rₜ=1.73/1.76 min; MS (ESIpos): m/z=611/611 (M+H)⁺,

¹H-NMR (400 MHz, DMSO-d₆): δ [ppm]=12.75 (br. s., 1H), 10.71/10.68 (2×s, 1H), 7.94-7.85 (m, 2H), 7.79-7.70 (m, 2H), 7.52-7.46 (m, 1H), 7.39-7.30 (m, 2H), 7.30-7.24 (m, 1H), 6.34/6.32 (2×s, 1H), 5.82-5.69 (m, 1H), 4.81-4.70 (m, 2H), 3.79-3.56 (m, 6H), 3.53-3.37 (m, 3H), 3.29-3.18 (m, 1H), 2.39-2.19 (m, 1H), 2.19-2.06 (m, 1H).

### Example 18

### 5-{[2-{4-[5-Chloro-2-(2,2,2-trifluoroethoxy)phenyl]-5-methoxy-2-oxopyridin-1(2H)-yl}-3-(1,4-dioxan-2-yl)propanoyl]amino}pyridine-2-carboxamide (Diastereomer Mixture)

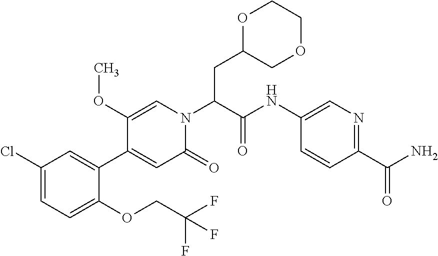 [SMILES: COc1cn(C(CC2COCCO2)C(=O)Nc2ccc(C(N)=O)nc2)c(=O)cc1-c1cc(Cl)ccc1OCC(F)(F)F]

40 mg (81 μmol) of 2-{4-[5-chloro-2-(2,2,2-trifluoroethoxy)phenyl]-5-methoxy-2-oxopyridin-1(2H)-yl}-3-(1,4-dioxan-2-yl)propanoic acid (diastereomer mixture) and 17.2 mg (122 μmol, 1.5 eq.) of 5-aminopyridine-2-carboxamide were reacted according to General Method 5. Yield: 26 mg (52% of theory)

LC/MS [Method 10]: Rₜ=1.57/1.60 min; MS (ESIpos): m/z=611/611 (M+H)⁺,

¹H-NMR (400 MHz, DMSO-d₆): δ [ppm]=10.84 (br. s., 1H), 8.83 (br. s., 1H), 8.25-8.18 (m, 1H), 8.05-7.94 (m, 2H), 7.56-7.46 (m, 2H), 7.40-7.30 (m, 2H), 7.29-7.24 (m, 1H), 6.35 and 6.33 (2×s, 1H), 5.80-5.66 (m, 1H), 4.81-4.71 (m, 2H), 3.80-3.37 (m, 9H), 3.28-3.19 (m, 1H), 2.40-2.22 (m, 1H), 2.22-2.06 (m, 1H).

### Example 19

### 5-{[2-{4-[5-Chloro-2-(2,2,2-trifluoroethoxy)phenyl]-5-methoxy-2-oxopyridin-1(2H)-yl}-3-(1,4-dioxan-2-yl)propanoyl]amino}-N-methylpyridine-2-carboxamide (Diastereomer Mixture)

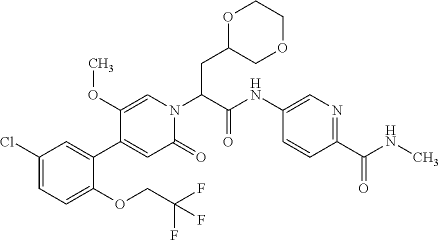 [SMILES: CNC(=O)c1ccc(NC(=O)C(CC2COCCO2)n2cc(OC)c(-c3cc(Cl)ccc3OCC(F)(F)F)cc2=O)cn1]

40 mg (81 μmol) of 2-{4-[5-chloro-2-(2,2,2-trifluoroethoxy)phenyl]-5-methoxy-2-oxopyridin-1(2H)-yl}-3-(1,4-dioxan-2-yl)propanoic acid (diastereomer mixture) and 18.8 mg (122 μmol, 1.5 eq.) of 5-amino-N-methylpyridine-2-carboxamide were reacted according to General Method 5. Yield: 35 mg (69% of theory)

LC/MS [Method 1]: Rₜ=0.90/0.92 min; MS (ESIpos): m/z=625/625 (M+H)⁺,

¹H-NMR (400 MHz, DMSO-d₆): δ [ppm]=10.84 and 10.81 (2×s, 1H), 8.89-8.83 (m, 1H), 8.70-8.61 (m, 1H), 8.24-8.17 (m, 1H), 8.03-7.97 (m, 1H), 7.52-7.47 (m, 1H), 7.37-7.30 (m, 2H), 7.29-7.24 (m, 1H), 6.35 and 6.33 (2×s, 1H), 5.80-5.66 (m, 1H), 4.81-4.71 (m, 2H), 3.81-3.35 (m, 9H), 3.28-3.19 (m, 1H), 2.80 (d, 3H), 2.40-2.22 (m, 1H), 2.22-2.06 (m, 1H).

### Example 20

### 4-[(2-{4-[5-Chloro-2-(2,2,2-trifluoroethoxy)phenyl]-5-methoxy-2-oxopyridin-1(2H)-yl}-3-[(2R)-tetrahydro-2H-pyran-2-yl]propanoyl)amino]benzoic acid (Diastereomer Mixture)

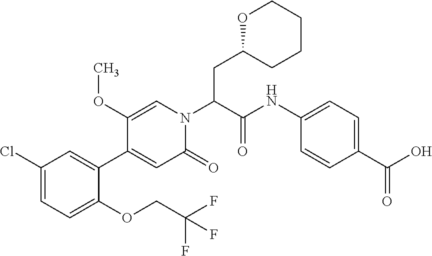 [SMILES: COc1cn(C(C[C@H]2CCCCO2)C(=O)Nc2ccc(C(=O)O)cc2)c(=O)cc1-c1cc(Cl)ccc1OCC(F)(F)F]

39 mg (63 μmol) of methyl 4-[(2-{4-[5-chloro-2-(2,2,2-trifluoroethoxy)phenyl]-5-methoxy-2-oxopyridin-1 (2H)-yl}-3-[(2R)-tetrahydro-2H-pyran-2-yl]propanoyl)amino]benzoate (diastereomer mixture) were reacted according to General Method 2. Yield: 25 mg (66% of theory)

LC/MS [Method 1]: Rₜ=1.04 min; MS (ESIpos): m/z=609 (M+H)⁺,

¹H-NMR (400 MHz, DMSO-d₆): δ [ppm]=12.73 (br. s., 1H), 10.71 and 10.61 (2×s, 1H), 7.94-7.85 (m, 2H), 7.80-7.70 (m, 2H), 7.52-7.46 (m, 1H), 7.39-7.29 (m, 2H), 7.29-7.20 (m, 1H), 6.33 and 6.30 (2×s, 1H), 5.85-5.65 (m, 1H), 4.83-4.67 (m, 2H), 3.91-3.76 (m, 1H), 3.60 and 3.60 (2×s, 3H), 3.28-2.99 (m, 2H), 2.41-2.11 (m, 2H), 1.80-1.70 (m, 1H), 1.68-1.51 (m, 1H), 1.48-1.18 (m, 4H).

### Example 21

### 2-{4-[5-Chloro-2-(2,2,2-trifluoroethoxy)phenyl]-5-methoxy-2-oxopyridin-1(2H)-yl}-N-(2-methyl-2H-indazol-5-yl)-3-[(2R)-tetrahydro-2H-pyran-2-yl]propanamide (Diastereomer Mixture)

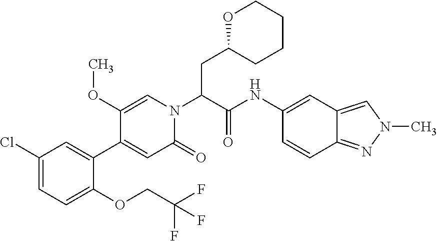 [SMILES: COc1cn(C(C[C@H]2CCCCO2)C(=O)Nc2ccc3nn(C)cc3c2)c(=O)cc1-c1cc(Cl)ccc1OCC(F)(F)F]

25 mg (51 μmol) of 2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoic acid (diastereomer mixture) and 13.5 mg (92 μmol, 1.8 eq.) of 2-methyl-2H-indazole-5-amine were reacted according to General Method 5. Yield: 23 mg (73% of theory)

LC/MS [Method 10]: Rₜ=1.93/1.96 min; MS (ESIpos): m/z=619/619 (M+H)⁺,

¹H-NMR (400 MHz, DMSO-d₆): δ [ppm]=10.36 and 10.27 (2×s, 1H), 8.29-8.19 (m, 1H), 8.16-8.05 (m, 1H), 7.58-7.46 (m, 2H), 7.42-7.29 (m, 3H), 7.29-7.23 (m, 1H), 6.33 and 6.31 (2×s, 1H), 5.88-5.68 (m, 1H), 4.84-4.66 (m, 2H), 4.13 and 4.12 (2×s, 3H), 3.93-3.78 (m, 1H), 3.60 and 3.60 (2×s, 3H), 3.22-2.98 (m, 2H), 2.39-2.10 (m, 2H), 1.81-1.52 (m, 2H), 1.48-1.17 (m, 4H).

### Example 22

### 5-[(2-{4-[5-Chloro-2-(2,2,2-trifluoroethoxy)phenyl]-5-methoxy-2-oxopyridin-1(2H)-yl}-3-[(2R)-tetrahydro-2H-pyran-2-yl]propanoyl)amino]-N-methylpyridine-2-carboxamide (Diastereomer Mixture)

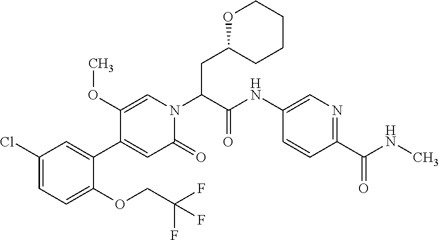 [SMILES: CNC(=O)c1ccc(NC(=O)C(C[C@H]2CCCCO2)n2cc(OC)c(-c3cc(Cl)ccc3OCC(F)(F)F)cc2=O)cn1]

25 mg (51 μmol) of 2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoic acid (diastereomer mixture) and 11.9 mg (77 μmol, 1.5 eq.) of 5-amino-N-methylpyridine-2-carboxamide were reacted according to General Method 5. Yield: 25 mg (77% of theory)

LC/MS [Method 10]: Rₜ=1.91/1.93 min; MS (ESIpos): m/z=623/623 (M+H)⁺,

¹H-NMR (400 MHz, DMSO-d₆): δ [ppm]=10.84 and 10.74 (2×s, 1H), 8.89-8.84 (m, 1H), 8.69-8.60 (m, 1H), 8.26-8.14 (m, 1H), 8.04-7.92 (m, 1H), 7.54-7.43 (m, 1H), 7.37-7.28 (m, 2H), 7.28-7.21 (m, 1H), 6.33 and 6.31 (2×s, 1H), 5.82-5.63 (m, 1H), 4.81-4.69 (m, 2H), 3.90-3.75 (m, 1H), 3.60 and 3.60 (2×s, 3H), 3.23-2.96 (m, 2H), 2.80 (d, 3H), 2.43-2.13 (m, 2H), 1.80-1.51 (m, 2H), 1.48-1.18 (m, 4H).

### Example 23

### 4-[(2-{4-[5-Chloro-2-(2,2,2-trifluoroethoxy)phenyl]-5-methoxy-2-oxopyridin-1(2H)-yl}-3-[(2R)-tetrahydro-2H-pyran-2-yl]propanoyl)amino]-2-fluorobenzamide (Diastereomer Mixture)

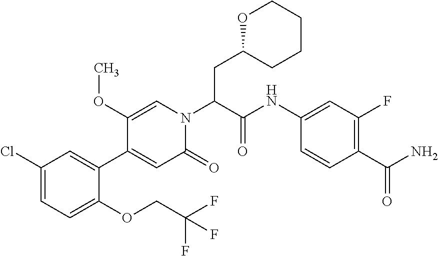 [SMILES: COc1cn(C(C[C@H]2CCCCO2)C(=O)Nc2ccc(C(N)=O)c(F)c2)c(=O)cc1-c1cc(Cl)ccc1OCC(F)(F)F]

25 mg (51 μmol) of 2-{4-[5-chloro-2-(2,2,2-trifluoroethoxy)phenyl]-5-methoxy-2-oxopyridin-1(2H)-yl}-3-[(2R)-tetrahydro-2H-pyran-2-yl]propanoic acid (diastereomer mixture) and 12.2 mg (77 μmol, 1.5 eq.) of 4-amino-2-fluorobenzamide were reacted according to General Method 5. Yield: 20 mg (63% of theory)

LC/MS [Method 10]: Rₜ=1.91/1.89 min; MS (ESIpos): m/z=626/626 (M+H)⁺,

¹H-NMR (400 MHz, DMSO-d₆): δ [ppm]=10.77 and 10.67 (2×s, 1H), 7.71-7.62 (m, 2H), 7.56-7.47 (m, 3H), 7.47-7.41 (m, 1H), 7.36-7.28 (m, 2H), 7.28-7.23 (m, 1H), 6.33 and 6.31 (2×s, 1H), 5.80-5.63 (m, 1H), 4.81-4.69 (m, 2H), 3.90-3.78 (m, 1H), 3.60 (s, 3H), 3.27-2.97 (m, 2H), 2.41-2.11 (m, 2H), 1.80-1.71 (m, 1H), 1.66-1.51 (m, 1H), 1.47-1.18 (m, 4H).

### Example 24

### 2-{4-[5-Chloro-2-(2,2,2-trifluoroethoxy)phenyl]-5-methoxy-2-oxopyridin-1(2H)-yl}-3-(5,6-dihydro-4H-1,2-oxazin-3-yl)-N-(2-methyl-2H-indazol-5-yl)propanamide (Racemate)

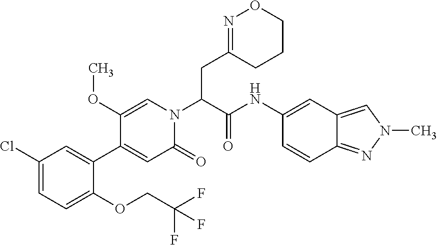 [SMILES: COc1cn(C(CC2=NOCCC2)C(=O)Nc2ccc3nn(C)cc3c2)c(=O)cc1-c1cc(Cl)ccc1OCC(F)(F)F]

18 mg (26 μmol, 70% purity) of 2-{4-[5-chloro-2-(2,2,2-trifluoroethoxy)phenyl]-5-methoxy-2-oxopyridin-1(2H)-yl}-3-(5,6-dihydro-4H-1,2-oxazin-3-yl)propanoic acid (racemate) and 6.4 mg (39 μmol, 89% purity, 1.5 eq.) of 2-methyl-2H-indazole-5-amine were reacted according to General Method 5. Yield: 5.8 mg (31% of theory)

LC/MS [Method 10]: Rₜ=1.73 min; MS (ESIpos): m/z=618 (M+H)⁺,

¹H-NMR (400 MHz, DMSO-d₆): δ [ppm]=10.40 (s, 1H), 8.26 (s, 1H), 8.12 (s, 1H), 7.55 (d, 1H), 7.49 (dd, 1H), 7.35-7.24 (m, 5H), 6.35 (s, 1H), 5.96 (dd, 1H), 4.81-4.71 (m, 2H), 4.13 (s, 3H), 3.81-3.72 (m, 1H), 3.72-3.63 (m, 1H), 3.59 (s, 3H), 3.14-2.84 (m, 2H), 2.23-2.15 (m, 2H), 1.86-1.69 (m, 2H).

### Example 25

### 4-[(4-tert-Butoxy-2-{4-[5-chloro-2-(2,2-difluoroethoxy)phenyl]-5-methoxy-2-oxopyridin-1(2H)-yl}butanoyl)amino]benzoic acid (Racemate)

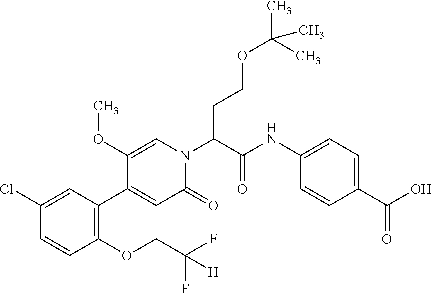 [SMILES: COc1cn(C(CCOC(C)(C)C)C(=O)Nc2ccc(C(=O)O)cc2)c(=O)cc1-c1cc(Cl)ccc1OCC(F)F]

48 mg (0.079 mmol) of methyl 4-[(4-tert-butoxy-2-{4-[5-chloro-2-(2,2-difluoroethoxy)phenyl]-5-methoxy-2-oxopyridin-1(2H)-yl}butanoyl)amino]benzoate (racemate) were reacted according to General Method 2. 15 eq. of lithium hydroxide were used in the reaction. Yield: 27.5 mg (59% of theory).

LC/MS [Method 10]: Rₜ=1.95 min; MS (ESIpos): m/z=593 (M+H)⁺,

¹H-NMR (400 MHz, DMSO-d₆): δ [ppm]=12.73 (brs, 1H), 10.70 (s, 1H), 7.92-7.87 (m, 2H), 7.79-7.73 (m, 2H), 7.47-7.43 (m, 1H), 7.34 (s, 1H), 7.27-7.20 (m, 2H), 6.32 (s, 1H), 6.20 (tt, 1H), 5.80-5.73 (m, 1H), 4.41-4.24 (m, 2H), 3.62 (s, 3H), 3.42-3.35 (m, 1H), 3.29-3.23 (m, 1H, partly concealed), 2.40-2.28 (m, 2H), 1.04 (s, 9H).

### Example 26

### 4-[(4-tert-Butoxy-2-{4-[5-chloro-2-(2,2-difluoroethoxy)phenyl]-5-methoxy-2-oxopyridin-1(2H)-yl}butanoyl)amino]-2-fluorobenzamide (Racemate)

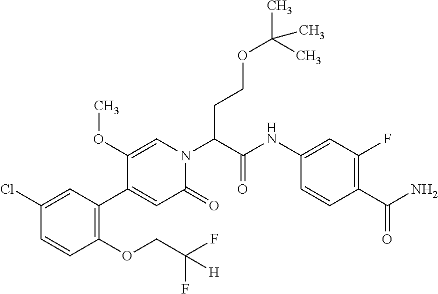 [SMILES: COc1cn(C(CCOC(C)(C)C)C(=O)Nc2ccc(C(N)=O)c(F)c2)c(=O)cc1-c1cc(Cl)ccc1OCC(F)F]

30 mg (0.063 mmol) of 4-tert-butoxy-2-{4-[5-chloro-2-(2,2-difluoroethoxy)phenyl]-5-methoxy-2-oxopyridin-1(2H)-yl}butanoic acid (racemate) and 14.6 mg (0.095 mmol, 1.5 eq.) of 4-amino-2-fluorobenzamide were reacted according to General Method 5. Yield: 34 mg (88% of theory).

LC/MS [Method 10]: Rₜ=1.91 min; MS (ESIpos): m/z=610 (M+H)⁺,

¹H-NMR (400 MHz, DMSO-d₆): δ [ppm]=10.78 (s, 1H), 7.71 (m, 2H), 7.55-7.50 (m, 2H), 7.48-7.43 (m, 2H), 7.33 (s, 1H), 7.28-7.21 (m, 2H), 6.33 (s, 1H), 6.21 (tt, 1H), 5.77-5.70 (m, 1H), 4.40-4.26 (m, 2H), 3.62 (s, 3H), 3.42-3.36 (m, 1H), 3.30-3.24 (m, 1H, partly concealed), 2.39-2.32 (m, 2H), 1.04 (s, 9H).

### Example 27

### 5-[(4-tert-Butoxy-2-{4-[5-chloro-2-(2,2-difluoroethoxy)phenyl]-5-methoxy-2-oxopyridin-1(2H)-yl}butanoyl)amino]pyridine-2-carboxamide (Racemate)

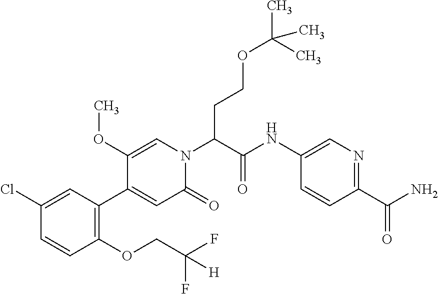 [SMILES: COc1cn(C(CCOC(C)(C)C)C(=O)Nc2ccc(C(N)=O)nc2)c(=O)cc1-c1cc(Cl)ccc1OCC(F)F]

30 mg (0.063 mmol) of 4-tert-butoxy-2-{4-[5-chloro-2-(2,2-difluoroethoxy)phenyl]-5-methoxy-2-oxopyridin-1(2H)-yl}butanoic acid (racemate) and 13.0 mg (0.095 mmol, 1.5 eq.) of 5-aminopyridine-2-carboxamide were reacted according to General Method 5. Yield: 33 mg (88% of theory).

LC/MS [Method 10]: Rₜ=1.85 min; MS (ESIpos): m/z=593 (M+H)⁺,

¹H-NMR (400 MHz, DMSO-d₆): δ [ppm]=10.87 (s, 1H), 8.89-8.86 (m, 1H), 8.27-8.22 (m, 2H), 8.04-7.99 (m, 2H), 7.54-7.50 (m, 1H), 7.48-7.44 (m, 1H), 7.34 (s, 1H), 7.27 (d, 1H), 7.23 (d, 1H), 6.34 (s, 1H), 6.21 (tt, 1H), 5.80-5.74 (m, 1H), 4.41-4.25 (m, 2H), 3.62 (s, 3H), 3.44-3.37 (m, 1H), 3.30-3.25 (m, 1H, partly concealed), 2.43-2.32 (m, 1H), 1.05 (s, 9H).

### Example 28

### 4-tert-Butoxy-2-{4-[5-chloro-2-(2,2-difluoroethoxy)phenyl]-5-methoxy-2-oxopyridin-1(2H)-yl}-N-(2-methyl-2H-indazol-5-yl)butanamide (Racemate)

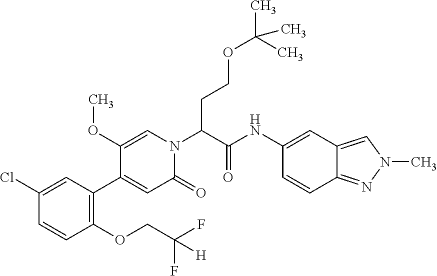 [SMILES: COc1cn(C(CCOC(C)(C)C)C(=O)Nc2ccc3nn(C)cc3c2)c(=O)cc1-c1cc(Cl)ccc1OCC(F)F]

30 mg (0.063 mmol) of 4-tert-butoxy-2-{4-[5-chloro-2-(2,2-difluoroethoxy)phenyl]-5-methoxy-2-oxopyridin-1(2H)-yl}butanoic acid (racemate) and 14.0 mg (0.095 mmol, 1.5 eq.) of 2-methyl-2H-indazole-5-amine were reacted according to General Method 5. Yield: 35 mg (92% of theory).

LC/MS [Method 10]: Rₜ=1.96 min; MS (ESIpos): m/z=603 (M+H)⁺,

¹H-NMR (400 MHz, DMSO-d₆): δ [ppm]=10.34 (s, 1H), 8.25 (s, 1H), 8.15-8.12 (m, 1H), 7.54 (d, 1H), 7.48-7.43 (m, 1H), 7.38 (s, 1H), 7.34-7.29 (m, 1H), 7.27-7.20 (m, 2H), 6.32 (s, 1H), 6.21 (tt, 1H), 5.81-5.75 (m, 1H), 4.41-4.25 (m, 2H), 4.13 (s, 3H), 3.62 (s, 3H), 3.42-3.34 (m, 1H), 2.38-2.24 (m, 2H), 1.05 (s, 9H).

### Example 29

### 4-{[2-{4-[5-Chloro-2-(2,2-difluoroethoxy)phenyl]-5-methoxy-2-oxopyridin-1(2H)-yl}-3-(1,4-dioxan-2-yl)propanoyl]amino}benzoic acid (Diastereomer Mixture)

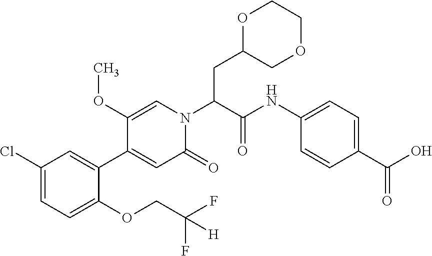 [SMILES: COc1cn(C(CC2COCCO2)C(=O)Nc2ccc(C(=O)O)cc2)c(=O)cc1-c1cc(Cl)ccc1OCC(F)F]

48 mg (79 μmol) of methyl 4-{[2-{4-[5-chloro-2-(2,2-difluoroethoxy)phenyl]-5-methoxy-2-oxopyridin-1(2H)-yl}-3-(1,4-dioxan-2-yl)propanoyl]amino}benzoate (diastereomer mixture) were reacted according to General Method 2. Yield: 28 mg (60% of theory)

LC/MS [Method 2]: Rₜ=2.79/2.85 min; MS (ESIpos): m/z=593/593 (M+H)⁺,

¹H-NMR (400 MHz, DMSO-d₆): δ [ppm]=12.76 (bs, 1H), 10.72 and 10.69 (2×s, 1H), 7.97-7.84 (m, 2H), 7.79-7.71 (dd, 2H), 7.50-7.41 (m, 1H), 7.38-7.29 (m, 2H), 7.24 and 7.22 (2×s, 1H), 6.38-6.06 (m, 2H), 5.86-5.66 (m, 1H), 4.39-4.27 (m, 2H), 3.79-3.58 (m, 6H), 3.58-3.39 (m, 3H), 3.28-3.18 (m, 1H), 2.35-2.20 (m, 1H), 2.20-2.04 (m, 1H).

### Example 30

### 5-{[2-{4-[5-Chloro-2-(2,2-difluoroethoxy)phenyl]-5-methoxy-2-oxopyridin-1(2H)-yl}-3-(1,4-dioxan-2-yl)propanoyl]amino}pyridine-2-carboxamide (Diastereomer Mixture)

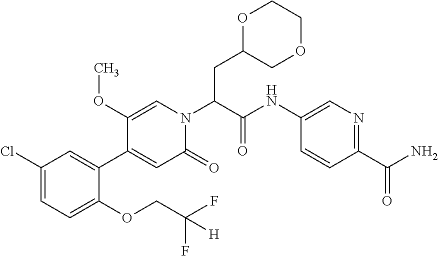 [SMILES: COc1cn(C(CC2COCCO2)C(=O)Nc2ccc(C(N)=O)nc2)c(=O)cc1-c1cc(Cl)ccc1OCC(F)F]

40 mg (80% purity, 68 μmol) of 2-{4-[5-chloro-2-(2,2-difluoroethoxy)phenyl]-5-methoxy-2-oxopyridin-1(2H)-yl}-3-(1,4-dioxan-2-yl)propanoic acid (diastereomer mixture) and 14.3 mg (101 μmol, 1.5 eq.) of 5-aminopyridine-2-carboxamide were reacted according to General Method 5. Yield: 31 mg (77% of theory)

LC/MS [Method 2]: Rₜ=2.51/2.57 min; MS (ESIpos): m/z=593/593 (M+H)⁺,

¹H-NMR (400 MHz, DMSO-d₆): δ [ppm]=10.87 and 10.84 (2×s, 1H), 8.87-8.83 (m, 1H), 8.27-8.18 (m, 1H), 8.05-7.97 (m, 2H), 7.56-7.49 (m, 1H), 7.48-7.43 (m, 1H), 7.38-7.28 (m, 2H), 7.26-7.20 (m, 1H), 6.38-6.06 (m, 2H), 5.80-5.68 (m, 1H), 4.39-4.28 (m, 2H), 3.80-3.58 (m, 6H), 3.56-3.34 (m, 3H), 3.28-3.20 (m, 1H), 2.38-2.24 (m, 1H), 2.22-2.05 (m, 1H).

### Example 31

### 5-{[2-{4-[5-Chloro-2-(2,2-difluoroethoxy)phenyl]-5-methoxy-2-oxopyridin-1(2H)-yl}-3-(1,4-dioxan-2-yl)propanoyl]amino}-N-methylpyridine-2-carboxamide (Diastereomer Mixture)

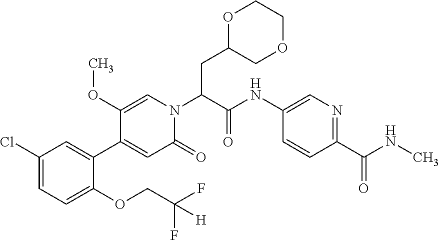 [SMILES: CNC(=O)c1ccc(NC(=O)C(CC2COCCO2)n2cc(OC)c(-c3cc(Cl)ccc3OCC(F)F)cc2=O)cn1]

40 mg (80% purity, 68 μmol) of 2-{4-[5-chloro-2-(2,2-difluoroethoxy)phenyl]-5-methoxy-2-oxopyridin-1(2H)-yl}-3-(1,4-dioxan-2-yl)propanoic acid (diastereomer mixture) and 15.6 mg (101 mol, 1.5 eq.) of 5-amino-N-methylpyridine-2-carboxamide were reacted according to General Method 5. Yield: 35 mg (85% of theory)

LC/MS [Method 2]: Rₜ=2.66/2.72 min; MS (ESIpos): m/z=607/607 (M+H)⁺,

¹H-NMR (400 MHz, DMSO-d₆): δ [ppm]=10.85 and 10.82 (2×s, 1H), 8.92-8.81 (m, 1H), 8.70-8.60 (m, 1H), 8.27-8.15 (m, 1H), 8.05-7.93 (m, 1H), 7.49-7.42 (m, 1H), 7.37-7.28 (m, 2H), 7.26-7.19 (m, 1H), 6.39-6.05 (m, 2H), 5.81-5.61 (m, 1H), 4.40-4.25 (td, 2H), 3.81-3.39 (m, 9H), 3.28-3.18 (m, 2H), 2.80 (d, 3H), 2.38-2.25 (m, 1H), 2.23-2.04 (m, 1H).

### Example 32

### 4-{[2-{4-[5-Chloro-2-(2,2-difluoroethoxy)phenyl]-5-methoxy-2-oxopyridin-1(2H)-yl}-3-(1,4-dioxan-2-yl)propanoyl]amino}-2-fluorobenzamide (Diastereomer Mixture)

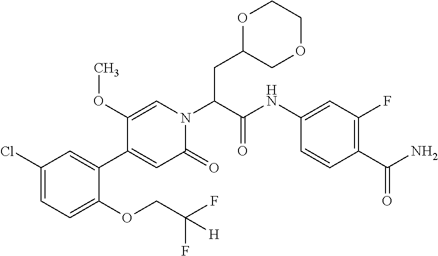 [SMILES: COc1cn(C(CC2COCCO2)C(=O)Nc2ccc(C(N)=O)c(F)c2)c(=O)cc1-c1cc(Cl)ccc1OCC(F)F]

40 mg (80% purity, 68 μmol) of 2-{4-[5-chloro-2-(2,2-difluoroethoxy)phenyl]-5-methoxy-2-oxopyridin-1(2H)-yl}-3-(1,4-dioxan-2-yl)propanoic acid (diastereomer mixture) and 16.1 mg (101 μmol, 1.5 eq.) of 4-amino-2-fluorobenzamide were reacted according to General Method 5. Yield: 18 mg (44% of theory)

LC/MS [Method 2]: Rₜ=2.65/2.70 min; MS (ESIpos): m/z=610/610 (M+H)⁺,

¹H-NMR (400 MHz, DMSO-d₆): δ [ppm]=10.78-10.75 (2×s, 1H), 7.72-7.61 (m, 2H), 7.57-7.49 (m, 2H), 7.49-7.40 (m, 2H), 7.37-7.28 (m, 2H), 7.25-7.20 (m, 1H), 6.38-6.06 (m, 2H), 5.77-5.66 (m, 1H), 4.39-4.28 (m, 2H), 3.77-3.38 (m, 9H), 3.28-3.19 (m, 1H), 2.35-2.21 (m, 1H), 2.19-2.02 (m, 1H).

### Example 33

### 2-{4-[5-Chloro-2-(2,2-difluoroethoxy)phenyl]-5-methoxy-2-oxopyridin-1(2H)-yl}-3-(1,4-dioxan-2-yl)-N-(2-methyl-2H-indazol-5-yl)propanamide (Diastereomer Mixture)

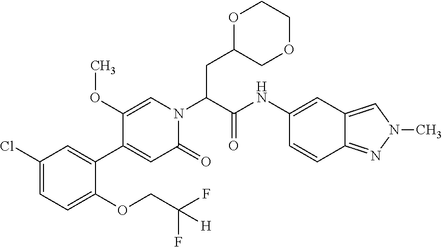 [SMILES: COc1cn(C(CC2COCCO2)C(=O)Nc2ccc3nn(C)cc3c2)c(=O)cc1-c1cc(Cl)ccc1OCC(F)F]

40 mg (80% purity, 68 μmol) of 2-{4-[5-chloro-2-(2,2-difluoroethoxy)phenyl]-5-methoxy-2-oxopyridin-1(2H)-yl}-3-(1,4-dioxan-2-yl)propanoic acid (diastereomer mixture) and 15.2 mg (101 mol, 1.5 eq.) of 2-methyl-2H-indazole-5-amine were reacted according to General Method 5. Yield: 33 mg (81% of theory)

LC/MS [Method 2]: Rₜ=2.72/2.78 min; MS (ESIpos): m/z=603/603 (M+H)⁺,

¹H-NMR (400 MHz, DMSO-d₆): δ [ppm]=10.39 and 10.35 (2×s, 1H), 8.29-8.21 (m, 1H), 8.16-8.07 (m, 1H), 7.58-7.50 (m, 1H), 7.48-7.43 (m, 1H), 7.43-7.35 (m, 1H), 7.34-7.28 (m, 2H), 7.25-7.20 (m, 1H), 6.38-6.07 (m, 2H), 5.85-5.73 (m, 1H), 4.39-4.27 (m, 2H), 4.13 and 4.13 (2×s, 3H), 3.80-3.40 (m, 9H), 3.39-3.34 (m, 1H), 3.28-3.19 (m, 1H), 2.35-2.19 (m, 1H), 2.19-2.00 (m, 1H).

### Example 34

### 4-[(2-{4-[5-Chloro-2-(2,2,2-trifluoroethoxy)phenyl]-5-methoxy-2-oxopyridin-1(2H)-yl}-4-isopropoxybutanoyl)amino]benzoic acid (Racemate)

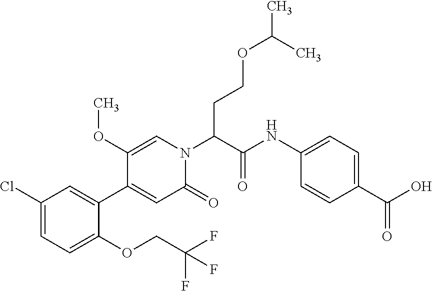 [SMILES: COc1cn(C(CCOC(C)C)C(=O)Nc2ccc(C(=O)O)cc2)c(=O)cc1-c1cc(Cl)ccc1OCC(F)(F)F]

To 106 mg (0.17 mmol) of methyl 4-[(2-{4-[5-chloro-2-(2,2,2-trifluoroethoxy)phenyl]-5-methoxy-2-oxopyridin-1(2H)-yl}-4-isopropoxybutanoyl)amino]benzoate (racemate) in 5.0 ml of tetrahydrofuran was added 0.87 ml of lithium hydroxide solution (1 N in water), and the mixture was stirred at RT for 2 days. The reaction mixture was diluted with 3.0 ml of acetonitrile/water mixture (1:1) and separated by means of preparative RP-HPLC (water/acetonitrile gradient with 0.1% formic acid). The crude product obtained after lyophilization was dissolved in acetonitrile, and the solution was adjusted to pH 3 with 1 N hydrochloric acid and separated again by means of preparative RP-HPLC (water/acetonitrile gradient with 0.1% formic acid). Yield: 74 mg (71% of theory)

LC/MS [Method 10]: Rₜ=1.94 min; MS (ESIpos): m/z=597 (M+H)⁺,

¹H-NMR (400 MHz, DMSO-d₆): δ [ppm]=10.70-10.66 (m, 1H), 7.89 (s, 2H), 7.80-7.73 (m, 2H), 7.53-7.46 (m, 1H), 7.36-7.29 (m, 2H), 7.28-7.23 (m, 1H), 6.32 (s, 1H), 5.81-5.73 (m, 1H), 4.83-4.67 (m, 2H), 3.60 (s, 3H), 3.47-3.36 (m, 2H), 3.28-3.21 (m, 1H), 2.42-2.31 (m, 2H), 1.02 (d, 3H), 0.99-0.95 (m, 3H).

### Example 35

### 4-{[(4S)-2-{4-[5-Chloro-2-(2,2,2-trifluoroethoxy)phenyl]-5-methoxy-2-oxopyridin-1(2H)-yl}-4-methoxypentanoyl]amino}benzoic acid (Racemate)

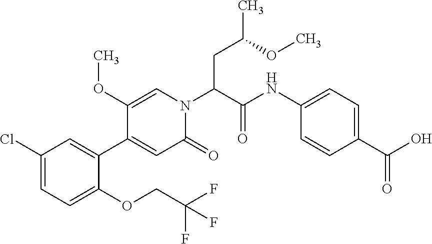 [SMILES: COc1cn(C(C[C@H](C)OC)C(=O)Nc2ccc(C(=O)O)cc2)c(=O)cc1-c1cc(Cl)ccc1OCC(F)(F)F]

100 mg (0.22 mmol) of (4S)-2-[4-[5-chloro-2-(2,2,2-trifluoroethoxy)phenyl]-5-methoxy-2-oxo-1-pyridyl]-4-methoxypentanoic acid (racemate) and 32.5 mg (0.24 mmol, 1.1 eq.) of 4-aminobenzoic acid were reacted according to General Method 5. Yield: 27 mg (21% of theory)

LC/MS [Method 1]: Rₜ=0.99 min; MS (ESIpos): m/z=583 (M+H)⁺,

¹H-NMR (400 MHz, DMSO-d₆): δ [ppm]=12.76 (br. s., 1H), 10.78-10.63 (m, 1H), 7.95-7.84 (m, 2H), 7.81-7.70 (m, 2H), 7.52-7.46 (m, 1H), 7.44-7.31 (m, 2H), 7.29-7.21 (m, 1H), 6.33 (s, 1H), 5.87-5.76 (m, 1H), 4.81-4.68 (m, 2H), 3.60 (s, 3H), 3.20-3.02 (m, 4H), 2.41-2.29 (m, 1H), 2.25-2.09 (m, 1H), 1.17-1.09 (m, 3H).

### Example 36

### 5-{[(4S)-2-{4-[5-Chloro-2-(2,2,2-trifluoroethoxy)phenyl]-5-methoxy-2-oxopyridin-1(2H)-yl}-4-methoxypentanoyl]amino}-N-methylpyridine-2-carboxamide (Racemate)

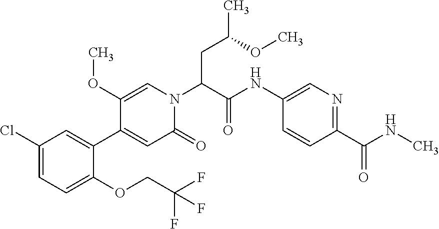 [SMILES: CNC(=O)c1ccc(NC(=O)C(C[C@H](C)OC)n2cc(OC)c(-c3cc(Cl)ccc3OCC(F)(F)F)cc2=O)cn1]

100 mg (0.22 mmol) of (4S)-2-[4-[5-chloro-2-(2,2,2-trifluoroethoxy)phenyl]-5-methoxy-2-oxo-1-pyridyl]-4-methoxypentanoic acid (racemate) and 36 mg (0.24 mmol, 1.1 eq.) of 5-amino-N-methylpyridine-2-carboxamide were reacted according to General Method 5. Yield: 32 mg (25% of theory)

LC/MS [Method 1]: Rₜ=0.98 min; MS (ESIpos): m/z=597 (M+H)⁺,

¹H-NMR (400 MHz, DMSO-d₆): δ [ppm]=10.90-10.78 (m, 1H), 8.91-8.86 (m, 1H), 8.70-8.61 (m, 1H), 8.26-8.19 (m, 1H), 8.03-7.97 (m, 1H), 7.53-7.46 (m, 1H), 7.43-7.31 (m, 2H), 7.28-7.23 (m, 1H), 6.37-6.31 (m, 1H), 5.86-5.75 (m, 1H), 4.81-4.69 (m, 2H), 3.61 (s, 3H), 3.10 (s, 4H), 2.84-2.76 (m, 3H), 2.40-2.30 (m, 1H), 2.27-2.17 (m, 1H), 1.19-1.08 (m, 3H).

### Example 37

### (4S)-2-{4-[5-Chloro-2-(2,2,2-trifluoroethoxy)phenyl]-5-methoxy-2-oxopyridin-1(2H)-yl}-4-methoxy-N-(2-methyl-2H-indazol-5-yl)pentanamide (Racemate)

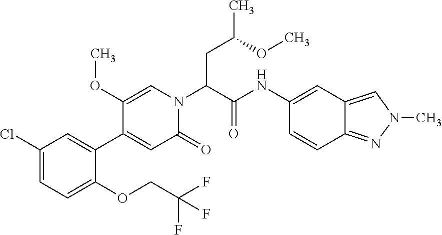 [SMILES: COc1cn(C(C[C@H](C)OC)C(=O)Nc2ccc3nn(C)cc3c2)c(=O)cc1-c1cc(Cl)ccc1OCC(F)(F)F]

100 mg (0.22 mmol) of (4S)-2-[4-[5-chloro-2-(2,2,2-trifluoroethoxy)phenyl]-5-methoxy-2-oxo-1-pyridyl]-4-methoxypentanoic acid (racemate) and 39 mg (89% purity, 0.24 mmol, 1.1 eq.) of 2-methyl-2H-indazole-5-amine were reacted according to General Method 5. Yield: 23 mg (18% of theory)

LC/MS [Method 1]: Rₜ=1.02 min; MS (ESIpos): m/z=593 (M+H)⁺,

¹H-NMR (400 MHz, DMSO-d₆): δ [ppm]=10.43-10.30 (m, 1H), 8.27-8.22 (m, 1H), 8.15-8.10 (m, 1H), 7.57-7.44 (m, 2H), 7.40-7.29 (m, 3H), 7.29-7.22 (m, 1H), 6.36-6.30 (m, 1H), 5.88-5.78 (m, 1H), 4.81-4.69 (m, 2H), 4.13 (s, 3H), 3.61 (s, 3H), 3.11 (s, 4H), 2.36-2.25 (m, 1H), 2.24-2.10 (m, 1H), 1.18-1.09 (m, 3H).

### Example 38

### 2-{4-[5-Chloro-2-(2,2,2-trifluoroethoxy)phenyl]-5-methoxy-2-oxopyridin-1(2H)-yl}-N-(2-methyl-2H-indazol-5-yl)-4-(trifluoromethoxy)butanamide (Racemate)

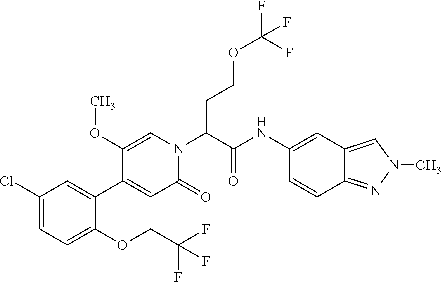 [SMILES: COc1cn(C(CCOC(F)(F)F)C(=O)Nc2ccc3nn(C)cc3c2)c(=O)cc1-c1cc(Cl)ccc1OCC(F)(F)F]

60 mg (0.12 mmol) of 2-{4-[5-chloro-2-(2,2,2-trifluoroethoxy)phenyl]-5-methoxy-2-oxopyridin-1(2H)-yl}-4-(trifluoromethoxy)butanoic acid (racemate) and 29.5 mg (89% purity, 0.18 mmol, 1.5 eq.) of 2-methyl-2H-indazole-5-amine were reacted according to General Method 5. Yield: 50 mg (67% of theory)

LC/MS [Method 1]: Rₜ=1.04 min; MS (ESIpos): m/z=633 (M+H)⁺,

¹H-NMR (400 MHz, DMSO-d₆): δ [ppm]=10.39 (s, 1H), 8.26 (s, 1H), 8.11 (s, 1H), 7.55 (d, 1H), 7.46-7.52 (m, 1H), 7.38 (s, 1H), 7.34-7.23 (m, 3H), 6.35 (s, 1H), 5.86-5.77 (m, 1H), 4.80-4.69 (m, 2H), 4.17-4.07 (m, 4H), 3.99-3.90 (m, 1H), 3.60 (s, 3H), 2.64-2.55 (m, 2H).

### Example 39

### 2-{4-[5-Chloro-2-(2,2,2-trifluoroethoxy)phenyl]-5-methoxy-2-oxopyridin-1(2H)-yl}-4-isopropoxy-N-(2-methyl-2H-indazol-5-yl)butanamide (Racemate)

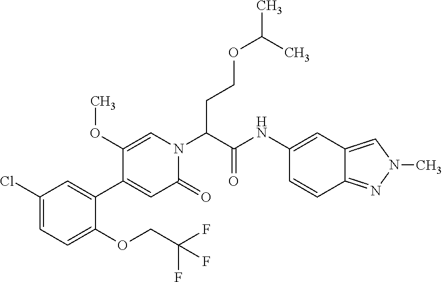 [SMILES: COc1cn(C(CCOC(C)C)C(=O)Nc2ccc3nn(C)cc3c2)c(=O)cc1-c1cc(Cl)ccc1OCC(F)(F)F]

100 mg (0.21 mmol) of 2-{4-[5-chloro-2-(2,2,2-trifluoroethoxy)phenyl]-5-methoxy-2-oxopyridin-1(2H)-yl}-4-isopropoxybutanoic acid (racemate) and 51.9 mg (89% purity, 0.31 mmol, 1.5 eq.) of 2-methyl-2H-indazole-5-amine were reacted according to General Method 5. Yield: 98 mg (73% of theory)

LC/MS [Method 1]: Rₜ=1.95 min; MS (ESIpos): m/z=607 (M+H)⁺,

¹H-NMR (400 MHz, DMSO-d₆): δ [ppm]=10.33 (s, 1H), 8.25 (s, 1H), 8.15-8.12 (m, 1H), 7.56-7.52 (m, 1H), 7.46-7.51 (m, 1H), 7.38 (s, 1H), 7.29-7.34 (m, 3H), 7.26 (d, 1H), 6.33 (s, 1H), 5.75-5.83 (m, 1H), 4.68-4.83 (m, 3H), 3.60 (s, 3H), 3.35-3.48 (m, 3H), 3.22-3.28 (m, 1H), 2.31-2.42 (m, 2H), 1.01 (dd, 6H).

### Example 40

### 4-{[(4S)-2-{4-[5-Chloro-2-(2,2,2-trifluoroethoxy)phenyl]-5-methoxy-2-oxopyridin-1(2H)-yl}-4-methoxypentanoyl]amino}-2-fluorobenzamide (Racemate)

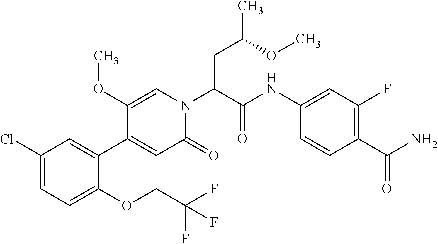 [SMILES: COc1cn(C(C[C@H](C)OC)C(=O)Nc2ccc(C(N)=O)c(F)c2)c(=O)cc1-c1cc(Cl)ccc1OCC(F)(F)F]

100 mg (0.22 mmol) of (4S)-2-[4-[5-chloro-2-(2,2,2-trifluoroethoxy)phenyl]-5-methoxy-2-oxo-1-pyridyl]-4-methoxypentanoic acid (racemate) and 36.6 mg (0.24 mmol, 1.1 eq.) of 4-amino-2-fluorobenzamide were reacted according to General Method 5. Yield: 22 mg (17% of theory)

LC/MS [Method 10]: Rₜ=1.78 min; MS (ESIpos): m/z=600 (M+H)⁺,

¹H-NMR (400 MHz, DMSO-d₆): δ [ppm]=10.82-10.73 (m, 1H), 7.72-7.62 (m, 2H), 7.57-7.38 (m, 5H), 7.36-7.31 (m, 2H), 7.29-7.21 (m, 2H), 6.35-6.31 (m, 1H), 5.81-5.74 (m, 1H), 4.81-4.69 (m, 2H), 3.60 (s, 3H), 3.20-3.01 (m, 4H), 2.39-2.31 (m, 1H), 2.23-2.11 (m, 1H), 1.17-1.08 (m, 3H).

### Example 41

### 4-[(2-{4-[5-Chloro-2-(2,2,2-trifluoroethoxy)phenyl]-5-methoxy-2-oxopyridin-1(2H)-yl}-4-isopropoxybutanoyl)amino]-2-fluorobenzamide (Racemate)

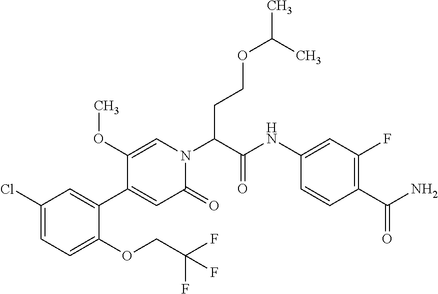 [SMILES: COc1cn(C(CCOC(C)C)C(=O)Nc2ccc(C(N)=O)c(F)c2)c(=O)cc1-c1cc(Cl)ccc1OCC(F)(F)F]

100 mg (0.21 mmol) of 2-{4-[5-chloro-2-(2,2,2-trifluoroethoxy)phenyl]-5-methoxy-2-oxopyridin-1(2H)-yl}-4-isopropoxybutanoic acid (racemate) and 54.4 mg (89% purity, 0.31 mmol, 1.5 eq.) of 4-amino-2-fluorobenzamide were reacted according to General Method 5. Yield: 109 mg (85% of theory)

LC/MS [Method 1]: Rₜ=1.01 min; MS (ESIpos): m/z=614 (M+H)⁺,

¹H-NMR (400 MHz, DMSO-d₆): δ [ppm]=10.75 (s, 1H), 7.71-7.63 (m, 2H), 7.56-7.46 (m, 3H), 7.46-7.41 (m, 1H), 7.34-7.30 (m, 2H), 7.26 (d, 1H), 6.33 (s, 1H), 5.77-5.70 (m, 1H), 4.81-4.69 (m, 2H), 3.60 (s, 3H), 3.48-3.36 (m, 2H), 3.27-3.19 (m, 1H), 2.41-2.33 (m, 2H), 1.00 (dd, 6H).

### Example 42

### 4-{[2-{4-[5-Chloro-2-(2,2,2-trifluoroethoxy)phenyl]-5-methoxy-2-oxopyridin-1(2H)-yl}-4-(trifluoromethoxy)butanoyl]amino}-2-fluorobenzamide (Racemate)

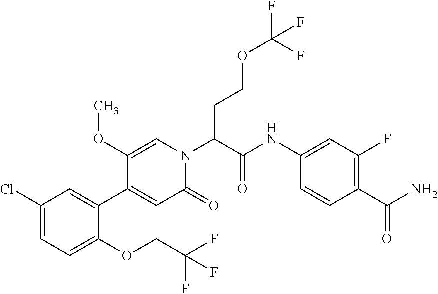 [SMILES: COc1cn(C(CCOC(F)(F)F)C(=O)Nc2ccc(C(N)=O)c(F)c2)c(=O)cc1-c1cc(Cl)ccc1OCC(F)(F)F]

60 mg (0.21 mmol) of 2-{4-[5-chloro-2-(2,2,2-trifluoroethoxy)phenyl]-5-methoxy-2-oxopyridin-1(2H)-yl}-4-(trifluoromethoxy)butanoic acid (racemate) and 27.5 mg (0.18 mmol, 1.5 eq.) of 4-amino-2-fluorobenzamide were reacted according to General Method 5. Yield: 58 mg (76% of theory)

LC/MS [Method 1]: Rₜ=1.02 min; MS (ESIpos): m/z=640 (M+H)⁺,

¹H-NMR (400 MHz, DMSO-d₆): δ [ppm]=10.78 (s, 1H), 7.71-7.61 (m, 2H), 7.57-7.47 (m, 3H), 7.44-7.39 (m, 1H), 7.35-7.30 (m, 2H), 7.26 (d, 1H), 6.35 (s, 1H), 5.78-5.71 (m, 1H), 4.78-4.69 (m, 2H), 4.17-4.09 (m, 1H), 3.97-3.89 (m, 1H), 3.60 (s, 3H), 2.69-2.55 (m, 2H).

### Example 43

### 4-{[2-{4-[5-Chloro-2-(2,2,2-trifluoroethoxy)phenyl]-5-methoxy-2-oxopyridin-1(2H)-yl}-3-(1-methyl-1H-pyrazol-3-yl)propanoyl]amino}benzoic acid (enantiomer 2)

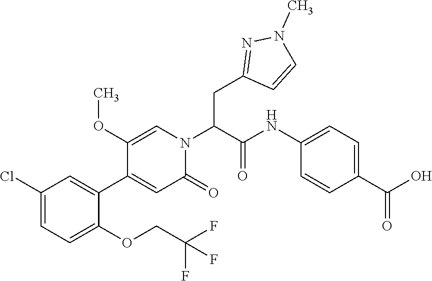 [SMILES: COc1cn(C(Cc2ccn(C)n2)C(=O)Nc2ccc(C(=O)O)cc2)c(=O)cc1-c1cc(Cl)ccc1OCC(F)(F)F]

Enantiomer separation of 90 mg of 4-{[2-{4-[5-chloro-2-(2,2,2-trifluoroethoxy)phenyl]-5-methoxy-2-oxopyridin-1(2H)-yl}-3-(1-methyl-1H-pyrazol-3-yl)propanoyl]amino}benzoic acid (racemate) gave 33 mg of enantiomer 1 (chiral HPLC: Rₜ=1.75 min) and 33 mg of the Example 43 title compound (enantiomer 2): chiral HPLC: Rₜ=3.55 min; 100% ee, 95% purity.

Separation method: column: Chiralpak AD-H 5 m 250 mm×20 mm; eluent: 70% carbon dioxide/30% ethanol; temperature: 40° C.; flow rate: 80 ml/min; pressure: 90 bar; UV detection: 210 nm.

Analysis: column: Daicel AD-H 5 μm 250 mm×4.6 mm; eluent: 75% carbon dioxide, 25% ethanol; flow rate: 3 ml/min; UV detection: 210 nm.

### Example 44

### 4-{[2-{4-[5-Chloro-2-(2-fluoroethoxy)phenyl]-5-methoxy-2-oxopyridin-1(2H)-yl}-3-(1,4-dioxan-2-yl)propanoyl]amino}benzoic acid (Diastereomer Mixture)

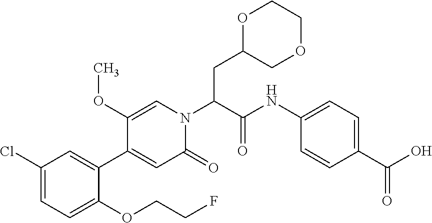 [SMILES: COc1cn(C(CC2COCCO2)C(=O)Nc2ccc(C(=O)O)cc2)c(=O)cc1-c1cc(Cl)ccc1OCCF]

70 mg (119 μmol) of methyl 4-{[2-{4-[5-chloro-2-(2-fluoroethoxy)phenyl]-5-methoxy-2-oxopyridin-1(2H)-yl}-3-(1,4-dioxan-2-yl)propanoyl]amino}benzoate (diastereomer mixture) were reacted according to General Method 2. Yield: 52 mg (76% of theory)

LC/MS [Method 1]: Rₜ=0.90/0.91 min; MS (ESIpos): m/z=575/575 (M+H)⁺,

¹H-NMR (400 MHz, DMSO-d₆): δ [ppm]=12.75 (bs, 1H), 10.72 and 10.69 (2×s, 1H), 7.96-7.85 (m, 2H), 7.79-7.72 (m, 2H), 7.46-7.41 (m, 1H), 7.38-7.32 (m, 1H), 7.30-7.26 (m, 1H), 7.18-7.14 (m, 1H), 6.34 and 6.32 (2×s, 1H), 5.82-5.67 (m, 1H), 4.70-4.53 (m, 2H), 4.32-4.19 (m, 2H), 3.78-3.65 (m, 2H), 3.65-3.58 (m, 4H), 3.57-3.39 (m, 3H), 3.39-3.19 (m, 2H), 2.35-2.20 (m, 1H), 2.20-2.04 (m, 1H).

### Example 45

### 5-{[2-{4-[5-Chloro-2-(2-fluoroethoxy)phenyl]-5-methoxy-2-oxopyridin-1(2H)-yl}-3-(1,4-dioxan-2-yl)propanoyl]amino}pyridine-2-carboxamide (Diastereomer Mixture)

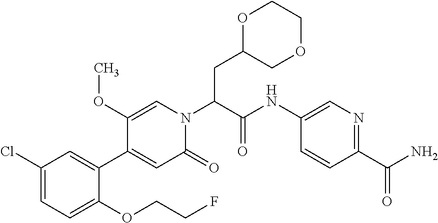 [SMILES: COc1cn(C(CC2COCCO2)C(=O)Nc2ccc(C(N)=O)nc2)c(=O)cc1-c1cc(Cl)ccc1OCCF]

50 mg (92% purity, 101 μmol) of 2-{4-[5-chloro-2-(2-fluoroethoxy)phenyl]-5-methoxy-2-oxopyridin-1(2H)-yl}-3-(1,4-dioxan-2-yl)propanoic acid (diastereomer mixture) and 21.4 mg (151 mol, 1.5 eq.) of 5-aminopyridine-2-carboxamide were reacted according to General Method 5. Yield: 49 mg (84% of theory)

LC/MS [Method 10]: Rₜ=1.47/1.50 min; MS (ESIpos): m/z=575/575 (M+H)⁺,

¹H-NMR (400 MHz, DMSO-d₆): δ [ppm]=10.87 and 10.84 (2×s, 1H), 8.87-8.83 (m, 1H), 8.26-8.19 (m, 1H), 8.04-7.98 (m, 2H), 7.55-7.49 (m, 1H), 7.46-7.41 (m, 1H), 7.38-7.32 (m, 1H), 7.30-7.26 (m, 1H), 7.19-7.14 (m, 1H), 6.35 and 6.33 (2×s, 1H), 5.81-5.66 (m, 1H), 4.71-4.53 (m, 2H), 4.33-4.19 (m, 2H), 3.79-3.57 (m, 6H), 3.56-3.34 (m, 3H), 3.28-3.19 (m, 1H), 2.38-2.24 (m, 1H), 2.22-2.05 (m, 1H).

### Example 46

### 5-{[2-{4-[5-Chloro-2-(2-fluoroethoxy)phenyl]-5-methoxy-2-oxopyridin-1(2H)-yl}-3-(1,4-dioxan-2-yl)propanoyl]amino}-N-methylpyridine-2-carboxamide (Diastereomer Mixture)

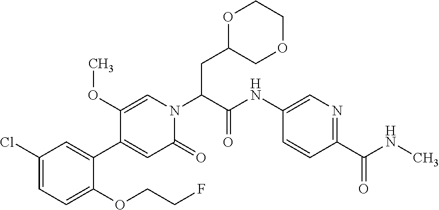 [SMILES: CNC(=O)c1ccc(NC(=O)C(CC2COCCO2)n2cc(OC)c(-c3cc(Cl)ccc3OCCF)cc2=O)cn1]

50 mg (92% purity, 101 μmol) of 2-{4-[5-chloro-2-(2-fluoroethoxy)phenyl]-5-methoxy-2-oxopyridin-1(2H)-yl}-3-(1,4-dioxan-2-yl)propanoic acid (diastereomer mixture) and 23.3 mg (151 mol, 1.5 eq.) of 5-amino-N-methylpyridine-2-carboxamide were reacted according to General Method 5. Yield: 53 mg (89% of theory)

LC/MS [Method 1]: Rₜ=0.84/0.86 min; MS (ESIpos): m/z=589/589 (M+H)⁺,

¹H-NMR (400 MHz, DMSO-d₆): δ [ppm]=10.85 and 10.82 (2×s, 1H), 8.91-8.83 (m, 1H), 8.69-8.61 (m, 1H), 8.25-8.17 (m, 1H), 8.03-7.97 (m, 1H), 7.47-7.40 (m, 1H), 7.38-7.31 (m, 1H), 7.30-7.25 (m, 1H), 7.19-7.13 (m, 1H), 6.35 and 6.33 (2×s, 1H), 5.79-5.66 (m, 1H), 4.71-4.53 (m, 2H), 4.32-4.19 (m, 2H), 3.79-3.58 (m, 6H), 3.58-3.34 (m, 3H), 3.28-3.20 (m, 1H), 2.80 (d, 3H), 2.38-2.24 (m, 1H), 2.23-2.04 (m, 1H).

### Example 47

### 4-{[2-{4-[5-Chloro-2-(2-fluoroethoxy)phenyl]-5-methoxy-2-oxopyridin-1(2H)-yl}-3-(1,4-dioxan-2-yl)propanoyl]amino}-2-fluorobenzamide (Diastereomer Mixture)

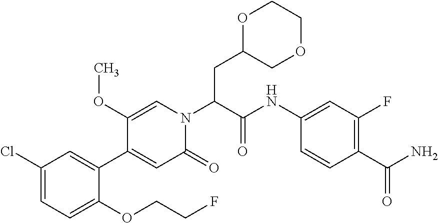 [SMILES: COc1cn(C(CC2COCCO2)C(=O)Nc2ccc(C(N)=O)c(F)c2)c(=O)cc1-c1cc(Cl)ccc1OCCF]

50 mg (92% purity, 101 μmol) of 2-{4-[5-chloro-2-(2-fluoroethoxy)phenyl]-5-methoxy-2-oxopyridin-1(2H)-yl}-3-(1,4-dioxan-2-yl)propanoic acid (diastereomer mixture) and 24.1 mg (151 μmol, 1.5 eq.) of 4-amino-2-fluorobenzamide were reacted according to General Method 5. Yield: 48 mg (80% of theory).

LC/MS [Method 2]: Rₜ=2.53/2.59 min; MS (ESIpos): m/z=592/592 (M+H)⁺,

¹H-NMR (400 MHz, DMSO-d₆): δ [ppm]=10.79 and 10.75 (2×s, 1H), 7.72-7.61 (m, 2H), 7.58-7.47 (m, 2H), 7.47-7.40 (m, 2H), 7.37-7.31 (m, 1H), 7.30-7.26 (m, 1H), 7.19-7.14 (m, 1H), 6.34 and 6.32 (2×s, 1H), 5.77-5.65 (m, 1H), 4.70-4.53 (m, 2H), 4.32-4.20 (m, 2H), 3.78-3.56 (m, 6H), 3.55-3.33 (m, 3H), 3.28-3.18 (m, 1H), 2.36-2.21 (m, 1H), 2.19-2.03 (m, 1H).

### Example 48

### 2-{4-[5-Chloro-2-(2-fluoroethoxy)phenyl]-5-methoxy-2-oxopyridin-1(2H)-yl}-3-(1,4-dioxan-2-yl)-N-(2-methyl-2H-indazol-5-yl)propanamide (Diastereomer Mixture)

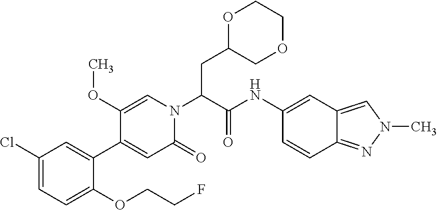 [SMILES: COc1cn(C(CC2COCCO2)C(=O)Nc2ccc3nn(C)cc3c2)c(=O)cc1-c1cc(Cl)ccc1OCCF]

50 mg (92% purity, 101 μmol) of 2-{4-[5-chloro-2-(2-fluoroethoxy)phenyl]-5-methoxy-2-oxopyridin-1(2H)-yl}-3-(1,4-dioxan-2-yl)propanoic acid (diastereomer mixture) and 22.3 mg (151 μmol, 1.5 eq.) of 2-methyl-2H-indazole-5-amine were reacted according to General Method 5. Yield: 28 mg (48% of theory).

LC/MS [Method 10]: Rₜ=1.58/1.61 min; MS (ESIpos): m/z=585/585 (M+H)⁺,

¹H-NMR (400 MHz, DMSO-d₆): δ [ppm]=10.39 and 10.35 (2×s, 1H), 8.28-8.23 (m, 1H), 8.15-8.09 (m, 1H), 7.57-7.51 (m, 1H), 7.46-7.36 (m, 2H), 7.34-7.26 (m, 2H), 7.19-7.14 (m, 1H), 6.35 and 6.32 (2×s, 1H), 5.84-5.73 (m, 1H), 4.70-4.53 (m, 2H), 4.32-4.20 (m, 2H), 4.13 and 4.13 (2×s, 3H), 3.81-3.56 (m, 6H), 3.56-3.34 (m, 3H), 3.29-3.18 (m, 1H), 2.35-2.19 (m, 1H), 2.19-2.00 (m, 1H).

### Example 49

### 4-[(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]benzoic acid (Diastereomer Mixture)

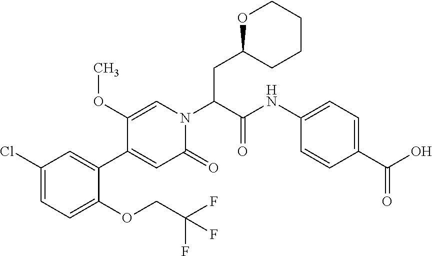 [SMILES: COc1cn(C(C[C@@H]2CCCCO2)C(=O)Nc2ccc(C(=O)O)cc2)c(=O)cc1-c1cc(Cl)ccc1OCC(F)(F)F]

135 mg (217 μmol) of methyl 4-[(2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]benzoate (diastereomer mixture) were reacted according to General Method 2. Yield: 108 mg (82% of theory).

LC/MS [Method 1]: Rₜ=1.04 min; MS (ESIpos): m/z=609 (M+H)⁺,

¹H-NMR (400 MHz, DMSO-d₆): δ [ppm]=12.74 (bs, 1H), 10.71 and 10.61 (2×s, 1H), 7.94-7.86 (m, 2H), 7.79-7.72 (m, 2H), 7.52-7.46 (m, 1H), 7.38-7.29 (m, 2H), 7.28-7.22 (m, 1H), 6.33 and 6.30 (2×s, 1H), 5.84-5.67 (m, 1H), 4.83-4.67 (m, 2H), 3.90-3.78 (m, 1H), 3.60 and 3.60 (2×s, 3H), 3.27-2.98 (m, 3H), 2.41-2.12 (m, 2H), 1.80-1.70 (m, 1H), 1.68-1.50 (m, 1H), 1.49-1.14 (m, 4H).

### Example 50

### 4-[(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]benzoic acid (diastereomer 2)

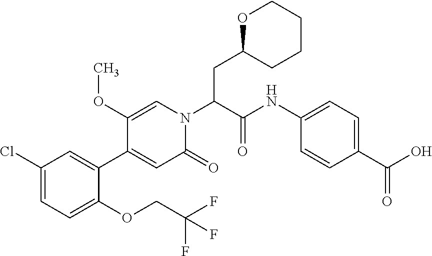 [SMILES: COc1cn(C(C[C@@H]2CCCCO2)C(=O)Nc2ccc(C(=O)O)cc2)c(=O)cc1-c1cc(Cl)ccc1OCC(F)(F)F]

Enantiomer separation of 108 mg of 4-[(2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]benzoic acid (diastereomer mixture) gave 52 mg of diastereomer 1 (chiral HPLC: Rₜ=4.0 min) and 44 mg of the Example 50 title compound (diastereomer 2): chiral HPLC: Rₜ=7.9 min; 100% ee.

Separation method: column: Daicel Chiralpak AZ-H 5 μm, 250 mm×20 mm; eluent: 75% carbon dioxide/25% ethanol; temperature: 40° C.; flow rate: 80 ml/min; pressure: 90 bar; UV detection: 210 nm.

Analysis: column: Daicel AZ-H 5 μm, 250 mm×4.6 mm; eluent: 70% carbon dioxide, 30% ethanol; flow rate: 3 ml/min; UV detection: 210 nm.

### Example 51

### 2-{4-[5-Chloro-2-(2,2,2-trifluoroethoxy)phenyl]-5-methoxy-2-oxopyridin-1(2H)-yl}-N-(2-methyl-2H-indazol-5-yl)-3-[(2S)-tetrahydro-2H-pyran-2-yl]propanamide (Diastereomer Mixture)

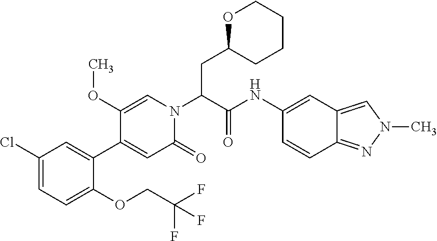 [SMILES: COc1cn(C(C[C@@H]2CCCCO2)C(=O)Nc2ccc3nn(C)cc3c2)c(=O)cc1-c1cc(Cl)ccc1OCC(F)(F)F]

40 mg (85% purity, 69 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 15.6 mg (104 μmol, 1.5 eq.) of 2-methyl-2H-indazole-5-amine were reacted according to General Method 5. Yield: 34 mg (79% of theory).

LC/MS [Method 1]: Rₜ=1.03 min; MS (ESIpos): m/z=619 (M+H)⁺,

¹H-NMR (400 MHz, DMSO-d₆): δ [ppm]=10.36 and 10.27 (2×s, 1H), 8.27-8.22 (m, 1H), 8.14-8.09 (m, 1H), 7.57-7.47 (m, 2H), 7.42-7.29 (m, 3H), 7.28-7.23 (m, 1H), 6.33 and 6.31 (2×s, 1H), 5.85-5.71 (m, 1H), 4.83-4.68 (m, 2H), 4.13 and 4.12 (2×s, 3H), 3.92-3.80 (m, 1H), 3.60 and 3.60 (2×s, 3H), 3.26-3.00 (m, 2H), 2.38-2.08 (m, 2H), 1.80-1.71 (m, 1H), 1.68-1.52 (m, 1H), 1.48-1.14 (m, 4H).

### Example 52

### 5-[(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]-N-methylpyridine-2-carboxamide (Diastereomer Mixture)

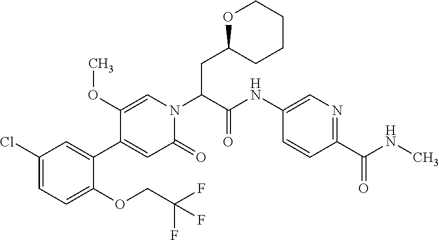 [SMILES: CNC(=O)c1ccc(NC(=O)C(C[C@@H]2CCCCO2)n2cc(OC)c(-c3cc(Cl)ccc3OCC(F)(F)F)cc2=O)cn1]

40 mg (85% purity, 69 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 16.1 mg (104 μmol, 1.5 eq.) of 5-amino-N-methylpyridine-2-carboxamide were reacted according to General Method 5. Yield: 34 mg (79% of theory).

LC/MS [Method 1]: Rₜ=1.02 min; MS (ESIpos): m/z=623 (M+H)⁺,

¹H-NMR (400 MHz, DMSO-d₆): δ [ppm]=10.84 and 10.74 (2×s, 1H), 8.89-8.86 (m, 1H), 8.68-8.62 (m, 1H), 8.25-8.18 (m, 1H), 8.02-7.97 (m, 1H), 7.52-7.47 (m, 1H), 7.36-7.29 (m, 2H), 7.28-7.23 (m, 1H), 6.33 and 6.31 (2×s, 1H), 5.82-5.64 (m, 1H), 4.81-4.69 (m, 2H), 3.90-3.77 (m, 1H), 3.60 and 3.60 (2×s, 3H), 3.26-2.97 (m, 2H), 2.80 (d, 3H), 2.43-2.13 (m, 2H), 1.80-1.71 (m, 1H), 1.67-1.52 (m, 1H), 1.48-1.14 (m, 4H).

### Example 53

### 4-[(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]-2-fluorobenzamide (Diastereomer Mixture)

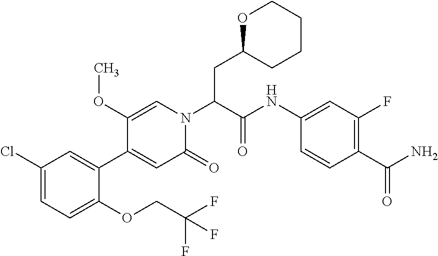 [SMILES: COc1cn(C(C[C@@H]2CCCCO2)C(=O)Nc2ccc(C(N)=O)c(F)c2)c(=O)cc1-c1cc(Cl)ccc1OCC(F)(F)F]

40 mg (85% purity, 69 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 16.5 mg (104 μmol, 1.5 eq.) of 4-amino-2-fluorobenzamide were reacted according to General Method 5. Yield: 33 mg (76% of theory).

LC/MS [Method 1]: Rₜ=1.01 min; MS (ESIpos): m/z=626/626 (M+H)⁺,

¹H-NMR (400 MHz, DMSO-d₆): δ [ppm]=10.77 and 10.67 (2×s, 1H), 7.72-7.60 (m, 2H), 7.58-7.39 (m, 4H), 7.37-7.20 (m, 3H), 6.33 and 6.31 (2×s, 1H), 5.80-5.62 (m, 1H), 4.83-4.67 (m, 2H), 3.90-3.76 (m, 1H), 3.60 (s, 3H), 3.23-2.97 (m, 2H), 2.41-2.10 (m, 2H), 1.80-1.69 (m, 1H), 1.67-1.50 (m, 1H), 1.48-1.14 (m, 4H).

### Example 54

### 5-[(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl)amino]-N-methylpyridine-2-carboxamide (Diastereomer Mixture)

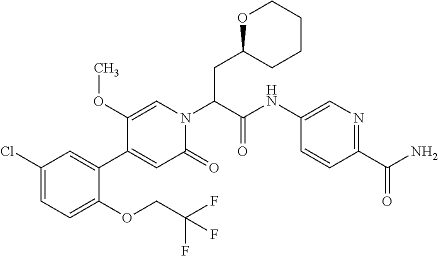 [SMILES: COc1cn(C(C[C@@H]2CCCCO2)C(=O)Nc2ccc(C(N)=O)nc2)c(=O)cc1-c1cc(Cl)ccc1OCC(F)(F)F]

40 mg (85% purity, 69 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 14.7 mg (104 μmol, 1.5 eq.) of 5-aminopyridine-2-carboxamide were reacted according to General Method 5. Yield: 32 mg (76% of theory).

LC/MS [Method 1]: Rₜ=0.98 min; MS (ESIpos): m/z=609 (M+H)⁺,

¹H-NMR (400 MHz, DMSO-d₆): δ [ppm]=10.86 and 10.75 (2×s, 1H), 8.89-8.82 (m, 1H), 8.27-8.17 (m, 1H), 8.06-7.96 (m, 2H), 7.56-7.47 (m, 2H), 7.38-7.29 (m, 2H), 7.29-7.21 (m, 1H), 6.33 and 6.31 (2×s, 1H), 5.83-5.65 (m, 1H), 4.83-4.68 (m, 2H), 3.90-3.79 (m, 1H), 3.60 and 3.60 (2×s, 3H), 3.27-2.98 (m, 2H), 2.43-2.12 (m, 2H), 1.80-1.71 (m, 1H), 1.67-1.51 (m, 1H), 1.48-1.14 (m, 4H).

### Example 55

### 4-[(2-{4-[5-Chloro-2-(2,2,2-trifluoroethoxy)phenyl]-5-methoxy-2-oxopyridin-1(2H)-yl}-4-[(1-methylcyclobutyl)oxy]butanoyl)amino]benzoic acid (Racemate)

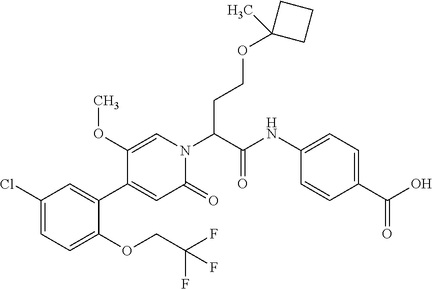 [SMILES: COc1cn(C(CCOC2(C)CCC2)C(=O)Nc2ccc(C(=O)O)cc2)c(=O)cc1-c1cc(Cl)ccc1OCC(F)(F)F]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 33 mg (0.24 mmol, 1.0 eq.) of 4-aminobenzoic acid were reacted according to General Method 5. Yield: 116 mg (78% of theory).

LC/MS [Method 10]: Rₜ=2.06 min; MS (ESIpos): m/z=623 (M+H)⁺,

¹H-NMR (400 MHz, DMSO-d₆): δ [ppm]=12.73 (br. s., 1H), 10.73 (s, 1H), 7.93-7.87 (m, 1H), 7.79-7.74 (m, 1H), 7.49 (dd, 1H), 7.36-7.24 (m, 3H), 6.34 (s, 1H), 5.83-5.76 (m, 1H), 4.83-4.67 (m, 2H), 3.60 (s, 3H), 3.19-3.10 (m, 1H), 2.41-2.31 (m, 2H), 2.04-1.84 (m, 2H), 1.72-1.61 (m, 2H), 1.61-1.41 (m, 2H), 1.17 (s, 3H).

### Example 56

### 5-[[(4S)-2-[4-[5-Chloro-2-(2,2,2-trifluoroethoxy)phenyl]-5-methoxy-2-oxo-1-pyridyl]-4-methoxypentanoyl]amino]pyridine-2-carboxamide (Diastereomer Mixture)

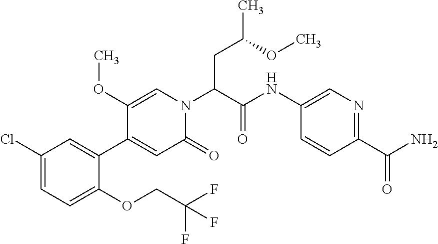 [SMILES: COc1cn(C(C[C@H](C)OC)C(=O)Nc2ccc(C(N)=O)nc2)c(=O)cc1-c1cc(Cl)ccc1OCC(F)(F)F]

100 mg (0.22 mmol) of (4S)-2-[4-[5-chloro-2-(2,2,2-trifluoroethoxy)phenyl]-5-methoxy-2-oxo-1-pyridyl]-4-methoxypentanoic acid (racemate) were initially charged in 2.5 ml of pyridine and the mixture was heated to 50° C. Subsequently, 0.10 ml of propylphosphonic anhydride (50% in ethyl acetate, 4.0 eq.) was added and the mixture was stirred for a further 10 min. Subsequently, 32 mg (0.24 mmol, 1.1 eq.) of 5-aminopyridine-2-carboxamide were added and the mixture was stirred at 50° C. overnight. Thereafter, a further 2.0 eq. of propylphosphonic anhydride and, after 30 min, another 1.0 eq. of 5-aminopyridine-2-carboxamide was added. The mixture was stirred for a further 30 min and then brought to RT. The mixture was diluted with 2 ml of acetonitrile and separated by means of preparative HPLC (water/acetonitrile gradient with 0.1% formic acid). Yield: 5 mg (93% purity, 3% of theory).

LC/MS [Method 1]: Rₜ=0.96 min; MS (ESIpos): m/z=583 (M+H)⁺,

¹H-NMR (400 MHz, DMSO-d₆): δ [ppm]=10.94-10.82 (m, 1H), 8.86 (d, 1H), 8.26-8.21 (m, 1H), 8.04-7.98 (m, 2H), 7.55-7.21 (m, 5H), 6.34 (d, 1H), 5.86-5.76 (m, 1H), 4.80-4.70 (m, 2H), 3.61 (s, 3H), 3.20-3.03 (m, 4H), 2.42-2.31 (m, 1H), 2.27-2.17 (m, 1H), 1.17-1.10 (m, 3H).

### Example 57

### 5-[[2-[4-[5-Chloro-2-(2,2,2-trifluoroethoxy)phenyl]-5-methoxy-2-oxo-1-pyridyl]-4-(1-methylcyclobutoxy)butanoyl]amino]-N-methylpyridine-2-carboxamide (Racemate)

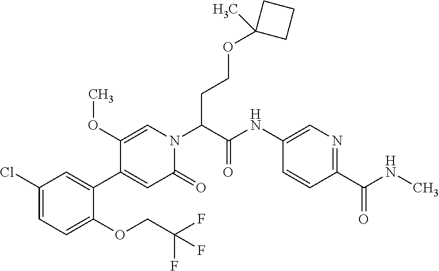 [SMILES: CNC(=O)c1ccc(NC(=O)C(CCOC2(C)CCC2)n2cc(OC)c(-c3cc(Cl)ccc3OCC(F)(F)F)cc2=O)cn1]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 36 mg (0.24 mmol, 1.0 eq.) of 5-amino-N-methylpyridine-2-carboxamide were reacted according to General Method 5. Yield: 123 mg (81% of theory).

LC/MS [Method 10]: Rₜ=2.03 min; MS (ESIpos): m/z=637 (M+H)⁺,

¹H-NMR (400 MHz, DMSO-d₆): δ [ppm]=10.87 (s, 1H), 8.89 (d, 1H), 8.69-8.62 (m, 1H), 8.25-8.19 (m, 1H), 8.00 (d, 1H), 7.50 (dd, 1H), 7.36-7.23 (m, 3H), 6.34 (s, 1H), 5.83-5.75 (m, 1H), 4.83-4.66 (m, 2H), 3.61 (s, 3H), 3.40-3.30 (m, 1H, partly concealed), 3.19-3.10 (m, 1H), 2.80 (d, 3H), 2.45-2.35 (m, 2H), 2.03-1.84 (m, 2H), 1.73-1.61 (m, 2H), 1.60-1.42 (m, 2H), 1.17 (s, 3H).

### Example 58

### 2-[4-[5-Chloro-2-(2,2,2-trifluoroethoxy)phenyl]-5-methoxy-2-oxo-1-pyridyl]-4-(1-methylcyclobutoxy)-N-(2-methylindazol-5-yl)butanamide (Racemate)

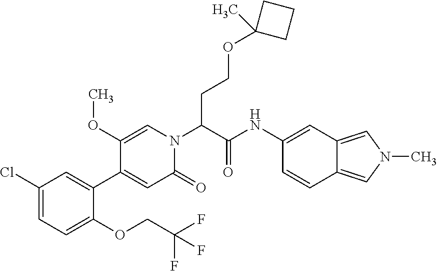 [SMILES: COc1cn(C(CCOC2(C)CCC2)C(=O)Nc2ccc3cn(C)cc3c2)c(=O)cc1-c1cc(Cl)ccc1OCC(F)(F)F]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 35 mg (0.24 mmol, 1.0 eq.) of 2-methyl-2H-indazole-5-amine were reacted according to General Method 5. Yield: 130 mg (86% of theory).

LC/MS [Method 10]: Rₜ=2.06 min; MS (ESIpos): m/z=633 (M+H)⁺,

¹H-NMR (400 MHz, DMSO-d₆): δ [ppm]=10.36 (s, 1H), 8.25 (s, 1H), 8.16-8.12 (m, 1H), 7.57-7.52 (m, 1H), 7.51-7.47 (m, 1H), 7.39-7.36 (m, 1H), 7.35-7.23 (m, 3H), 6.34 (s, 1H), 5.85-5.78 (m, 1H), 4.84-4.67 (m, 2H), 4.13 (s, 3H), 3.61 (s, 3H), 3.21-3.12 (m, 1H), 2.40-2.31 (m, 2H), 2.05-1.87 (m, 2H), 1.72-1.62 (m, 2H), 1.61-1.43 (m, 2H), 1.18 (s, 3H).

### Example 59

### 4-[[2-[4-[5-Chloro-2-(2,2,2-trifluoroethoxy)phenyl]-5-methoxy-2-oxo-1-pyridyl]-4-(1-methylcyclobutoxy)butanoyl]amino]-2-fluorobenzamide (Racemate)

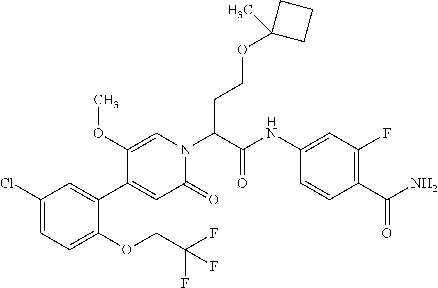 [SMILES: COc1cn(C(CCOC2(C)CCC2)C(=O)Nc2ccc(C(N)=O)c(F)c2)c(=O)cc1-c1cc(Cl)ccc1OCC(F)(F)F]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 37 mg (0.24 mmol, 1.0 eq.) of 4-amino-2-fluorobenzamide were reacted according to General Method 5. Yield: 125 mg (82% of theory).

LC/MS [Method 10]: Rₜ=2.01 min; MS (ESIpos): m/z=640 (M+H)⁺,

¹H-NMR (400 MHz, DMSO-d₆): δ [ppm]=10.79 (s, 1H), 7.72-7.41 (m, 6H), 7.34-7.24 (m, 3H), 6.34 (s, 1H), 5.80-5.73 (m, 1H), 4.84-4.66 (m, 2H), 3.60 (s, 3H), 3.37-3.30 (m, 1H, partly concealed), 3.18-3.10 (m, 1H), 2.44-2.31 (m, 2H), 2.04-1.84 (m, 2H), 1.73-1.61 (m, 2H), 1.60-1.43 (m, 2H), 1.17 (s, 3H).

### Example 60

### 5-[[2-[4-[5-Chloro-2-(2,2,2-trifluoroethoxy)phenyl]-5-methoxy-2-oxo-1-pyridyl]-4-(1-methylcyclobutoxy)butanoyl]amino]pyridine-2-carboxamide (Racemate)

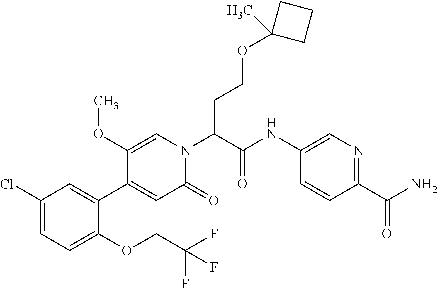 [SMILES: COc1cn(C(CCOC2(C)CCC2)C(=O)Nc2ccc(C(N)=O)nc2)c(=O)cc1-c1cc(Cl)ccc1OCC(F)(F)F]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 33 mg (0.24 mmol, 1.0 eq.) of 5-aminopyridine-2-carboxamide were reacted according to General Method 5. Yield: 123 mg (83% of theory).

LC/MS [Method 10]: Rₜ=1.95 min; MS (ESIpos): m/z=623 (M+H)⁺,

¹H-NMR (400 MHz, DMSO-d₆): δ [ppm]=10.89 (s, 1H), 8.87 (d, 1H), 8.24 (dd, 1H), 8.04-7.98 (m, 2H), 7.54-7.47 (m, 2H), 7.36-7.23 (m, 3H), 6.35 (s, 1H), 5.83-5.76 (m, 1H), 4.83-4.67 (m, 2H), 3.61 (s, 3H), 3.37-3.30 (m, 1H, partly concealed), 3.19-3.11 (m, 1H), 2.44-2.35 (m, 2H), 2.03-1.84 (m, 2H), 1.73-1.61 (m, 2H), 1.60-1.43 (m, 2H), 1.17 (s, 3H).

### Example 61

### 5-[[2-[4-[5-Chloro-2-(2,2,2-trifluoroethoxy)phenyl]-5-methoxy-2-oxo-1-pyridyl]-4-(1-methylcyclobutoxy)butanoyl]amino]-N-ethylpyridine-2-carboxamide (Racemate)

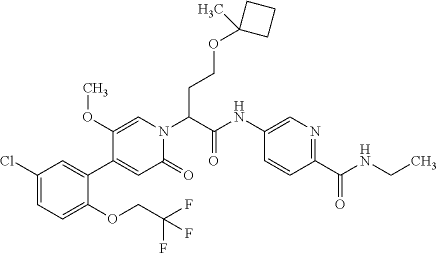 [SMILES: CCNC(=O)c1ccc(NC(=O)C(CCOC2(C)CCC2)n2cc(OC)c(-c3cc(Cl)ccc3OCC(F)(F)F)cc2=O)cn1]

120 mg (0.24 mmol) of 2-{4-[5-chloro-2-(2,2,2-trifluoroethoxy)phenyl]-5-methoxy-2-oxopyridin-1(2H)-yl}-4-[(1-methylcyclobutyl)oxy]butanoic acid (racemate) and 39 mg (0.24 mmol, 1.0 eq.) of 5-amino-N-ethylpyridine-2-carboxamide were reacted according to General Method 5. Yield: 130 mg (84% of theory).

LC/MS [Method 10]: Rₜ=2.12 min; MS (ESIpos): m/z=651 (M+H)⁺,

¹H-NMR (400 MHz, DMSO-d₆): δ [ppm]=10.88 (s, 1H), 8.89 (d, 1H), 8.72-8.66 (m, 1H), 8.23 (dd, 1H), 8.00 (d, 1H), 7.50 (dd, 1H), 7.35-7.24 (m, 3H), 6.34 (s, 1H), 5.83-5.75 (m, 1H), 4.84-4.67 (m, 2H), 3.61 (s, 3H), 3.40-3.25 (m, partly concealed), 3.19-3.10 (m, 1H), 2.45-2.36 (m, 2H), 2.03-1.85 (m, 2H), 1.73-1.61 (m, 2H), 1.60-1.43 (m, 2H), 1.17 (s, 3H), 1.11 (t, 3H).

### Example 62

### 4-{[2-{4-[5-Chloro-2-(2,2,2-trifluoroethoxy)phenyl]-5-methoxy-2-oxopyridin-1(2H)-yl}-3-(1,4-dioxan-2-yl)propanoyl]amino}benzoic acid (diastereomer 3)

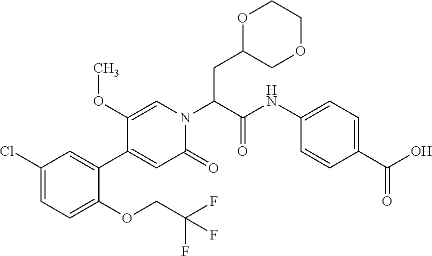 [SMILES: COc1cn(C(CC2COCCO2)C(=O)Nc2ccc(C(=O)O)cc2)c(=O)cc1-c1cc(Cl)ccc1OCC(F)(F)F]

Diastereomer separation and enantiomer separation of 216 mg of 4-{[2-{4-[5-chloro-2-(2,2,2-trifluoroethoxy)phenyl]-5-methoxy-2-oxopyridin-1(2H)-yl}-3-(1,4-dioxan-2-yl)propanoyl]amino}benzoic acid (diastereomer mixture) gave 30 mg of diastereomer 1 (chiral HPLC: Rₜ=6.1 min), 28 mg of diastereomer 2 (chiral HPLC: Rₜ=6.7 min), 18 mg of diastereomer 4 (chiral HPLC: Rₜ=21.5 min) and 45 mg of the Example 62 title compound (diastereomer 3): chiral HPLC: Rₜ=9.7 min; 100% ee. Diastereomer 3, after separation on a chiral column, was purified by means of preparative RP-HPLC (water/acetonitrile gradient). Yield: 22 mg.

Separation method: column: Daicel AD-H 5 μm, 250 mm×20 mm; eluent: 80% carbon dioxide/20% ethanol; temperature: 40° C.; flow rate: 80 ml/min; pressure: 100 bar; UV detection: 210 nm.

Analysis: column: Daicel AD-H 5 μm, 250 mm×4.6 mm; eluent: 80% carbon dioxide, 20% ethanol; flow rate: 3 ml/min; UV detection: 210 nm.

LC/MS [Method 1]: Rₜ=0.93 min; MS (ESIpos): m/z=611 (M+H)⁺,

¹H-NMR (400 MHz, DMSO-d₆): δ [ppm]=12.76 (bs, 1H), 10.71 (s, 1H), 7.94-7.88 (m, 2H), 7.78-7.73 (m, 2H), 7.49 (dd, 1H), 7.38-7.32 (m, 2H), 7.27 (d, 1H), 6.34 (s, 1H), 5.77 (dd, 1H), 4.76 (q, 2H), 3.78-3.37 (m, 10H), 3.30-3.24 (m, 2H), 2.28-2.19 (m, 1H), 2.17-2.07 (m, 1H).

### Example 63

### 5-{[2-{4-[5-Chloro-2-(2,2,2-trifluoroethoxy)phenyl]-5-methoxy-2-oxopyridin-1(2H)-yl}-3-(1,4-dioxan-2-yl)propanoyl]amino}-N-methylpyridine-2-carboxamide (diastereomer 3)

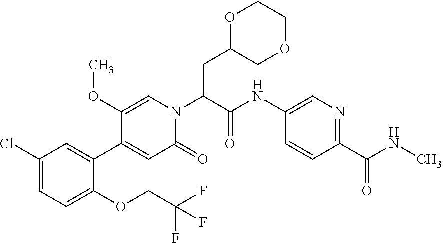 [SMILES: CNC(=O)c1ccc(NC(=O)C(CC2COCCO2)n2cc(OC)c(-c3cc(Cl)ccc3OCC(F)(F)F)cc2=O)cn1]

Diastereomer separation and enantiomer separation of 284 mg of 5-{[2-{4-[5-chloro-2-(2,2,2-trifluoroethoxy)phenyl]-5-methoxy-2-oxopyridin-1(2H)-yl-3-(1,4-dioxan-2-yl)propanoyl]amino}-N-methylpyridine-2-carboxamide (diastereomer mixture) gave 28 mg of the Example 63 title compound (diastereomer 3): chiral HPLC (second separation method): Rₜ=6.3 min; 100% ee.

By the first separation method diastereomer 4 (chiral HPLC: Rₜ=6.8 min) was separated from diastereomers 1 to 3, and by the second separation method diastereomer 1 (chiral HPLC: Rₜ=4.2 min), diastereomer 2 (chiral HPLC: Rₜ=5.2 min) and diastereomer 3 (chiral HPLC: Rₜ=6.3 min) were separated.

First separation method: column: Daicel Chiralpak OD-H 5 μm, 250 mm×20 mm; eluent: 80% carbon dioxide/20% methanol; temperature: 40° C.; flow rate: 80 ml/min; pressure: 90 bar; UV detection: 210 nm.

Second separation method: column: Daicel IA 5 μm, 250 mm×20 mm; eluent: 75% carbon dioxide/25% isopropanol; temperature: 40° C.; flow rate: 80 ml/min; pressure: 90 bar; UV detection: 210 nm.

Analysis: column: Daicel OD-H 5 μm, 250 mm×4.6 mm; eluent: 80% carbon dioxide, 20% methanol; flow rate: 3 ml/min; UV detection: 210 nm.

LC/MS [Method 1]: Rₜ=0.92 min; MS (ESIpos): m/z=625 (M+H)⁺,

¹H-NMR (400 MHz, DMSO-d₆): δ [ppm]=10.84 (s, 1H), 8.86 (d, 1H), 8.66 (q, 1H), 8.22 (dd, 1H), 8.01 (d, 1H), 7.49 (dd, 1H), 7.38-7.25 (m, 3H), 6.35 (s, 1H), 5.79-5.71 (m, 1H), 4.76 (q, 2H), 3.79-3.39 (m, 10H), 3.29-3.23 (m, 1H), 2.80 (d, 3H), 2.37-2.22 (m, 1H), 2.20-2.08 (m, 1H).

### Example 64

### N-(Quinoxalin-6-yl)-2-{4-[5-chloro-2-(2,2,2-trifluoroethoxy)phenyl]-5-methoxy-2-oxopyridin-1(2H)-yl}pentanamide (Racemate)

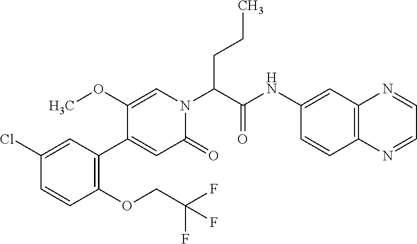 [SMILES: CCCC(C(=O)Nc1ccc2nccnc2c1)n1cc(OC)c(-c2cc(Cl)ccc2OCC(F)(F)F)cc1=O]

45.0 mg (101 μmol) of 2-{4-[5-chloro-2-(2,2,2-trifluoroethoxy)phenyl]-5-methoxy-2-oxopyridin-1(2H)-yl}pentanoic acid (racemate) and 21.9 mg (0.151 mmol) of quinoxaline-6-amine in 1.0 ml of pyridine were reacted according to General Method 5. Yield: 47.8 mg (85% of theory).

LC-MS (Method 10): Rₜ=1.98 min; MS (ESIpos): m/z=561 [M+H]⁺

¹H-NMR (400 MHz, DMSO-d₆): δ [ppm]=10.98 (s, 1H), 8.90 (d, 1H), 8.84 (d, 1H), 8.54 (d, 1H), 8.08 (d, 1H), 7.99 (dd, 1H), 7.50 (dd, 1H), 7.39 (s, 1H), 7.35 (d, 1H), 7.27 (d, 1H), 6.35 (s, 1H), 5.78 (dd, 1H), 4.76 (q, 2H), 3.63 (s, 3H), 2.24-2.09 (m, 2H), 1.38-1.22 (m, 2H), 0.95 (t, 3H).

### Example 65

### 2-{4-[5-Chloro-2-(2,2,2-trifluoroethoxy)phenyl]-5-methoxy-2-oxopyridin-1(2H)-yl}-N-(2-methyl-2H-indazol-5-yl)pentanamide (Racemate)

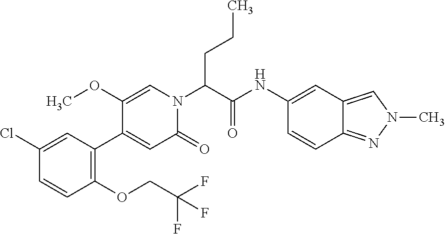 [SMILES: CCCC(C(=O)Nc1ccc2nn(C)cc2c1)n1cc(OC)c(-c2cc(Cl)ccc2OCC(F)(F)F)cc1=O]

45.0 mg (101 μmol) of 2-{4-[5-chloro-2-(2,2,2-trifluoroethoxy)phenyl]-5-methoxy-2-oxopyridin-1(2H)-yl}pentanoic acid (racemate) and 22.2 mg (0.151 mmol) of 2-methyl-2H-indazole-5-amine in 1.0 ml of pyridine were reacted according to General Method 5. Yield: 39.8 mg (70% of theory).

LC-MS (Method 10): Rₜ=1.91 min; MS (ESIpos): m/z=563 [M+H]⁺

¹H-NMR (400 MHz, DMSO-d₆): δ [ppm]=10.40 (s, 1H), 8.25 (s, 1H), 8.14 (d, 1H), 7.55 (d, 1H), 7.49 (dd, 1H), 7.39 (s, 1H), 7.34 (d, 1H), 7.31-7.24 (m, 2H), 6.33 (s, 1H), 5.75 (t, 1H), 4.76 (q, 2H), 4.13 (s, 3H), 3.61 (s, 3H), 2.08 (q, 2H), 1.35-1.20 (m, 2H), 0.93 (t, 3H).

### Example 66

### 5-{[2-{4-[5-Chloro-2-(2,2,2-trifluoroethoxy)phenyl]-5-methoxy-2-oxopyridin-1(2H)-yl}pentanoyl]amino}-N-methylpyridine-2-carboxamide (Racemate)

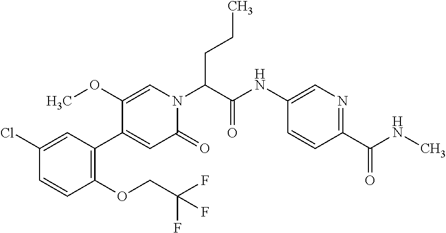 [SMILES: CCCC(C(=O)Nc1ccc(C(=O)NC)nc1)n1cc(OC)c(-c2cc(Cl)ccc2OCC(F)(F)F)cc1=O]

45.0 mg (101 μmol) of 2-{4-[5-chloro-2-(2,2,2-trifluoroethoxy)phenyl]-5-methoxy-2-oxopyridin-1(2H)-yl}pentanoic acid (racemate) and 23.3 mg (0.151 mmol) of 5-amino-N-methylpyridine-2-carboxamide in 1.0 ml of pyridine were reacted according to General Method 5. Yield: 39.6 mg (69% of theory).

LC-MS (Method 10): Rₜ=1.88 min; MS (ESIpos): m/z=567 [M+H]⁺

¹H-NMR (400 MHz, DMSO-d₆): δ [ppm]=10.91 (s, 1H), 8.86 (d, 1H), 8.66 (q, 1H), 8.21 (dd, 1H), 8.01 (d, 1H), 7.49 (dd, 1H), 7.34 (s, 1H), 7.34 (d, 1H), 7.27 (d, 1H), 6.34 (s, 1H), 5.71 (dd, 1H), 4.76 (q, 2H), 3.60 (s, 3H), 2.80 (d, 3H), 2.21-2.06 (m, 2H), 1.35-1.19 (m, 2H), 0.93 (t, 3H).

### Example 67

### 4-{[2-{4-[5-Chloro-2-(2,2,2-trifluoroethoxy)phenyl]-5-methoxy-2-oxopyridin-1(2H)-yl}pentanoyl]amino}benzoic acid (Racemate)

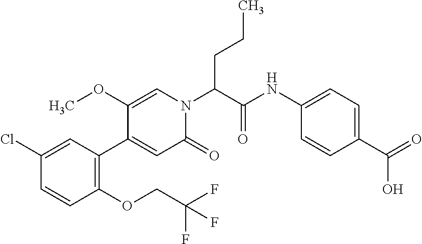 [SMILES: CCCC(C(=O)Nc1ccc(C(=O)O)cc1)n1cc(OC)c(-c2cc(Cl)ccc2OCC(F)(F)F)cc1=O]

45.0 mg (101 μmol) of 2-{4-[5-chloro-2-(2,2,2-trifluoroethoxy)phenyl]-5-methoxy-2-oxopyridin-1(2H)-yl}pentanoic acid (racemate) and 14.5 mg (106 μmol) of 4-aminobenzoic acid in 0.5 ml of pyridine were reacted according to General Method 5. Yield: 41.9 mg (75% of theory).

LC-MS (Method 10): Rₜ=1.92 min; MS (ESIpos): m/z=553 [M+H]⁺

¹H-NMR (400 MHz, DMSO-d₆): δ [ppm]=12.75 (br s, 1H), 10.76 (s, 1H), 7.93-7.89 (m, 2H), 7.77-7.72 (m, 2H), 7.49 (dd, 1H), 7.35-7.33 (m, 2H), 7.26 (d, 1H), 6.33 (s, 1H), 5.73 (dd, 1H), 4.76 (q, 2H), 3.60 (s, 3H), 2.17-2.02 (m, 2H), 1.33-1.18 (m, 2H), 0.92 (t, 3H).

### Example 68

### 5-{[2-{4-[5-Chloro-2-(2,2,2-trifluoroethoxy)phenyl]-5-methoxy-2-oxopyridin-1(2H)-yl}pentanoyl]amino}pyridine-2-carboxamide (Racemate)

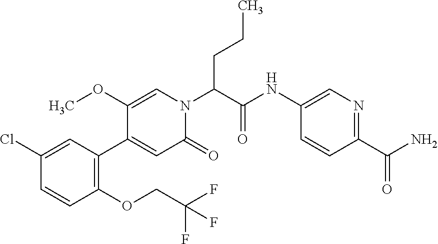 [SMILES: CCCC(C(=O)Nc1ccc(C(N)=O)nc1)n1cc(OC)c(-c2cc(Cl)ccc2OCC(F)(F)F)cc1=O]

45.0 mg (101 μmol) of 2-{4-[5-chloro-2-(2,2,2-trifluoroethoxy)phenyl]-5-methoxy-2-oxopyridin-1(2H)-yl}pentanoic acid (racemate) and 21.8 mg (95% purity, 151 μmol) of 5-aminopyridine-2-carboxamide in 1.0 ml of pyridine were reacted according to General Method 5. Yield: 49.2 mg (88% of theory).

LC-MS (Method 10): Rₜ=1.80 min; MS (ESIpos): m/z=553 [M+H]⁺

¹H-NMR (400 MHz, DMSO-d₆): δ [ppm]=10.92 (s, 1H), 8.84 (d, 1H), 8.22 (dd, 1H), 8.04-7.99 (m, 2H), 7.52 (br s, 1H), 7.49 (dd, 1H), 7.34 (s, 1H), 7.35 (d, 1H), 7.27 (d, 1H), 6.34 (s, 1H), 5.72 (dd, 1H), 4.76 (q, 2H), 3.61 (s, 3H), 2.20-2.06 (m, 2H), 1.35-1.19 (m, 2H), 0.93 (t, 3H).

### Example 69

### 5-{[4-tert-Butoxy-2-{4-[5-chloro-2-(2,2,2-trifluoroethoxy)phenyl]-5-methoxy-2-oxopyridin-1(2H)-yl}butanoyl]amino}thiophene-2-carboxylic acid (enantiomer 2)

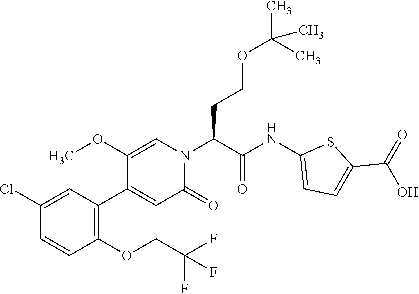 [SMILES: COc1cn([C@@H](CCOC(C)(C)C)C(=O)Nc2ccc(C(=O)O)s2)c(=O)cc1-c1cc(Cl)ccc1OCC(F)(F)F]

Enantiomer separation of 105 mg of 5-{[4-tert-butoxy-2-{4-[5-chloro-2-(2,2,2-trifluoroethoxy)-phenyl]-5-methoxy-2-oxopyridin-1(2H)-yl}butanoyl]amino}thiophene-2-carboxylic acid (racemate) gave 45.6 mg of enantiomer 1 (chiral HPLC: Rₜ=1.9 min) and 43 mg of the Example 69 title compound (enantiomer 2): chiral HPLC: Rₜ=4.1 min; 100% ee, 100% purity.

Separation method: column: Chiralpak AD-H 5 μm, 250 mm×20 mm; eluent: 75% carbon dioxide/25% ethanol; temperature: 40° C.; flow rate: 80 ml/min; pressure: 100 bar; UV detection: 210 nm.

Analysis: column: Daicel AD 5 μm, 250 mm×4.6 mm; eluent: 80% carbon dioxide, 20% ethanol; flow rate: 3 ml/min; UV detection: 210 nm.

LC-MS (Method 10): Rₜ=1.93 min; MS (ESIpos): m/z=617 [M+H]⁺

### Example 70

### 4-{[4-tert-Butoxy-2-{4-[5-chloro-2-(2,2,2-trifluoroethoxy)phenyl]-5-methoxy-2-oxopyridin-1(2H)-yl}butanoyl]amino}-2-methoxybenzoic acid (enantiomer 2)

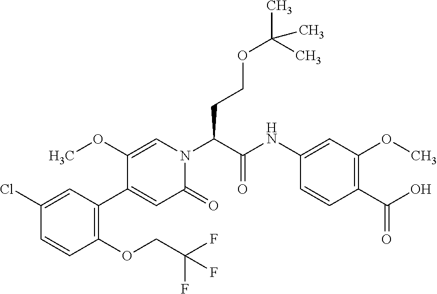 [SMILES: COc1cc(NC(=O)[C@H](CCOC(C)(C)C)n2cc(OC)c(-c3cc(Cl)ccc3OCC(F)(F)F)cc2=O)ccc1C(=O)O]

Enantiomer separation of 84 mg of 4-{[4-tert-butoxy-2-{4-[5-chloro-2-(2,2,2-trifluoroethoxy)-phenyl]-5-methoxy-2-oxopyridin-1(2H)-yl}butanoyl]amino}-2-methoxybenzoic acid (racemate) gave 32 mg of enantiomer 1 (chiral HPLC: Rₜ=2.8 min) and 27 mg of the Example 70 title compound (enantiomer 2): chiral HPLC: Rₜ=14.9 min; 100% ee, 100% purity.

Separation method: column: Chiralpak AZ-H 5 μm, 250 mm×20 mm; eluent: 75% carbon dioxide/25% ethanol; temperature: 40° C.; flow rate: 80 ml/min; pressure: 100 bar; UV detection: 210 nm.

Analysis: column: Daicel AZ 5 μm, 250 mm×4.6 mm; eluent: 70% carbon dioxide, 30% ethanol; flow rate: 3 ml/min; UV detection: 210 nm.

LC-MS (Method 1): Rₜ=1.09 min; MS (ESIpos): m/z=641 [M+H]⁺

### Example 71

### 6-{[4-tert-Butoxy-2-{4-[5-chloro-2-(2,2,2-trifluoroethoxy)phenyl]-5-methoxy-2-oxopyridin-1(2H)-yl}butanoyl]amino}imidazo[1,2-a]pyridine-2-carboxylic acid (enantiomer 2)

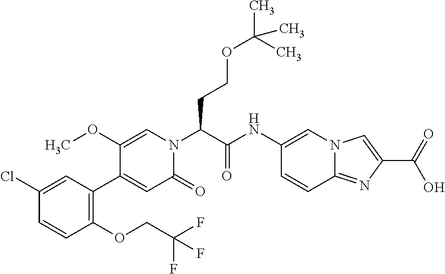 [SMILES: COc1cn([C@@H](CCOC(C)(C)C)C(=O)Nc2ccc3nc(C(=O)O)cn3c2)c(=O)cc1-c1cc(Cl)ccc1OCC(F)(F)F]

Enantiomer separation of 100 mg of 6-{[4-tert-butoxy-2-{4-[5-chloro-2-(2,2,2-trifluoroethoxy)-phenyl]-5-methoxy-2-oxopyridin-1(2H)-yl}butanoyl]amino}imidazo[1,2-a]pyridine-2-carboxylic acid (racemate) gave 36 mg of enantiomer 1 (chiral HPLC: Rₜ=8.0 min) and 33 mg of the Example 71 title compound (enantiomer 2): chiral HPLC: Rₜ=31.5 min; 100% ee, 100% purity.

Separation method: column: Chiralpak AZ-H 5 μm, 250 mm×20 mm; eluent: 75% carbon dioxide/25% ethanol; temperature: 40° C.; flow rate: 80 ml/min; pressure: 100 bar; UV detection: 210 nm.

Analysis: column: Daicel AZ 5 μm, 250 mm×4.6 mm; eluent: 80% carbon dioxide, 20% ethanol; flow rate: 3 ml/min; UV detection: 210 nm.

LC-MS (Method 1): Rₜ=0.93 min; MS (ESIpos): m/z=651 [M+H]⁺

### Example 72

### 5-({2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)thiophene-2-carboxylic acid (Diastereomer Mixture)

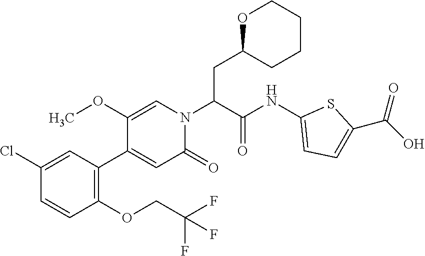 [SMILES: COc1cn(C(C[C@@H]2CCCCO2)C(=O)Nc2ccc(C(=O)O)s2)c(=O)cc1-c1cc(Cl)ccc1OCC(F)(F)F]

A solution of 55.0 mg (87.4 μmol) of methyl 5-({2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)thiophene-2-carboxylate (diastereomer mixture) in 4 ml of THF/water (3:1 mixture) was stirred in the presence of 7.34 mg (175 μmol) of lithium hydroxide monohydrate at room temperature for 18 hours and at 80° C. for 3 days. The mixture was then adjusted to pH 7 using aqueous hydrochloric acid solution (1N) and the THF was removed under reduced pressure.

The aqueous residue was diluted with acetonitrile and purified by preparative RP-HPLC (Reprosil C18, 0.1% strength formic acid/acetonitrile gradient). Yield: 32.3 mg (59% of theory)

LC-MS (Method 10): Rₜ=1.89 min; MS (ESIpos): m/z=615 [M+H]⁺

¹H-NMR (400 MHz, DMSO-d₆): δ [ppm]=12.65 (br s, 2H), 12.00 (s, 1H), 11.82 (s, 1H), 7.53-7.47 (m, 4H), 7.36-7.32 (m, 3H), 7.31 (s, 1H), 7.26 (dd, 2H), 6.82 (d, 1H), 6.79 (d, 1H), 6.33 (s, 1H), 6.31 (s, 1H), 5.79-5.73 (m, 1H), 5.66-5.57 (m, 1H), 4.80-4.71 (m, 4H), 3.89-3.80 (m, 2H), 3.60 (d, 6H), 3.28-3.11 (m, 4H), 2.96 (br t, 1H), 2.46-2.37 (m, 1H), 2.35-2.26 (m, 1H), 2.20-2.11 (m, 2H), 1.79-1.70 (m, 2H), 1.68-1.59 (m, 1H), 1.57-1.49 (m, 1H), 1.46-1.31 (m, 6H), 1.30-1.19 (m, 2H).

### Example 73

### 6-{[4-tert-Butoxy-2-{4-[5-chloro-2-(2,2,2-trifluoroethoxy)phenyl]-5-methoxy-2-oxopyridin-1(2H)-yl}butanoyl]amino}imidazo[1,2-a]pyridine-2-carboxylic acid (Racemate)

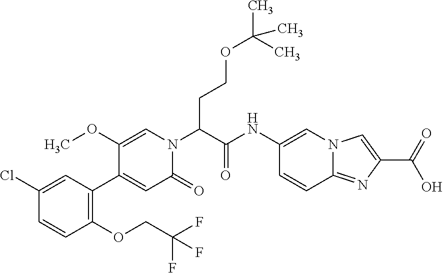 [SMILES: COc1cn(C(CCOC(C)(C)C)C(=O)Nc2ccc3nc(C(=O)O)cn3c2)c(=O)cc1-c1cc(Cl)ccc1OCC(F)(F)F]

To a solution of 116 mg (171 μmol) of ethyl 6-{[4-tert-butoxy-2-{4-[5-chloro-2-(2,2,2-trifluoroethoxy)phenyl]-5-methoxy-2-oxopyridin-1(2H)-yl}butanoyl]amino}imidazo [1,2-a]pyridine-2-carboxylate (racemate) in 3.7 ml of THF was added 1.0 ml of a lithium hydroxide monohydrate solution (0.50 M, 510 μmol), and the mixture was stirred at room temperature for 5 hours. The mixture was then adjusted to pH 7 using aqueous hydrochloric acid solution (1N) and the THF was removed under reduced pressure. The aqueous residue was diluted with acetonitrile and purified by preparative RP-HPLC (Reprosil C18, 0.1% strength formic acid/acetonitrile gradient). Yield: 100 mg (90% of theory).

LC-MS (Method 1): Rₜ=0.94 min; MS (ESIpos): m/z=651 [M+H]⁺

¹H-NMR (400 MHz, DMSO-d₆): δ [ppm]=10.63 (s, 1H), 9.31-9.28 (m, 1H), 8.53 (s, 1H), 7.60 (d, 1H), 7.49 (dd, 1H), 7.37 (dd, 1H), 7.33 (s, 1H), 7.30 (d, 1H), 7.26 (d, 1H), 6.33 (s, 1H), 5.82-5.75 (m, 1H), 4.82-4.67 (m, 2H), 3.60 (s, 3H), 2.40-2.34 (m, 2H), 1.04 (s, 9H).

### Example 74

### 4-{[4-tert-Butoxy-2-{4-[5-chloro-2-(2,2,2-trifluoroethoxy)phenyl]-5-methoxy-2-oxopyridin-1(2H)-yl}butanoyl]amino}-2-methoxybenzoic acid (Racemate)

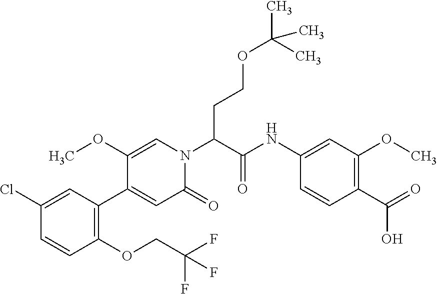 [SMILES: COc1cc(NC(=O)C(CCOC(C)(C)C)n2cc(OC)c(-c3cc(Cl)ccc3OCC(F)(F)F)cc2=O)ccc1C(=O)O]

95.0 mg (193 μmol) of 4-tert-butoxy-2-{4-[5-chloro-2-(2,2,2-trifluoroethoxy)phenyl]-5-methoxy-2-oxopyridin-1(2H)-yl}butanoic acid (racemate) and 34.2 mg (203 μmol) of 4-amino-2-methoxybenzoic acid in 1.1 ml of pyridine were reacted according to General Method 5. Yield: 86 mg (69% of theory).

LC-MS (Method 1): Rₜ=1.11 min; MS (ESIpos): m/z=641 [M+H]⁺

¹H-NMR (400 MHz, DMSO-d₆): δ [ppm]=12.31 (s, 1H), 10.64 (s, 1H), 7.68 (d, 1H), 7.56 (d, 1H), 7.49 (dd, 1H), 7.32 (s, 1H), 7.30 (d, 1H), 7.28-7.24 (m, 2H), 6.32 (s, 1H), 5.75 (dd, 1H), 4.83-4.67 (m, 2H), 3.78 (s, 3H), 3.60 (s, 3H), 3.41-3.34 (m, 1H), 3.28-3.20 (m, 1H), 2.44-2.29 (m, 2H), 1.03 (s, 9H).

### Example 75

### 5-{[4-tert-Butoxy-2-{4-[5-chloro-2-(2,2,2-trifluoroethoxy)phenyl]-5-methoxy-2-oxopyridin-1(2H)-yl}butanoyl]amino}thiophene-2-carboxylic acid (Racemate)

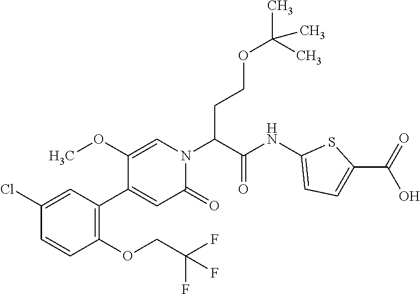 [SMILES: COc1cn(C(CCOC(C)(C)C)C(=O)Nc2ccc(C(=O)O)s2)c(=O)cc1-c1cc(Cl)ccc1OCC(F)(F)F]

A solution of 137 mg (217 μmol) of methyl 5-{[4-tert-butoxy-2-{4-[5-chloro-2-(2,2,2-trifluoroethoxy)phenyl]-5-methoxy-2-oxopyridin-1(2H)-yl}butanoyl]amino}thiophene-2-carboxylate (racemate) in 4 ml of THF/water (3:1 mixture) was stirred in the presence of 18.2 mg (434 μmol) of lithium hydroxide monohydrate at 80° C. for 18 hours. The mixture was then adjusted to pH 7 using aqueous hydrochloric acid solution (1N) and the THF was removed under reduced pressure. The aqueous residue was diluted with acetonitrile and purified by preparative RP-HPLC (Reprosil C18, 0.1% strength formic acid/acetonitrile gradient). Yield: 108 mg (80% of theory)

LC-MS (Method 10): Rₜ=1.96 min; MS (ESIpos): m/z=617 [M+H]⁺

¹H-NMR (400 MHz, DMSO-d₆): δ [ppm]=12.64 (br s, 1H), 11.98 (s, 1H), 7.53 (d, 1H), 7.49 (dd, 1H), 7.34-7.30 (m, 2H), 7.26 (d, 1H), 6.81 (d, 1H), 6.33 (s, 1H), 5.73 (dd, 1H), 4.84-4.67 (m, 2H), 3.61 (s, 3H), 3.41-3.34 (m, 1H), 3.26-3.18 (m, 1H), 2.44-2.28 (m, 2H), 1.03 (s, 9H).

### Example 76

### 4-({2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)-2-methoxybenzoic acid (Diastereomer Mixture)

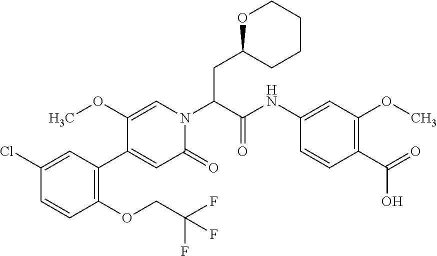 [SMILES: COc1cc(NC(=O)C(C[C@@H]2CCCCO2)n2cc(OC)c(-c3cc(Cl)ccc3OCC(F)(F)F)cc2=O)ccc1C(=O)O]

60 mg (85% purity, 104 μmol) of 2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoic acid (diastereomer mixture) and 17.6 mg (104 μmol) of 4-amino-2-methoxybenzoic acid in 580 μl of pyridine were reacted according to General Method 5. Yield: 35 mg (53% of theory).

LC-MS (Method 1): Rₜ=1.07 min; MS (ESIpos): m/z=639 [M+H]⁺

¹H-NMR (400 MHz, DMSO-d₆): δ [ppm]=12.32 (br s, 1H), 10.57 (2×s, 1H), 7.69-7.65 (m, 1H), 7.54-7.53 (m, 1H), 7.52-7.47 (m, 1H), 7.36-7.23 (m, 4H), 6.33-6.30 (m, 1H), 5.83-5.65 (m, 1H), 4.81-4.70 (m, 2H), 3.90-3.80 (m, 1H), 3.80-3.76 (m, 3H), 3.60 (s, 3H), 3.21-3.10 (m, 1H), 3.07-2.98 (m, 1H), 2.43-2.32 (m, 1H), 2.29-2.13 (m, 1H), 1.79-1.71 (m, 1H), 1.66-1.50 (m, 1H), 1.47-1.18 (m, 4H).

### Example 77

### 6-(2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)imidazo[1,2-a]pyridine-2-carboxylic acid (Diastereomer Mixture)

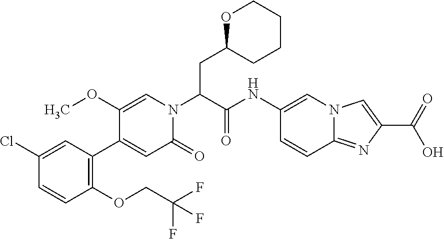 [SMILES: COc1cn(C(C[C@@H]2CCCCO2)C(=O)Nc2ccc3nc(C(=O)O)cn3c2)c(=O)cc1-c1cc(Cl)ccc1OCC(F)(F)F]

To a solution of 60 mg (88.6 μmol) of ethyl 6-({2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)imidazo[1,2-a]pyridine-2-carboxylate (diastereomer mixture) in 1.9 ml of THF were added 530 μl (0.50 M, 270 μmol) of aqueous lithium hydroxide solution, and the mixture was stirred at RT for 6 hours. Subsequently, the mixture was adjusted to pH 7 with aqueous hydrochloric acid solution (1 N). The solution was diluted with acetonitrile and purified by preparative RP-HPLC (Reprosil C18, 0.1% formic acid/acetonitrile gradient). Yield: 50 mg (87% of theory)

LC-MS (Method 1): Rₜ=0.89 min; MS (ESIpos): m/z=649 [M+H]⁺

¹H-NMR (400 MHz, DMSO-d₆): δ [ppm]=10.66 and 10.52 (2×s, 1H), 9.30-9.24 (m, 1H), 8.55-8.50 (m, 1H), 7.62-7.57 (m, 1H), 7.52-7.47 (m, 1H), 7.39-7.31 (m, 2H), 7.29-7.23 (m, 1H), 6.34-6.30 (m, 1H), 5.86-5.69 (m, 1H), 4.80-4.71 (m, 2H), 3.92-3.80 (m, 1H), 3.60 (s, 3H), 3.20-2.99 (m, 2H), 2.41-2.10 (m, 2H), 1.80-1.71 (m, 1H), 1.67-1.53 (m 1H), 1.47-1.20 (m, 4H).

### Example 78

### 4-({2-{4-[5-Chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)benzoic acid (diastereomer 2)

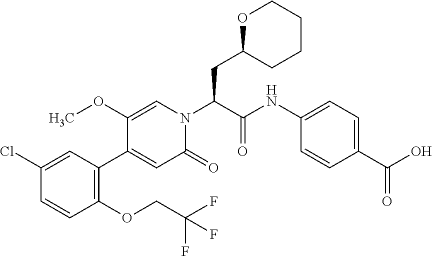 [SMILES: COc1cn([C@@H](C[C@@H]2CCCCO2)C(=O)Nc2ccc(C(=O)O)cc2)c(=O)cc1-c1cc(Cl)ccc1OCC(F)(F)F]

Diastereomer separation of 108 mg of 4-({2-{4-[5-chloro-2-(2,2,2-trifluoroethoxy)phenyl]-5-methoxy-2-oxopyridin-1(2H)-yl}-3-[(2S)-tetrahydro-2H-pyran-2-yl]propanoyl}amino)benzoic acid (diastereomer mixture) gave 51.8 mg of diastereomer 1 (chiral HPLC: Rₜ=4.0 min) and 44 mg of the Example 78 title compound (diastereomer 2): chiral HPLC: Rₜ=7.9 min; 100% ee, 100% purity.

Separation method: column: Chiralpak AZ-H 5 μm, 250 mm×20 mm; eluent: 75% carbon dioxide/25% ethanol; temperature: 40° C.; flow rate: 80 ml/min; pressure: 90 bar; UV detection: 210 nm.

Analysis: column: Daicel AZ 5 μm, 250 mm×4.6 mm; eluent: 70% carbon dioxide, 30% ethanol; flow rate: 3 ml/min; UV detection: 210 nm.

LC-MS (Method 1): Rₜ=1.04 min; MS (ESIpos): m/z=608 [M+H]⁺

¹H-NMR (400 MHz, DMSO-d₆): δ [ppm]=12.75 (br s, 1H), 10.71 (s, 1H), 7.90 (d, 2H), 7.76 (d, 2H), 7.49 (dd, 1H), 7.36 (s, 1H), 7.33 (d, 1H), 7.26 (d, 1H), 6.33 (s, 1H), 5.80 (t, 1H), 4.76 (q, 2H), 3.90-3.82 (m, 1H), 3.60 (s, 3H), 3.28-3.15 (m, 2H), 2.29-2.12 (m, 2H), 1.75 (br d, 1H), 1.63 (br d, 1H), 1.48-1.19 (m, 5H).

[symbol]

### B) ASSESSMENT OF PHYSIOLOGICAL EFFICACY

The suitability of the compounds according to the invention for treating thromboembolic disorders can be demonstrated in the following assay systems:

### a) Test Descriptions (In Vitro)

### a. 1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are conducted in microtitre plates.

Test substances are dissolved in dimethyl sulfoxide and serially diluted in dimethyl sulfoxide (3000 μM to 0.0078 μM; resulting final concentrations in the test: 50 μM to 0.00013 μM). 1 μl of the diluted substance solutions is placed into each of the wells of white microtitre plates from Greiner (384 wells). 20 μl of assay buffer (50 mM of Tris/HCl pH 7.4; 100 mM of sodium chloride; 5 mM of calcium chloride; 0.1% of bovine serum albumin) and 20 μl of factor XIa from Kordia (0.45 nM in assay buffer) are then added successively. After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nM, emission: 460 nM). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are given in Table A below (in some cases as averages from multiple individual determinations):

TABLE A

| Example No. | IC₅₀ [nM] | Example No. | IC₅₀ [nM] |
|---|---|---|---|
| 1 | 1.2 | 2 | 0.28 |
| 3 | 14 | 4 | 6.5 |
| 5 | 4.1 | 6 | 19 |
| 7 | 0.71 | 8 | 0.94 |
| 9 | 14 | 10 | 15 |
| 11 | 18 | 12 | 12 |
| 13 | 1.3 | 14 | 9.3 |
| 15 | 6.4 | 16 | 10 |
| 17 | 0.82 | 18 | 3.2 |
| 19 | 3.4 | 20 | 2.5 |
| 21 | 16 | 22 | 18 |
| 23 | 34 | 24 | 36 |
| 25 | 2.4 | 26 | 25 |
| 27 | 18 | 28 | 28 |
| 29 | 1.6 | 30 | 4.7 |
| 31 | 7.1 | 32 | 9.7 |
| 33 | 5.8 | 34 | 1.9 |
| 35 | 2.4 | 36 | 6.2 |
| 37 | 7.9 | 38 | 13 |
| 39 | 16 | 40 | 19 |
| 41 | 25 | 42 | 29 |
| 43 | 0.96 | 44 | 3.0 |
| 45 | 14 | 46 | 8.9 |
| 47 | 29 | 48 | 15 |
| 49 | 0.9 | 50 | 0.41 |
| 51 | 6.0 | 52 | 5.1 |
| 53 | 9.3 | 54 | 3.0 |
| 55 | 1.8 | 56 | 7.1 |
| 57 | 14 | 58 | 14 |
| 59 | 14 | 60 | 17 |
| 61 | 43 | 62 | 0.52 |
| 63 | 1.3 | 64 | 36 |
| 65 | 40 | 66 | 36 |
| 67 | 2.8 | 68 | 37 |
| 69 | 0.88 | 70 | 1.30 |
| 71 | 0.93 | 72 | 1.60 |
| 73 | 2.00 | 74 | 6.90 |
| 75 | 2.50 | 76 | 2.20 |
| 77 | 2.20 | 78 | 0.41 |

### a.2) Determination of the Selectivity

To demonstrate the selectivity of the substances with respect to FXIa inhibition, the test substances are examined for their inhibition of other human serine proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of NaCl, 0.1% BSA [bovine serum albumin], 5 mmol/l of calcium chloride, pH 7.4) and incubated for 15 min with test substance in various concentrations in dimethyl sulfoxide and also with dimethyl sulfoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulfoxide instead of test substance in dimethyl sulfoxide) and IC₅₀ values are calculated from the concentration/activity relationships.

### a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are conducted in the presence of varying concentrations of test substance or the corresponding solvent. To start the reaction, reagents from Thrombinoscope (30 μM or 0.1 μM recombinant tissue factor, 24 μM phospholipids in HEPES) are used. In addition, a thrombin calibrator from Thrombinoscope is used whose amidolytic activity is required for calculating the thrombin activity in a sample containing an unknown amount of thrombin. The test is conducted according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM Hepes, 60 mg/ml of BSA, 102 mM of calcium chloride, the thrombin generation is measured every 20 s over a period of 120 min. Measurement is conducted using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human plasma and rat plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboplastin time (APTT) is determined in the presence of varying concentrations of test substance or the corresponding solvent using a commercial test kit (PTT reagent from Roche). The test compounds are incubated with the plasma and the PTT reagent (cephalin, kaolin) at 37° C. for 3 minutes. Coagulation is then started by addition of 25 mM calcium chloride, and the time when coagulation occurs is determined. The concentration of test substance which effects an extension by 50% or a doubling of the APTT is determined.

### a.5) Determination of the Plasma Kallikrein Activity

To determine the plasma kallikrein inhibition of the substances according to the invention, a biochemical test system is used which utilizes the reaction of a peptidic plasma kallikrein substrate to determine the enzymatic activity of human plasma kallikrein. Here, plasma kallikrein cleaves from the peptic plasma kallikrein substrate the C-terminal aminomethylcoumarin (AMC), the fluorescence of which is measured. The determinations are conducted in microtitre plates.

Test substances are dissolved in dimethyl sulfoxide and serially diluted in dimethyl sulfoxide (3000 μM to 0.0078 μM; resulting final concentrations in the test: 50 μM to 0.00013 μM). 1 μl of the diluted substance solutions is placed into each of the wells of white microtitre plates from Greiner (384 wells). 20 μl of assay buffer (50 mM Tris/HCl pH 7.4; 100 mM sodium chloride solution; 5 mM calcium chloride solution; 0.1% of bovine serum albumin) and 20 μl of plasma kallikrein from Kordia (0.6 nM in assay buffer) are then added successively. After 15 min of incubation, the enzyme reaction is started by addition of 20 μl of the substrate H-Pro-Phe-Arg-AMC dissolved in assay buffer (10 μM in assay buffer) from Bachem, the mixture is incubated at room temperature (22° C.) for 30 min and fluorescence is then measured (excitation: 360 nM, emission: 460 nM). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are given in Table B below (in some cases as averages from multiple individual determinations):

TABLE B

| Example No. | IC₅₀ [nM] | Example No. | IC₅₀ [nM] |
|---|---|---|---|
| 1 | 55 | 2 | 15 |
| 3 | 100 | 4 | 49 |
| 5 | 33 | 6 | 130 |
| 7 | 34 | 8 | 19 |
| 9 | 57 | 10 | 77 |
| 11 | 87 | 12 | 60 |
| 13 | 14 | 14 | 45 |
| 15 | 35 | 16 | 67 |
| 17 | 47 | 18 | 55 |
| 19 | 38 | 20 | 120 |
| 21 | 150 | 22 | 170 |
| 23 | 340 | 24 | 130 |
| 25 | 120 | 26 | 150 |
| 27 | 95 | 28 | 120 |
| 29 | 80 | 30 | 74 |
| 31 | 120 | 32 | 160 |
| 33 | 66 | 34 | 45 |
| 35 | 130 | 36 | 70 |
| 37 | 72 | 38 | 93 |
| 39 | 56 | 40 | 230 |
| 41 | 130 | 42 | 190 |
| 43 | 9.4 | 44 | 320 |
| 45 | 280 | 46 | 150 |
| 47 | 310 | 48 | 150 |
| 49 | 33 | 50 | 19 |
| 51 | 65 | 52 | 82 |
| 53 | 210 | 54 | 90 |
| 55 | 32 | 56 | 120 |
| 57 | 54 | 58 | 57 |
| 59 | 110 | 60 | 84 |
| 61 | 150 | 62 | 15 |
| 63 | 14 | 64 | 59 |
| 65 | 140 | 66 | 180 |
| 67 | 140 | 68 | 250 |
| 69 | 8.90 | 70 | 58 |
| 71 | 10 | 72 | 24 |
| 73 | 18 | 74 | 210 |
| 75 | 19 | 76 | 150 |
| 77 | 23 | 78 | 19 |

### a.6) Determination of Endothelium Integrity

The activity of the compounds according to the invention is characterized by means of an in vitro permeability assay on “human umbilical venous cells” (HUVEC). Using the EOS apparatus (EC IS: Electric Cell-substrate Impedance Sensing; Applied Biophysics Inc; Troy, NY), it is possible to measure continuously variations in the transendothelial electrical resistance (TEER) across an endothelial cell monolayer plated over gold electrodes. HUVECs are shown on a 96-well sensor electrode plate (96W1 E, Ibidi GmbH, Martinsried, Germany). Hyperpermeability of the confluent cell monolayer formed is induced by stimulation with kininogen, prekallikrein and factor XII (100 nM each). The compounds according to the invention are added prior to the addition of the substances indicated above. The customary concentrations of the compounds are 1×10⁻¹⁰ to 1×10⁻⁶M.

### a.7) Determination of the In Vitro Permeability of Endothelial Cells

In a further hyperpermeability model, the activity of the substances on the modulation of macromolecular permeability is determined. HUVECs are shown on a fibronectin-coated Transwell filter membrane (24-well plates, 6.5 mm insert with 0.4 μM polycarbonate membrane; Costar #3413). The filter membrane separates the upper from the lower cell culture space, with the confluent endothelial cell layer on the floor of the upper cell culture space. 250 g/ml of 40 kDa FITC dextan (Invitrogen, D1844) are added to the medium of the upper chamber. Hyperpermeability of the monolayer is induced by stimulation with kininogen, prekallikrein and factor XII (100 nM each). Every 30 min, medium samples are removed from the lower chamber and relative fluorescence as a parameter for changes in macromolecular permeability as a function of time is determined using a fluorimeter. The compounds according to the invention are added prior to the addition of the substances indicated above. The customary concentrations of the compounds are 1×10⁻to 1×10⁶ M.

### b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the vessel injury is then caused by wrapping a piece of filter paper (10 mm×10 mm) on a Parafilm® strip (25 mm×12 mm) around the carotid artery without disturbing the blood flow. The filter paper contains 100 μl of a 13% solution of iron(II) chloride (Sigma) in water. After 5 min, the filter paper is removed and the vessel is rinsed twice with aqueous 0.9% sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3 mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

c) Determination of the Effect on Extravasation/Oedema Formation and/or Neovascularization in the Eye (In Vivo)

### c. 1) Test of the Efficacy of Substances in the Laser-Induced Choroidal Neovascularization Model

This study serves to investigate the efficacy of a test substance on reduction of extravasation/oedema formation and/or choroidal neovascularization in the rat model of laser-induced choroidal neovascularization.

To this end, pigmented rats of the Brown-Norway strain not showing any signs of ophthalmic disorders are selected and randomized into treatment groups. On day 0, the animals are anaesthetized by intraperitoneal injection (15 mg/kg xylazine and 80 mg/kg ketamine). Following instillation of a drop of a 0.5% strength tropicamide solution to dilate the pupils, choroidal neovascularization is triggered on six defined locations around the optical nerve using a 532 nm argon laser photocoagulator (diameter 50-75 μm, intensity 150 mW, duration 100 ms). The test substance and the appropriate vehicle (e.g. PBS, isotonic saline) are administered either systemically by the oral or intraperitonal route, or topically to the eye by repeated administration as eye drops or intravitreal injection. The body weight of all the animals is determined before the start of the study, and then daily during the study.

On day 21, an angiography is conducted using a fluorescence fundus camera (e.g. Kowe, HRA). Under anaesthesia and after another pupil dilation, a 10% strength sodium fluorescein dye is injected subcutaneously (s.c.). 2-10 min later, pictures of the eye background are taken. The degree of extravasation/the oedema, represented by the leakage of fluorescein, is assessed by two to three blinded observers and classified into degrees of severity from 0 (no extravasation) to 3 (strong colouration exceeding the actual lesion).

The animals are sacrificed on day 23, after which the eyes are removed and fixated in 4% paraformaldehyde solution for one hour at room temperature. After one washing, the retina is carefully peeled off and the sclera-choroidea complex is stained using an FITC isolectin B4 antibody and then applied flat to a microscope slide. The preparations obtained in this manner are evaluated using a fluorescence microscope (Apotom, Zeiss) at an excitation wavelength of 488 nm. The area or volume of the choroidal neovascularization (in μm² and μm³, respectively) is calculated by morphometric analysis using Axiovision 4.6 software.

### c.2) Test of the Efficacy of Substances in the Oxygen-Induced Retinopathy Model

It has been shown that oxygen-induced retinopathy is a useful animal model for the study of pathological retinal angiogenesis. This model is based on the observation that hyperoxia during early postnatal development in the retina causes arrest or delay of the growth of normal retinal blood vessels. When, after a 7-day hyperoxia phase, the animals are returned to normoxic room air, this is equivalent to relative hypoxia since the retina is missing the normal vessels which are required to ensure adequate supply of the neural tissue under normoxic conditions. The ischaemic situation caused in this manner results in an abnormal neovascularization which has some similarities with pathophysiological neovascularization in eye disorders such as wet AMD. In addition, the neovascularization caused is highly reproducible, quantifiable and an important parameter for examining the disease mechanisms and possible treatments for various forms of retinal disorders.

The aim of this study is to examine the efficacy of daily systemically administered doses of the test compound on the growth of retinal vessels in the oxygen-induced retinopathy model. Neonates of C57Bl/6 mice and their mothers are exposed to hyperoxia (70% oxygen) on postnatal day 7 (PD7) for 5 days. From PD12, the mice are kept under normoxic conditions (room air, 21% oxygen) until PD17. From day 12 to day 17, the mice are treated daily with the test substance or the corresponding vehicle. On day 17, all mice are anaesthetized with isoflurane and then sacrificed by cervical fracture. The eyes are removed and fixated in 4% formalin. After washing in phosphate-buffered saline, the retina is excised, a flat preparation thereof is produced and this is stained with isolectin B4 antibody. Quantification of neovascularization is conducted using a Zeiss ApoTome.

### C) WORKING EXAMPLES FOR PHARMACEUTICAL COMPOSITIONS

The substances according to the invention can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% solution (m/m) of the PVP in water. After drying, the granules are mixed with the magnesium stearate for 5 min. This mixture is compressed using a conventional tableting press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound of the invention.

### Production:

The Rhodigel is suspended in ethanol, and the compound of Example 1 is added to the suspension. The water is added while stirring. The mixture is stirred for about 6 h until swelling of the Rhodigel is complete.

### Solution or Suspension for Topical Administration to the Eye (Eye Drops):

A sterile pharmaceutical preparation for topical administration to the eye can be prepared by reconstituting a lyophilizate of the inventive compound in sterile saline. Suitable preservatives for such a solution or suspension are, for example, benzalkonium chloride, thiomersal or phenylmercuric nitrate in a concentration range of from 0.001 to 1 percent by weight.

### Solution or Suspension for Topical Administration to the Eye (Eye Drops):

A sterile pharmaceutical preparation for topical administration to the eye can be prepared by reconstituting a lyophilizate of the inventive compound in sterile saline. Suitable preservatives for such a solution or suspension are, for example, benzalkonium chloride, thiomersal or phenylmercuric nitrate in a concentration range of from 0.001 to 1 percent by weight.

1. Compound of the formula 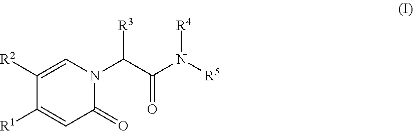
in which
R¹ is a group of the formula 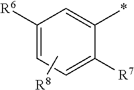
where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl, 3,3,3-trifluoro-2-methoxyprop-1-yl or 3,3,3-trifluoro-2-ethoxyprop-1-yl,
where alkyl may be substituted by a substituent selected from the group consisting of fluorine, hydroxyl, difluoromethyl, trifluoromethyl, methoxy, ethoxy, tert-butoxy, isopropoxy, difluoromethoxy, trifluoromethoxy, C₃-C₆-cycloalkyl, 4- to 6-membered oxoheterocyclyl, 1,4-dioxanyl, oxazolyl, pyrazolyl, 5,6-dihydro-4H-1,2-oxazin-3-yl, phenyl, pyridyl, C₃-C₆-cycloalkyloxy and 4- to 6-membered oxoheterocyclyloxy,
in which tert-butoxy and isopropoxy may be substituted by 1 to 3 fluorine substituents,
and
in which cycloalkyl may be substituted by 1 to 2 substituents selected independently from the group consisting of fluorine, hydroxyl, methyl, ethyl, methoxy, ethoxy, difluoromethyl, trifluoromethyl, difluoromethoxy and trifluoromethoxy,
and
in which oxoheterocyclyl may be substituted by 1 to 2 substituents selected independently from the group consisting of fluorine, methyl, ethyl, difluoromethyl and trifluoromethyl,
and
in which pyrazolyl is substituted by 1 or 2 substituents selected independently from the group consisting of fluorine, methyl and ethyl,
and
in which cycloalkyloxy and oxoheterocyclyloxy may be substituted by 1 to 2 substituents selected independently from the group consisting of fluorine and methyl,
R⁴ is hydrogen,
R⁵ is a group of the formula 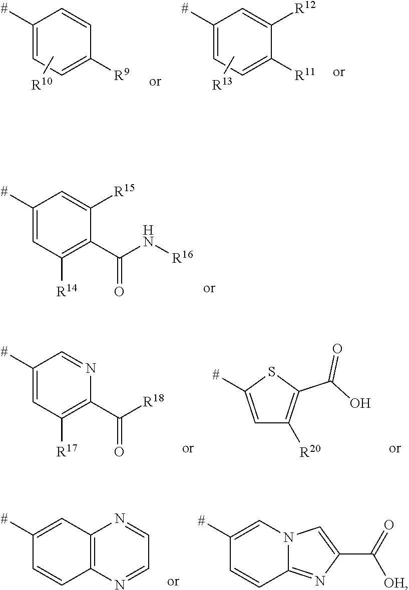
where # is the attachment point to the nitrogen atom,
R⁹ is hydroxycarbonyl or 5-membered heterocyclyl,
R¹⁰ is hydrogen, fluorine or methoxy,
R¹¹ and R¹² together with the carbon atoms to which they are bonded form a 5-membered heterocycle,
where the heterocycle may be substituted by 1 to 2 substituents selected independently from the group consisting of oxo, hydroxyl, hydroxycarbonyl, methyl, difluoromethyl and trifluoromethyl,
R¹³ is hydrogen or fluorine,
R¹⁴ is hydrogen or fluorine,
R¹⁵ is hydrogen or fluorine,
R¹⁶ is hydrogen, C₁-C₄-alkyl or cyclopropyl,
R¹⁷ is hydrogen or fluorine,
R¹⁸ is hydroxyl or —NHR¹⁹,
in which
R¹⁹ is hydrogen, C₁-C₄-alkyl or cyclopropyl,
R²⁰ is hydrogen or fluorine,
or one of the salts thereof, solvates thereof or solvates of the salts thereof.

2. Compound according to claim 1, wherein
R¹ is a group of the formula 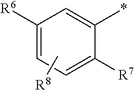
where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is C₁-C₅-alkyl,
where alkyl may be substituted by a substituent selected from the group consisting of methoxy, tert-butoxy, isopropoxy, trifluoromethoxy, 4- to 6-membered oxoheterocyclyl, 1,4-dioxanyl, pyrazolyl, 5,6-dihydro-4H-1,2-oxazin-3-yl and C₃-C₆-cycloalkyloxy,
in which pyrazolyl is substituted by a methyl substituent,
and
in which cycloalkyloxy may be substituted by 1 to 2 methyl substituents,
R⁴ is hydrogen,
R⁵ is a group of the formula 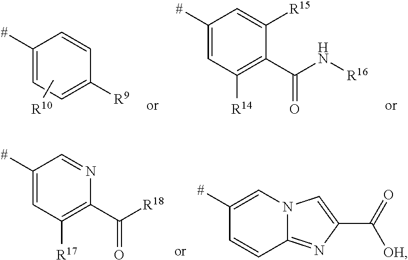
where # is the attachment point to the nitrogen atom,
R⁹ is hydroxycarbonyl,
R¹⁰ is hydrogen or fluorine,
R¹⁴ is hydrogen or fluorine,
R¹⁵ is hydrogen,
R¹⁶ is hydrogen, methyl or ethyl,
R¹⁷ is hydrogen or fluorine,
R¹⁸ is —NHR¹⁹,
in which
R¹⁹ is hydrogen, methyl or ethyl,
or
R⁵ is 2H-indazol-5-yl,
where the 5-membered heterocycle in 2H-indazol-5-yl may be substituted by a substituent selected from the group consisting of methyl, difluoromethyl and trifluoromethyl,
and
where the benzyl ring in 2H-indazol-5-yl may be substituted by a fluorine substituent,
or one of the salts thereof, solvates thereof or solvates of the salts thereof.

3. Compound according to either of claim 1, wherein
R¹ is a group of the formula 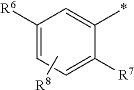
where * is the attachment point to the oxopyridine ring,
R⁶ is chlorine,
R⁷ is 2,2,2-trifluoroethoxy, 2,2-difluoroethoxy or 2-fluoroethoxy,
R⁸ is hydrogen,
R² is methoxy,
R³ is methyl,
where methyl is substituted by a substituent selected from the group consisting of tetrahydro-2H-pyranyl, 1,4-dioxanyl and pyrazolyl,
in which pyrazolyl is substituted by a methyl substituent,
or
is ethyl,
where ethyl is substituted by a substituent selected from the group consisting of methoxy,
or
is propyl,
where propyl is substituted by a methoxy substituent,
R⁴ is hydrogen,
R⁵ is a group of the formula 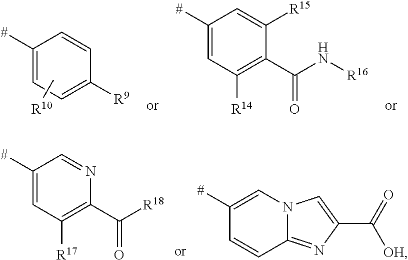
where # is the attachment point to the nitrogen atom,
R⁹ is hydroxycarbonyl,
R¹⁰ is hydrogen,
R¹⁴ is fluorine,
R¹⁵ is hydrogen,
R¹⁶ is hydrogen or methyl,
R¹⁷ is hydrogen,
R¹⁸ is —NHR¹⁹,
in which
R¹⁹ is hydrogen or methyl,
or
R⁵ is 2H-indazol-5-yl,
where the 5-membered heterocycle in 2H-indazol-5-yl is substituted by a methyl substituent,
or one of the salts thereof, solvates thereof or solvates of the salts thereof.

4. Process for preparing a compound of the formula (I) or one of the salts thereof, solvates thereof or solvates of the salts thereof according to claim 1, wherein either
[A] a compound of the formula 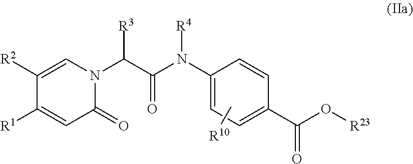
in which
R¹, R², R³, R⁴ and R¹⁰ have the definition given in claim 1, and
R²³ is tert-butyl
is reacted with an acid to give a compound of the formula 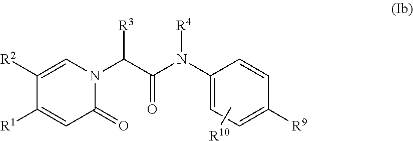
in which
R¹, R², R³, R⁴ and R¹⁰ have the definition given in claim 1, and
R⁹ is hydroxycarbonyl,
or
[B] a compound of the formula 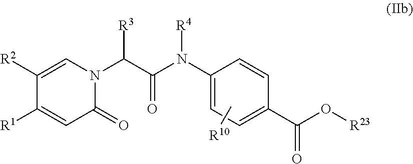
in which
R¹, R², R³, R⁴ and R¹⁰ have the definition given in claim 1, and
R²³ is methyl or ethyl,
is reacted with a base to give a compound of the formula 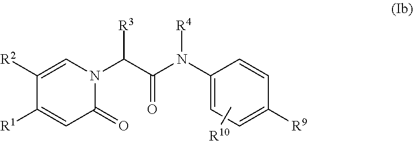
in which
R¹, R², R³, R⁴ and R¹⁰ have the definition given in claim 1, and
R⁹ is hydroxycarbonyl,
or
[C] a compound of the formula 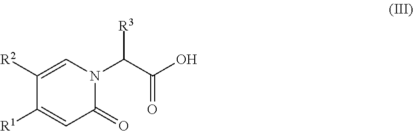
in which
R¹, R² and R³ have the definition given in claim 1
is reacted with a compound of the formula 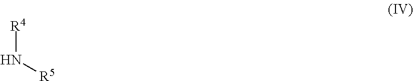
in which
R⁴ and R⁵ have the definition given in claim 1
in the presence of a dehydrating reagent to give a compound of the formula (I),
or
[D] a compound of the formula 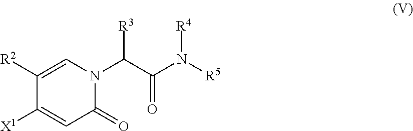
in which
R², R³, R⁴ and R⁵ have the definition given in claim 1, and
X¹ is chlorine, bromine or iodine
is reacted with a compound of the formula
R¹-Q¹ (VI)
in which
R¹ has the definition given in claim 1, and
Q¹ is —B(OH)₂, a boronic ester, preferably pinacol boronate, or —BF₃⁻K⁺,
under Suzuki coupling conditions to give a compound of the formula (I).

5. Compound according to claim 1, for treatment and/or prophylaxis of diseases.

6. Compound according to claim 1, for use in a method for treatment and/or prophylaxis of thrombotic or thromboembolic disorders, using a therapeutically active amount of the compound.

7. A method for producing a medicament for the treatment and/or prophylaxis of diseases, the method comprising producing the medicament with the compound of claim 1.

8. A method for production of a medicament for treatment and/or prophylaxis of thrombotic or thromboembolic disorders, of ophthalmic disorders, of hereditary angiooedema or inflammatory disorders of the intestine, such as Crohn's disease or ulcerative colitis, the method comprising producing the medicament with the compound of claim 1.

9. Medicament comprising a compound according to any of claim 1, in combination with an inert, non-toxic, pharmaceutically suitable auxiliary.

10. Medicament according to claim 9 for treatment and/or prophylaxis of thrombotic or thromboembolic disorders, of ophthalmic disorders, of hereditary angiooedema or inflammatory disorders of the intestine, such as Crohn's disease or ulcerative colitis.

11. Compound according to claim 2, for treatment and/or prophylaxis of diseases.

12. Compound according to claim 2, for use in a method for treatment and/or prophylaxis of thrombotic or thromboembolic disorders, using a therapeutically active amount of the compound.

13. A method for producing a medicament for the treatment and/or prophylaxis of diseases, the method comprising producing the medicament with the compound of claim 2.

14. Medicament comprising a compound according to claim 2, in combination with an inert, non-toxic, pharmaceutically suitable auxiliary.

15. Medicament according to claim 14 for treatment and/or prophylaxis of thrombotic or thromboembolic disorders, of ophthalmic disorders, of hereditary angiooedema or inflammatory disorders of the intestine, such as Crohn's disease or ulcerative colitis.

16. Compound according to claim 3, for treatment and/or prophylaxis of diseases.

17. Compound according to claim 3, for use in a method for treatment and/or prophylaxis of thrombotic or thromboembolic disorders, using a therapeutically active amount of the compound.

18. A method for producing a medicament for the treatment and/or prophylaxis of diseases, the method comprising producing the medicament with the compound of claim 3.

19. Medicament comprising a compound according to claim 3, in combination with an inert, non-toxic, pharmaceutically suitable auxiliary.

20. Medicament according to claim 19 for treatment and/or prophylaxis of thrombotic or thromboembolic disorders, of ophthalmic disorders, of hereditary angiooedema or inflammatory disorders of the intestine, such as Crohn's disease or ulcerative colitis.
